(19) United States
(12) Patent Application Publication
(10) Pub. No.: US 2017/0196954 A1
(43) Pub. Date: Jul. 13, 2017

(54) PRIME-BOOST REGIMENS WITH A TLR4 AGONIST ADJUVANT AND A LENTIVIRAL VECTOR

(71) Applicant: IMMUNE DESIGN CORP., Seattle, WA (US)

(72) Inventors: Richard KENNEY, Brisbane, CA (US); Frank HSU, Seattle, WA (US); Jan Henrik TER MEULEN, Mercer Island, WA (US); Peter Lars Aksel BERGLUND, Seattle, WA (US)

(21) Appl. No.: 15/326,052

(22) PCT Filed: Jul. 14, 2015
(86) PCT No.: PCT/US2015/040453

§ 371 Date: Jan. 13, 2017

Related U.S. Application Data
(60) Provisional application No. 62/024,797, filed on Jul. 15, 2014.
(60) Provisional application No. 62/024,792, filed on Jul. 15, 2014.

(51) Int. Cl. A61K 39/00; C12N 7/00; A61K 9/00

(52) U.S. Cl. CPC A61K 39/0011; A61K 9/0019; C12N 7/00; A61K 2039/5256; A61K 2039/5254; A61K 2039/545; A61K 2039/55566; A61K 2039/55572; C12N 2740/16043; C12N 2770/36122

(57) ABSTRACT

Compositions and methods are provided herein for improved dual immunization strategies that induce in a subject a robust immune response. The methods described are therefore useful for treating and/or preventing (i.e., reducing the likelihood or risk of occurrence) different diseases, disorders, and conditions such as cancers and infectious diseases for which induction of a humoral immune response and/or cellular immune response is desired and beneficial.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 48831_SeqListing.txt. The text file is 160,184 bytes, was created on 14 Jul. 2015, and is being submitted electronically via EFS-Web.

### BACKGROUND

Technical Field

The present disclosure relates generally to methods for enhancing the specific immune response to an immunogen by immunizing a subject with at least two compositions to induce humoral and cellular immune responses to the immunogen.

Description of the Related Art

The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite.

Cancer includes a broad range of diseases and affects approximately one in four individuals worldwide. A CTL response is a key feature of effective cancer vaccines; effective CD4 T cell help is also likely to play a critical role in productive CD8 T cell activation and thus provide clinical benefit. The autologous dendritic cell (DC)-based vaccine Sipuleucel-T (PROVENGE®) was recently approved by the U.S. Food and Drug Administration (FDA) for the treatment of metastatic, castrate-resistant prostate cancer though the survival benefit associated with this treatment is a modest 4.1 months, leaving significant need for improvement (see, e.g., Kantoff, et al., New Engl. J. Med. 363(5):411 (2010)). The poxvirus-vector based vaccine ProstVac® VF also shows a significant survival benefit in Phase II (see, e.g., Kantoff, et al., J. Clin. Oncol. 28(7):1099 (2010)). Active immune therapies such as Sipuleucel-T and ProstVac® VF have generally been better tolerated than the chemotherapeutic regimens that comprise the current standard of care for castrate-resistant disease (see, e.g., Petrylak, et al., N. Engl. J. Med. 351(15):1513 (2004); Sylwester, et al., J. Exp. Med. 202(5):673 (2005)). These clinical successes demonstrate that the immune response can be harnessed in a cancer setting to provide improved patient outcomes and extended survival.

With respect to microbial infections, malaria, tuberculosis, HIV-AIDS and other viral infections such as Herpes Simplex Virus (HSV) infections (the leading cause of genital ulcers worldwide) continue to contribute to global health concerns. HSV-2 prevalence is increasing at an alarming rate across the globe (see, e.g., Corey et al., J. Acquir. Immune Defic. Syndr. 35:435 (2004)). In the United States, the overall HSV-2 seroprevalence rate exceeds 20%, and in developing nations HSV-2 prevalence is estimated between 30% and 50%. In addition to the profound burden of HSV-2 infection in adults, the incidence of neonatal HSV-2 infection is increasing. Even when treated, neonatal encephalitis from HSV-2 infection has a mortality >15%, and the neurological morbidity among HSV-2 infected infants is an additional 30 to 50% of surviving cases. Concomitant with the HSV-2 epidemic is a stark realization that HSV-2 infection substantially increases the risk for HIV-1 acquisition and transmission. Data from Africa show that HSV-2 infection can increase the risk for HIV transmission by as much as 7-fold and that as many as half of newly acquired HIV cases are directly attributed to HSV-2 infection (see, e.g., Abu-Raddad et al., PLoS ONE 3(5):e2230 (2008)). Overall, the relative risk of HIV acquisition increases more than 2-fold in HSV-2-infected individuals.

The increasing prevalence of HSV-2 in the adult and pediatric populations persists despite the widespread use of pharmacological intervention. Antiviral medication given at high doses early in infection can reduce HSV transmission, but this does not prevent latent infection (see, e.g., Corey et al., Sex Transm. Dis. 12:215 (1985)). In a recent study, continuous suppressive administration with Valacyclovir reduced HSV transmission by less than 50% despite early intervention (see, e.g., Corey et al., N. Engl. J. Med. 350:11 (2004)). Alternatives to antiviral drugs, such as topical microbicides are unproven clinically. For these reasons, many leading authorities believe that vaccination is essential for diminishing the health impact of HSV-2 disease.

A need exists for vaccines, including improved vaccines, against infectious disease microorganisms, such as Human Immunodeficiency Virus (HIV) and Herpes Simplex Virus, malaria, antibiotic resistant bacteria, for which inducing a robust humoral and/or cell-mediated response is important for successful prevention and treatment of infection. In addition, despite positive impacts on survival of patients with cancer, a clear relationship between vaccine-induced tumor-specific immunity and patient benefit has not been conclusively demonstrated, indicating that considerable potential and need exists for improved cancer vaccine potency.

### BRIEF SUMMARY

One aspect of the present invention provides a method for inducing an immune response in a subject, the method comprising (a) administering to the subject at least two doses of a first immunogenic composition comprising (i) a lentiviral vector comprising a nucleotide sequence that encodes at least one immunogen or an immunogenic fragment thereof, wherein the at least one immunogen is capable of inducing an immune response specific for a first designated antigen; wherein the lentiviral vector is incorporated into a vector particle, and wherein the lentiviral vector particle is pseudotyped with an envelope glycoprotein that preferentially binds dendritic cells; (b) subsequently administering to the subject at least two doses of a second immunogenic composition comprising the at least one immunogen and a TLR4 agonist sequentially and at alternating times with two additional doses of the first immunogenic composition; thereby inducing an immune response specific for the first designated antigen. In one embodiment of the methods herein, the at least two doses of the first immunogenic composition are each administered as a single injection. In certain embodiments of the method the at least two doses are administered intradermally or subcutaneously. In another embodiment, the at least two doses of the first immunogenic composition are each administered as eight intradermal injections split between two injections over each deltoid and two injections over each quadricep. In a further embodiment, the at least two doses of the second immunogenic composition are administered following the at least two doses of the first immunogenic composition and are administered sequentially at different times from at least one additional dose of the first immunogenic composition. In one particular embodiment, the at least two doses of the first immunogenic composition are administered 2 to 3 weeks apart and wherein at least one dose of the second immunogenic composition is administered 2 to 3 weeks following the second dose of the first immunogenic composition. In another embodiment, subsequent doses of the first immunogenic composition and the second immunogenic composition are administered sequentially at different times. In certain embodiments of the methods herein, the at least two doses are administered intradermally or subcutaneously.

In another embodiment of the methods described herein, the at least two doses comprise from about 5×10⁸ to about 5×10¹⁰ vector genomes, or the at least two doses comprise from about 5×10⁹ to about 1×10¹⁰ vector genomes. In one embodiment, the at least a first dose comprises about 1×10¹⁰ vector genomes.

In one embodiment of the methods, the lentiviral vector is integration deficient. In other embodiments, the lentiviral vector is integration competent. In one particular embodiment of the methods described herein, the lentiviral vector is further characterized by any one or more or all of the following features: (a) pseudotyped with an envelope comprising a Sindbis virus E2 glycoprotein comprising an amino acid sequence having at least one amino acid change compared to SEQ ID NO:1, wherein residue 160 of SEQ ID NO:1 is either absent or an amino acid other than glutamic acid, and wherein the E2 glycoprotein is not a moiety of a fusion protein that comprises Sindbis virus E3 protein, (b) having a highly mannosylated envelope protein, optionally obtainable by culturing the packaging cells in a mannosidase inhibitor, (c) comprising a Vpx protein, optionally an SIVmac Vpx, (d) comprises a D64V integrase mutation within the gag/pol gene, (e) has a cPPT deletion within the vector genome, and (f) is optionally prepared using packaging cells comprising a rev-independent gag/pol system.

In certain embodiments of the methods described herein, the subject is human. In other embodiments, the subject is a mammal and may be a mammal of veterinary importance, such as cats, dogs, mice, horses, or cows. Non-human animals that may be treated using the methods described herein include non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In certain embodiments of the methods described herein, the subject has cancer and the first designated antigen is a tumor antigen. In this regard, the tumor antigen is selected from the group consisting of NY-ESO-1, MAGE-A3, MAGE-A1, MART-1/Melan-A, BAGE, RAGE, gp100, gp75, mda-7, tyrosinase, tyrosinase-related protein 2, renal cell carcinoma antigen, 5T4, SM22-alpha, carbonic anhydrase I, carbonic anhydrase IX, HIF-1alpha, HIF-2alpha, VEGF, prostate specific membrane antigen (PSMA), prostate-specific antigen (PSA), prostatic acid phosphates, six-transmembrane epithelial antigen of the prostate (STEAP), NKX3.1, telomerase enzyme, survivin, mesothelin, mutated ras, bcr/abl rearrangement, Her2/neu, mutated p53, wild-type p53, cytochrome P450 1B1, N-acetylglucosaminyltransferase-V, human papilloma virus protein E6, human papilloma virus protein E7, carcinoembryonic antigen, merkel cell virus T-antigen oncoproteins and alpha-fetoprotein. In one embodiment, the cancer is selected from the group consisting of renal cell carcinoma, prostate cancer, melanoma, and breast cancer.

In one embodiment of the methods described herein, the TLR4 agonist is a compound of the following structure:

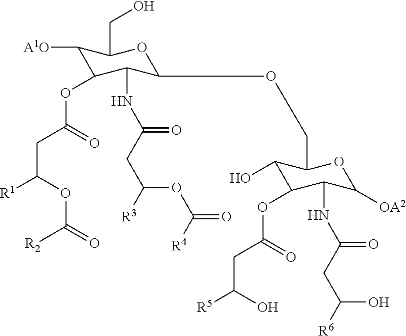

wherein A1 and A2 are independently selected from the group of hydrogen, phosphate, and phosphate salts and R1, R2, R3, R4, R5, and R6 are independently selected from the group of hydrocarbyl having 3 to 23 carbons, represented by C3-C23. In one specific embodiment, A1 is phosphate or phosphate salt, A2 is hydrogen, R1, R3, R5 and R6 are undecyl and R2 and R4 are tridecyl. In certain embodiments, the compound is formulated in a stable oil-in-water emulsion. In another embodiment, the second immunogenic composition of the methods herein comprises from about 5 μg GLA to about 10 μg GLA. In another embodiment, the second immunogenic composition of the methods herein comprises from about 5 μg GLA to about 10 μg GLA and each of the at least two doses of the first immunogenic composition comprises about 1×10¹⁰ vector genomes.

As used herein, the term “isolated” means that a material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector. A nucleic acid, which may be part of a vector, may still be isolated in that the nucleic acid is not part of the natural environment for the nucleic acid. An isolated polypeptide or protein, or fragment thereof, could be a component of a composition, and still be isolated in that the composition is not part of the natural environment for the polypeptide. The term “gene” means the segment of DNA involved in producing a polypeptide chain; a gene includes regions preceding and following the coding region “leader and trailer” as well as intervening sequences (introns) between individual coding segments (exons). Amino acids may be referred to herein according to the single letter and three letter codes, which are common textbook knowledge in the art, and therefore with which a person skilled in the art is familiar. The term “fusion polypeptide” used herein may also be used interchangeably with “fusion protein,” and unless specifically indicated otherwise, the two terms are not meant to indicate molecules that have distinguishable properties or characteristics.

In one embodiment of the present disclosure, a method of treating a mammal suffering from cancer is provided comprising administering to the mammal a first dose of a composition comprising a lentivector pseudotyped with an alphavirus envelope that preferentially binds dendritic cells, wherein the lentivector comprises an exogenous polynucleotide encoding a tumor antigen; a second dose of the composition comprising the lentivector of (a); and optionally, a third dose of the composition comprising the lentivector of (a); wherein the composition comprising the lentivector is administered by intradermal injection; wherein the first dose comprises 5×10⁸ to 5×10¹⁰ vector genomes; the second dose comprises 5×10⁸ to 5×10¹⁰ vector genomes, and the third dose comprises 5×10⁸ to 5×10¹⁰ vector genomes. In another embodiment, the mammal is human.

In another embodiment, the aforementioned method is provided wherein the first and second dose, and/or the second and third dose are separated by a time selected from the group consisting of 1, 2, 3, 4, 5 or 6 weeks.

In still another embodiment, the aforementioned is provided wherein the tumor antigen is selected from the group consisting of NY-ESO-1, MAGE-A3, MAGE-A1, MART-1/Melan-A, BAGE, RAGE, gp100, gp75, mda-7, tyrosinase, tyrosinase-related protein 2, renal cell carcinoma antigen, 5T4, SM22-alpha, carbonic anhydrase I, carbonic anhydrase IX, HIF-1alpha, HIF-2alpha, VEGF, prostate specific membrane antigen (PSMA), prostate-specific antigen (PSA), prostatic acid phosphates, six-transmembrane epithelial antigen of the prostate (STEAP), NKX3.1, telomerase enzyme, survivin, mesothelin, mutated ras, bcr/abl rearrangement, Her2/neu, mutated p53, wild-type p53, cytochrome P450 1B1, N-acetylglucosaminyltransferase-V, human papilloma virus protein E6, human papilloma virus protein E7, carcinoembryonic antigen, merkel cell virus T-antigen oncoproteins and alpha-fetoprotein.

In yet another embodiment, the aforementioned is provided wherein the first dose, the second dose and the third dose are divided into eight intradermal injections. In another embodiment, the first dose, the second dose and the third dose are administered at least 3 cm between each site of intradermal injection.

In another embodiment, the aforementioned is provided wherein the lentivector is integration deficient. In still another embodiment, the aforementioned is provided wherein the lentivector is integration competent.

In still another embodiment, an aforementioned is provided wherein the lentivector is further characterized by any one or more or all of the following features: (a) pseudotyped with an envelope of SEQ ID NO: 30, (b) having a highly mannosylated envelope protein, optionally obtainable by culturing the packaging cells in a mannosidase inhibitor, (c) comprising a Vpx protein, optionally SIVmac Vpx of SEQ ID NO: 44, (d) comprises a D64V integrase mutation within the gag/pol gene, (e) has a cPPT deletion within the vector genome, and (f) is optionally prepared using packaging cells comprising a rev-independent gag/pol system.

In yet another embodiment, an aforementioned is provided wherein the cancer is selected from the group consisting of renal cell carcinoma, prostate cancer, melanoma, and breast cancer. Additional cancers contemplated in the present disclosure include, but are not limited to, lung cancer, cervical cancer, ovarian cancer, uterine cancer, liver cancer, gastric cancer, colon cancer, pancreatic cancer, kidney cancer, bladder cancer, brain cancer, fibrosarcoma, myxosarcoma, liposarcoma, chondrosarcoma, osteogenic sarcoma, chordoma, angiosarcoma, endotheliosarcoma, lymphangiosarcoma, pseudomyxoma petitonei, lymphangioendotheliosarcoma, synovioma, mesothelioma, Ewing's tumor, leiomyosarcoma, rhabdomyosarcoma, colon carcinoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma and Wilms' tumor. In certain other related embodiments the cancer cell originates in a cancer that is selected from testicular tumor, lung carcinoma, small cell lung carcinoma, bladder carcinoma, epithelial carcinoma, glioma, glioblastoma multiforme, astrocytoma, plasmocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, Waldenstrom's macroglobulinemia or other cancers.

The present disclosure also contemplates the treatment of cancer using lentiviral vector particles that exhibit two characteristics (a) pseudotyped with a highly mannosylated alphavirus glycoprotein and (b) comprising a Vpx protein, have unexpectedly improved transduction efficiency for cells expressing DC-SIGN. These particles infect cells expressing DC-SIGN, particularly dendritic cells, significantly more efficiently than lentiviral vector particles having only one of these two characteristics. In particular instances, highly mannosylated pseudotyped lentiviral vector particles are provided that comprise a Vpx protein and a lentiviral genome comprising a sequence of interest (e.g., a polynucleotide encoding an antigen).

One aspect of the present disclosure provides a method of treating a cancer in a mammal comprising administering at least a first dose of a composition comprising a lentiviral vector pseudotyped with an envelope glycoprotein that preferentially binds dendritic cells, wherein the lentiviral vector comprises an exogenous polynucleotide encoding a tumor antigen and wherein the at least a first dose is administered in a single injection. In one embodiment of the method, the mammal is human. In another embodiment of the method, multiple doses of the lentiviral vector particles are administered, such as at least 2, 3, 4, 5, 6, or more. In some embodiments, each dose is administered from every week to every three weeks. In other embodiments, each dose is administered every week, every two weeks, every three weeks, every 4 weeks, every 5 weeks, every 6 weeks, every 7 weeks or every 8 weeks. In certain embodiments of the methods herein, the at least a first dose comprises from about 5×10⁸ to about 5×10¹⁰ vector genomes or in other embodiments wherein the at least a first dose comprises from about 5×10⁹ to about 1×10¹⁰ vector genomes. In one embodiment, the at least a first dose comprises about 1×10¹⁰ vector genomes.

In certain embodiments of the methods, the immune response specific for the tumor antigen elicited after administration of a first dose of the lentiviral vector as a single injection is greater than the immune response specific for the tumor antigen elicited after administration of the first dose split into multiple injections. In one embodiment, the administration of the first dose split into separate sites is separated into 2-4 sites. In particular embodiment the at least a first dose is administered intradermally or subcutaneously.

As used herein and in the appended claims, the singular forms “a,” “and,” and “the” include plural referents unless the context clearly dictates otherwise. Thus, for example, reference to “an antigen” includes a plurality of such antigens, and reference to “a cell” or “the cell” includes reference to one or more cells and equivalents thereof (e.g., plurality of cells) known to those skilled in the art, and so forth. Similarly, reference to “a compound” or “a composition” includes a plurality of such compounds or compositions, and refers to one or more compounds or compositions, respectively, unless the context clearly dictates otherwise. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### BRIEF DESCRIPTION OF THE DRAWINGS

FIG. 1. Neutralizing antibody responses against ID-VP02 can be detected after immunization. (a) Ten-fold dilutions of serum from mice immunized with ID-VP02 (ID-VP02-GFP), or from control mice injected with HBSS or VSV-G pseudotyped vector (VSV-G-GFP) at the indicated doses, were pre-incubated with a reporter vector (ID-VP02 encoding GFP) for 1 hour. This serum-vector mix was then used as test article in a GFP transduction assay utilizing 293T.huDC-SIGN target cells. The percentage of GFP positive cells was analyzed 2 days post-transduction. The results are presented as mean±SD of three mice per group. (b) Groups of mice were first immunized with either 7.5×10¹⁰, 3.0×10⁹, or 1.2×10⁸ vector genomes of ID-VP02 encoding GFP, 7.5×10¹⁰ vector genomes of VSV-G pseudotyped lentivector encoding GFP, or HBSS. On day 28-post primary immunization the animals were immunized with 3.0×10⁹ of ID-VP02 encoding an alternative antigen cassette, LV1b. OVA₂₅₇-specific CD8 T-cell response in the spleen was measured by intracellular cytokine staining (ICS) on day 12 post-second immunization.

FIG. 2. Schematic representation of the immunization regimen used in the pivotal safety-toxicity study of CMB305 in BALB-c mice. Arrows marked with (L) or (G) indicate dosing events with ID-LV305 (L) and IDC-G305 (G), respectively. Arrows marked with (N) indicate the terminal, intermediate recovery, and final recovery necropsies at day 57, 67 and 85.

FIG. 3. Immunogenicity of a 7-Dose CMB305 Study Regimen in BALB/c Mice in a Repeat-Dose Safety/Toxicity Study at 2 Time Points Post-Immunization. BALB/c mice (N=10 female and 10 male per treatment group and necropsy time) were immunized at the base of the tail s.c with 2.5×10⁸ genomes of LV305 on Days 1, 15, 29, and 43 and with i.m. G305 (5 μg NY-ESO-1 protein in 5 μg GLA-SE) on Days 22, 36, 50. At days 57 and 67, splenic NY-ESO-1-specific CD8 T-cell responses were measured by interferon gamma ELISpot (panel a) or intracellular cytokine staining for IFN-γ, TNF, and IL-2 after ex vivo restimulation with the H-2D^{d}-restricted NY-ESO-1 epitope RGPESRLL (NY-ESO-1₈₁₋₈₈) (panels b, c). Splenocytes from mice immunized with vehicle controls and restimulated with NY-ESO-1₈₁₋₈₈ served as negative controls. The median percentage of CD8 T cells producing IFN-γ (FIG. 3A, FIG. 3B) or each combination of cytokines, as indicated, (FIG. 3c) is depicted for each group. These results demonstrated that this dosing regimen elicited detectable levels of anti-NY-ESO-1 specific CTL responses at the time points sampled. Note that the groups of male mice dosed with CMB305 are composed of 9 animals. Vertical dashed bars indicate that the responses at Days 57 and 67 were assayed separately and, thus, that the figures represent data generated from separate experiments. In FIG. 3C: Group 1=Vehicle; Group 2=CMB305. Horizontal bars in panels a and b represent arithmetic means with error bars representing one standard deviation. Error bars in panel c represent SEM of each population.

FIG. 4. Groups of five female B6D2F1 mice were immunized with 1.25×, 2.5×, 5×, 10×, or 20×10⁸ vector genomes of ID-LV305, as indicated. The mice were immunized once in the upper back (bars 2 to 6, labeled ‘1U’), once in the lower back (bars 7-11, labeled ‘1L’), or four sites (bars 12-14, the three highest dosage levels only). At days 14 post-immunization, animals were sacrificed, and splenocytes analyzed for CD8-cell responses against NY-ESO-1 using re-stimulation with NY-ESO-1 derived peptides followed by ICS and flow cytometry. Bars represent the percentages of cytokine-producing CD8-positive T cells. Stimulation with an MHC I-binding control peptide resulted in response levels below 0.1% (not shown). At day 12 post immunization, the percentage of AH1A5- or AH1-specific splenic CD8 T cells was measured by ICS.

FIG. 5. Diagram showing a planned clinical regimen for CMB305 in which ID-LV305 (lentiviral vector expressing NY-ESO-1) and IDC-G305 (recombinant NY-ESO-1 protein in combination with GLA-SE) used in the CMB305 combination therapy will be administered sequentially at different times. In particular, LV305 is administered at day 0 and at day 21 followed by administration of G305 at day 35. Another dose of lentiviral vector is given at day 49 followed by another administration of G305 at day 63. A fourth dose of LV305 is given at day 77 followed by a third dose of G305 at day 91.

FIG. 6 illustrates several exemplary immunization regimens that may be employed using the immunogenic compositions described herein for inducing immune responses.

FIG. 7: BALB/c mice (n=10/group) were immunized with 4×10⁹ or 2×10¹⁰ vector genomes of ID-VP02-NY-ESO-1.21 days post immunization, immunized and untreated control mice were injected subcutaneously on the right flank with 7.5×10⁵ CT26-NY-ESO-1 tumor cells in Matrigel® or with Matrigel® alone as a negative control. Nine days after injection, the implants were excised and weighed to assess tumor growth.

FIG. 8. Groups of four female BALB/c mice were immunized twice two weeks apart with either VP02 encoding GFP at 5×10⁸ vector genomes or with ID-LV305 at 2-fold escalating dosage levels, between 1.25×10⁸ and 5×10⁸ vector genomes, as indicated. At 9 days post-boost, NY-ESO-1 splenic CD8 T cell responses were measured by surface staining of CD107a and intracellular cytokine staining (ICS) for IFN-γ, TNF, and IL-2 after ex vivo restimulation with the H-2D^{d}-restricted NY-ESO-1 epitope RGPESRLL (NY-ESO-1₈₁₋₈₈). Splenocytes from mice immunized with HBSS vehicle control and restimulated with NY-ESO-1₈₁₋₈₈ served as negative controls. The average percentage of CD8 T cells producing each combination of cytokines is depicted for each group.

### DETAILED DESCRIPTION

Vaccination against diseases and conditions, such as infectious diseases, has included strategies in which subjects are immunized with one composition (the priming composition) and subsequently immunized with a different composition (boosting composition). However, the dual vaccination strategies to date have not adequately induced both CD4 and CD8 T cell responses as well as humoral immunity that provide sufficient protection against many diseases and conditions.

Provided herein are compositions and methods for improved dual immunization strategies that induce in a subject an immune response that includes a humoral immune response and cellular immune response, both CD4 and CD8 T lymphocyte immune responses, providing a complete adaptive immune response to one or more antigens. Accordingly, the compositions described herein may be developed and formulated as vaccines. The methods described are therefore useful for treating and preventing (i.e., reducing the likelihood or risk of occurrence or recurrence in a biologically, clinically, and/or statistically significant manner) different diseases, disorders, and conditions such as cancers and infectious diseases for which induction of both a humoral immune response and cellular immune response improves the clinical outcome or is necessary for optimal benefit.

Provided herein are two immunogenic compositions that are administered concurrently or sequentially in either order to a subject in need thereof. At least one of the immunogenic compositions induces a specific humoral (i.e., antibody response) and/or a specific CD4 T cell response (which may include a memory CD4 T cell response) to an immunogen, and at least one of the immunogenic compositions induces a specific CD8 T cell response, which may include a cytotoxic T cell (CTL) response, specific for the immunogen. In certain embodiments, one of the two immunogenic compositions may be more effective for inducing a specific humoral and/or specific CD4 T cell response and the other of the two immunogenic compositions may be more effective for inducing a specific CD8 T cell response.

One immunogenic composition comprises at least one immunogen that is capable of inducing an immune response specific for a designated antigen of interest. This immunogenic composition may further comprise an adjuvant that enhances, or that may be required, for inducing an immune response specific for the immunogen and the designated antigen. A second immunogenic composition comprises a recombinant expression vector comprising a nucleotide sequence that encodes the immunogen. The recombinant expression vector further comprises at least one regulatory sequence operatively linked to the nucleotide sequence that encodes the immunogen and, thus, the recombinant expression vector is capable of directing expression of the immunogen. In certain specific embodiments, the recombinant expression vector is incorporated into a vector particle (e.g., a virus vector particle). As described further herein, the immunogen may be an immunogenic fragment of the designated antigen or may be the full-length designated antigen (or an immunogenic variant thereof), or a fusion protein that comprises one or more immunogenic fragments or that comprises the full-length designated antigen (or immunogenic variant thereof).

In other specific embodiments provided herein, a second immunogen is included in either immunogenic composition. The second immunogen is capable of inducing an immune response to a designated antigen, which may be the same or different from the designated antigen for which the first immunogen induces a specific immune response. In another specific embodiment, the first immunogen induces a specific CD4 T cell immune response and may also induce a specific antibody response, and the second immunogen induces at least a CD8 T cell immune response. In certain particular embodiments, the subject to be immunized is intended to be immunized with the second immunogen only via expression of the second immunogen by the recombinant expression vector. Accordingly, the immunogenic composition comprising the first immunogen (and which may further comprise an adjuvant) lacks the second immunogen, and the recombinant expression vector comprises a nucleotide sequence that encodes the first immunogen and that encodes a second immunogen.

The immunogenic compositions and methods described herein may be useful for preventing or treating an infectious disease, particularly infectious diseases for which no satisfactory vaccine or post-infection treatment is available (for example, viral infections such as HIV and HSV-2, and parasitic infections such as malaria). In other embodiments, the immunogenic compositions and methods described herein may be used for treating and/or reducing the likelihood of occurrence of a cancer and malignancy.

The various embodiments of the immunogenic compositions, preparations comprising the immunogenic compositions, and methods of using the preparations and compositions are described in detail below.

### Immunogenic Compositions

Different immunogenic compositions are described herein that when used in a coordinated immunization strategy are useful for inducing specific, adaptive immune responses. One immunogenic composition comprises at least one immunogen and may further comprise a physiologically suitable (i.e., pharmaceutically acceptable or suitable) adjuvant. The immunogen included in this first immunogenic composition is typically an isolated immunogen, which may be isolated from its natural environment or may be recombinantly produced. For ease of reference the immunogen present in the first immunogenic composition is called herein an isolated/recombinant immunogen. A second, different composition comprises a recombinant expression vector that comprises a nucleotide sequence encoding the immunogen. The second immunogenic composition may also further comprise an adjuvant. If both the first composition and second composition comprise an adjuvant, the adjuvant included in each composition may be the same or different.

Administration of the two different compositions to a subject induces a specific immune response to the at least one immunogen and to a respective antigen of interest (also called herein a designated antigen). The specific immune response includes a specific humoral immune response (i.e., specific antibody response) and a specific cellular immune response (including a CD4 T cell response and a CD8 T cell response), each response specific for the immunogen and thereby specific for the designated antigen of interest. An immunogenic preparation referred to herein, comprises these two immunogenic compositions, which may be referred to herein for convenience as a first immunogenic composition and a second immunogenic composition. Accordingly, in one embodiment, an immunogenic preparation comprises (a) at least one immunogenic composition that comprises at least one isolated/recombinant immunogen capable of eliciting an immune response specific for a designated antigen; and (b) at least one second immunogenic composition that comprises a recombinant expression vector comprising a nucleotide sequence encoding the at least one immunogen. The immunogenic compositions of the preparations may be administered concurrently or sequentially in either order to a subject to induce an immune response specific for the immunogen and for the respective designated antigen. Each of the immunogenic compositions and uses for the compositions are described in greater detail herein.

Each immunogenic composition may further comprise at least one physiologically (or pharmaceutically) acceptable or suitable excipient. Any physiologically or pharmaceutically suitable excipient or carrier (i.e., a non-toxic material that does not interfere with the activity of the active ingredient) known to those of ordinary skill in the art for use in pharmaceutical compositions may be employed in the immunogenic compositions described herein. Exemplary excipients include diluents and carriers that maintain stability and integrity of proteins. Excipients for therapeutic use are well known, and are described, for example, in Remington: The Science and Practice of Pharmacy (Gennaro, 21^{st} Ed. Mack Pub. Co., Easton, Pa. (2005)), and are described in greater detail herein.

The immunogen may be identical to the designated antigen, that is, the immunogen comprises an exemplary full-length amino acid sequence of the designated antigen, or may comprise a variant thereof that shares a high percent identity with the exemplary full-length designated antigen and that retains a functional characteristic of the designated antigen, for example, the capability to induce a specific immune response. Alternatively, an immunogen may be an immunogenic fragment of the designated antigen. Immunogens that are variants or fragments of a designated antigen exhibit the capability to induce an immune response (e.g., a humoral response (i.e., B cell response) or a cell-mediated response (i.e., T cell response (including a cytotoxic T lymphocyte response)) or both a humoral and cell-mediated response in a subject in a statistically, clinically, and/or biologically significant manner Designated antigens of interest and immunogenic fragments and immunogenic variants of designated antigens thereof are described in greater detail herein.

With respect to the immunogenic composition that comprises at least one isolated/recombinant immunogen, the immunogen may be a polypeptide or peptide that has been recombinantly produced in a host cell and then isolated from the host cell or isolated from the host cell culture (i.e., removed from its original host cell environment) according to methods routinely practiced in the molecular biology and protein isolation arts. When the immunogen is recombinantly produced according to methods described herein and in the art and with which a skilled person is familiar, the immunogen may be called a recombinant immunogen. Alternatively, the immunogen may be isolated or removed from a natural source, such as, for example, a virus, bacteria, parasite, fungus, or tumor cell. Methods for isolating one or more immunogens and antigens from natural sources are described in the art and also may be readily empirically determined by a skilled person using methods and techniques routinely practiced in the art.

As described herein, the recombinant expression vector included in the second immunogenic composition comprises a nucleotide sequence (also called herein a polynucleotide sequence) that encodes the at least one immunogen. The recombinant expression vector further comprises at least one regulatory sequence that is operatively linked to the encoding nucleotide sequence such that the vector is capable of directing expression of the immunogen. The immunogen that is encoded and expressed by the recombinant expression vector may be identical to the designated antigen, that is, the immunogen comprises an exemplary full-length amino acid sequence of the designated antigen, or may comprise a variant thereof that shares a high percent identity with the exemplary full-length designated antigen and that retains a functional characteristic of the designated antigen, for example, the capability to induce a specific immune response. Alternatively, the encoded immunogen may be an immunogenic fragment of the designated antigen Immunogens that are variants or fragments of a designated antigen exhibit the capability to induce an immune response (e.g., a humoral response (i.e., B cell response) or a cell-mediated response (i.e., T cell response (including a cytotoxic T lymphocyte response)) or both a humoral and cell-mediated response in a subject in a statistically, clinically, and/or biologically significant manner Designated antigens of interest and immunogenic fragments and immunogenic variants of designated antigens thereof are described in greater detail herein.

In certain embodiments, the recombinant expression vector is incorporated into a vector particle (e.g., a virus vector particle or a cell particle). The recombinant expression vector or vector particle comprising the vector is constructed in a manner that enables the particle to be introduced into (i.e., delivered to) a target cell. In certain embodiments, the target cell is an antigen-presenting cell. In more specific embodiments, the target cell is a professional antigen-presenting cell such as a dendritic cell. The immunogen is then expressed in the target cell, and the immunogen or a fragment thereof is presented on the surface of the antigen-presenting cell and induces an immune response specific for the immunogen and thereby for the respective designated antigen.

In other embodiments, the immunogenic composition that comprises at least one isolated/recombinant immunogen (and which may further comprise an adjuvant) further comprises at least one additional immunogen (i.e., at least two, three, four, five, or more immunogens which may be restated as two, three, four, five, or more immunogens)). In certain embodiments, an immunogenic composition may comprise two or more isolated/recombinant immunogens (i.e., at least two immunogens), forming a multivalent immunogenic composition. In instances when the two or more immunogens are combined with an adjuvant, the immunogenic composition may comprise each immunogen formulated separately with an adjuvant and then the adjuvanated immunogens are combined to form the immunogenic composition. Alternatively, the two or more immunogens may be formulated together with an adjuvant. In certain specific embodiments, each of the additional immunogens (e.g., the second, third, etc immunogen) may induce an immune response to the same designated antigen as the first immunogen. In other specific embodiments, each of the additional immunogens (e.g., the second, third, etc. immunogen) may induce an immune response specific for a different (e.g., a second, third, etc.) designated antigen, respectively.

In certain alternative embodiments, a multivalent immunogenic composition may comprise a cell lysate, cell organelle, or cell supernatant that includes at least two immunogens. For example, immunogens removed from their original environment, such as immunogens obtained from microorganisms may be partially isolated from the microorganism so that two or more immunogens are present in the immunogenic composition. Similarly, immunogens obtained from a tumor cell may be partially isolated from the tumor cell so that two or more tumor associated antigens are present in the immunogenic composition.

With respect to immunogenic compositions comprising a recombinant expression vector, the nucleotide sequence may encode more than one immunogen, for example, at least two, three, four, five, or more immunogens (i.e., two, three, four, five, or more immunogens). In certain specific embodiments, each of the additional immunogens (e.g., second, third, etc immunogen) may induce an immune response to the same designated antigen as the first immunogen. In other specific embodiments, each of the additional immunogens (e.g., second, third, etc immunogen) may induce an immune response specific for a different (e.g., a second, third, etc.) designated antigen, respectively.

In particular embodiments, one immunogenic composition (also called a first immunogenic composition) that comprises the at least one isolated/recombinant immunogen (and which composition may further comprise an adjuvant) is capable of inducing a CD4 T cell response that is specific for the immunogen and thereby specific for the designated antigen and may also induce a humoral response (i.e., specific antibody response or antigen-specific antibody response) to the immunogen and the designated antigen. The other immunogenic composition (or second immunogenic composition) comprising the recombinant expression vector that comprises a nucleotide sequence encoding the immunogen is capable of inducing a CD8 T cell response specific for the immunogen and thus capable of inducing a CD8 T cell response specific for the designated antigen. As described in greater detail herein, the immunogen has one or more immunogenic regions that comprise epitope(s) that are capable of inducing a CD4 T cell response and a CD8 T cell response specific for the immunogen.

In other particular embodiments, immunogenic preparations are provided wherein the first immunogenic composition comprising at least one isolated/recombinant immunogen (called for convenience a first immunogen) may further comprise at least one additional isolated/recombinant immunogen. In other embodiments, the recombinant expression vector included in the second, different immunogenic composition may encode the first immunogen and encode at least one additional immunogen. In still other alternative embodiments, the first immunogenic composition comprises at least two isolated/recombinant immunogens and the second immunogenic composition comprises an expression vector that contains a nucleotide sequence that encodes the first immunogen and at least one additional immunogen.

In certain embodiments when induction of an immune response specific for two or more immunogens is desired, at least one immunogen is capable of inducing an immune response that comprises at least a specific humoral and/or CD4 T cell response and at least one additional immunogen is capable of inducing an immune response that comprises at least a specific CD8 T cell immune response. Accordingly, provided herein in one embodiment is an immunogenic preparation comprising (a) an immunogenic composition (which may be called a first immunogenic composition) that comprises a first isolated/recombinant immunogen (which composition may further comprise an adjuvant) and (b) a second immunogenic composition that comprises a recombinant expression vector that encodes and directs expression of the first immunogen and a second immunogen, wherein at least the second immunogen is capable of inducing a specific CD8 T cell response. In certain embodiments, each of the at least two immunogens has the capability to induce an immune response to the same designated antigen. Alternatively, each of the at least two immunogens has the capability to induce an immune response specific for a different designated antigen (for convenience, also called the first and second designated antigens, etc. respectively).

### Immunogens and Designated Antigens

An immunogen, which may be an isolated and/or recombinant immunogen included in one immunogenic composition and/or which is encoded by a recombinant expression vector contained within the second immunogenic composition, used in the methods and for the uses described herein includes any immunogen for which induction of a specific immune response is desired. In certain embodiments, the immunogen comprises an exemplary full-length amino acid sequence of a designated antigen of interest, or the immunogen may be an immunogenic fragment of the respective designated antigen. In other certain embodiments, an immunogen may comprise a variant of the designated antigen, which variant shares a high percent identity with an exemplary full-length designated antigen and exhibits substantially the same level of immunogenicity as the designated antigen comprising the exemplary amino acid sequence (i.e., the variant retains a level of immunogenicity to a statistically, clinically, and/or biologically significant degree compared with the immunogenicity of the exemplary or wild-type antigen). In particular, immunogens that immunogenic fragments or are variants of a designated antigen retain, in a statistically, clinically, or biologically significant manner, the capability to induce a humoral immune response (i.e., a B cell response resulting in expression of specific antibodies) or cell-mediated response (i.e., a CD4 T cell response and/or CD8 T cell response and including a cytotoxic T lymphocyte response)) or both a humoral and cell-mediated response in a subject. Designated antigens of interest and immunogenic fragments and immunogenic variants thereof are described in greater detail herein.

As described in greater detail herein, an immunogen comprises at least one immunogenic region or immunogenic epitope capable of inducing in a subject an immune response specific for a designated antigen. In one specific embodiment, the immunogen comprises one or more immunogenic regions such that the immunogen is capable of inducing any one or more of an antibody response, a CD4 T cell response, and a CD8 T cell response, wherein each response is specific for the immunogen and thus specific for the respective designated antigen. Accordingly, the immunogenic region comprises at least one epitope (i.e., one or more epitopes) that induces one or more of an antibody response, a CD4 T cell response, and a CD8 T cell response.

A cell-mediated immune response includes a cytotoxic T lymphocyte response, which response may destroy or damage a cell (e.g., a tumor cell, bacterial cell, virus, parasite, or fungal cell) or infectious particle (e.g., a virus particle) that produces or expresses the immunogen or the respective designated antigen. Any antigen that is associated with a disease or disorder for which a humoral response or cell-mediated immune response or both is beneficial to the immunized subject can be used as an immunogen.

Antigens associated with many diseases and disorders are well known in the art. An antigen of interest (i.e., a designated antigen) may be previously known to be associated with the disease or disorder, or may be identified as an antigen associated with a disease or disorder by any method known and practiced in the art. For example, an antigen associated with a type of cancer from which a patient is suffering may be known, such as a tumor-associated antigen, or may be identified from the tumor itself by any of a variety of methods practiced in the art. In certain embodiments, the designated antigen is a tumor-associated antigen (also called herein a tumor antigen) derived from a cancer cell (i.e., tumor cell), and one or more such tumor antigens may be useful for the immunotherapeutic treatment of cancers. By way of non-limiting example, tumor-associated antigens may be derived from prostate, breast, colorectal, lung, pancreatic, renal, mesothelioma, ovarian, or melanoma cancers. These and additional tumor-associated antigens are described herein and in the art.

In certain embodiments, immunogens include full-length proteins that are the designated antigens and are derived from a tumor or malignancy. In other certain embodiments, an immunogen comprises one or more immunogenic fragments that contain one or more epitopes from such proteins. In still other embodiments, an immunogen comprises a fusion polypeptide that comprises the full-length designated antigen or that comprises one, two, three, or more immunogenic fragments of the designated antigen derived from a tumor cell. In other embodiments, when an immunogenic composition is prepared for use in inducing an immune response against two or more designated antigens, a fusion polypeptide may comprise any combination of full-length antigen or one or more immunogenic fragments thereof for each of the two or more designated antigens. By way of example, a fusion polypeptide may comprise one or more immunogenic fragments obtained from one tumor associated antigen and may further comprise one or more immunogenic fragments obtained a second, different tumor associated antigen. Fusion proteins may comprise, in additional to the immunogenic polypeptide or peptide, at least one polypeptide or peptide, which is sometimes referred to as a carrier protein in the immunology art, that enhances the immune response to the immunogen of interest.

Exemplary tumor-associated antigens or tumor cell-derived antigens include MAGE 1, 3, and MAGE 4 (or other MAGE antigens such as those disclosed in International Patent Application Publication No. WO99/40188); PRAME; BAGE; RAGE, Lage (also known as NY ESO 1); SAGE; and HAGE (see, e.g., International Patent Application Publication No. WO 99/53061) or GAGE (Robbins et al., Curr. Opin. Immunol. 8:628-36 (1996); Van den Eynde et al., Int. J. Clin. Lab. Res. 27:81-86 (1997); Van den Eynde et al., Curr. Opin. Immunol. 9:648-93 (1997); Correale et al., J. Natl. Cancer Inst. 89: 293 (1997)). These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP) (Hubert et al., Proc. Natl. Acad. Sci. USA 96 14523-28, 1999); see also, e.g., Reiter et al., Proc. Nat. Acad. Sci. USA 95:1735-40, 1998; Nelson, et al., Proc. Natl. Acad. Sci. USA 96:3114-19 (1999); WO 98/12302; U.S. Pat. Nos. 5,955,306; 5,840,871 and 5,786,148; Int'l Patent Appl. Publication Nos. WO 98/20117; WO 00/04149; WO 98/137418).

Other tumor associated antigens include Plu-1 (J. Biol. Chem. 274:15633-45, 1999), HASH-1, HasH-2, Cripto (Salomon et al., Bioessays 199, 21:61-70; U.S. Pat. No. 5,654,140) and Criptin (U.S. Pat. No. 5,981,215). Additionally, a tumor antigen may be a self peptide hormone, such as whole length gonadotrophin hormone releasing hormone (GnRH, Int'l Patent Appl. Publication No. WO 95/20600), a short 10 amino acid long peptide, useful in the treatment of many cancers.

Tumor antigens include tumor antigens derived from cancers that are characterized by tumor associated antigen expression, such as HER-2/neu expression. Tumor associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1 MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, β-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RARα, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1α and HIF-2α), Nuclear Factor-Kappa B (NF-κB), Notch receptors (e.g., Notch1-4), c-Met, mammalian targets of rapamycin (mTOR), WNT proteins (a family of secreted lipid-modified signaling glycoproteins, including Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16), extracellular signal-regulated kinases (ERKs), and their regulatory subunits, PMSA, PR-3, MDM2, Mesothelin, renal cell carcinoma—5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGs5, SART3, STn, PAXS, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, and idiotype.

Immunogens also include tumor antigens that comprise epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that comprise epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein. See also Boon et al., Ann. Rev. Immunol. 12:337-65 (1994); Renkvist et al., Cancer Immunol. Immunother. 50:3-15 (2001).

In other embodiments, an immunogen is obtained or derived from a pathogenic microorganism or from an opportunistic pathogenic microorganism (also called herein an infectious disease microorganism), such as a virus, fungus, parasite, and bacterium. In certain embodiments, immunogens derived from such a microorganism include full-length proteins that are the selected designated antigens. In other certain embodiments, an immunogen comprises one or more immunogenic fragments that contain one or more epitopes from such proteins. In still other embodiments, an immunogen comprises a fusion polypeptide that comprises one, two, or more immunogenic fragments of a protein derived from a microorganism. In still other embodiments, an immunogen comprises a fusion polypeptide that comprises the full-length designated antigen or that comprises one, two, three, or more immunogenic fragments of the designated antigen derived from a microorganism. In other embodiments, when an immunogenic composition is prepared for use in inducing an immune response against two or more designated antigens of an infectious disease microorganism, a fusion polypeptide may comprise any combination of full-length antigen or one or more immunogenic fragments thereof for each of the two or more designated antigens. By way of example, a fusion polypeptide may comprise one or more immunogenic fragments obtained from one microbial antigen (i.e., a viral, bacterial, parasitic, or fungal antigen) and may further comprise one or more immunogenic fragments obtained a second, different microbial antigen (i.e., a second, different viral, bacterial, parasitic, or fungal antigen). Fusion proteins may comprise, in additional to the immunogenic polypeptide or peptide, at least one polypeptide or peptide, which is sometimes referred to as a carrier protein in the immunology art, that enhances the immune response to the immunogen of interest.

Illustrative pathogenic organisms whose antigens are contemplated as designated antigens and immunogens for use in the immunogenic compositions described herein and that are encoded by the vectors and vector particles described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as immunogens as described herein are known in the art and the amino acid sequences of such proteins (and species thereof) and nucleotide sequences encoding the proteins may be identified in publications and in public databases such as GENBANK, Swiss-Prot, and TrEMBL.

Antigens derived from human immunodeficiency virus (HIV) that may be immunogens and used as described herein include any of the HIV virion structural proteins (e.g., gp120, gp41, p17, p24), protease, reverse transcriptase, or HIV proteins encoded by tat, rev, nef, vif, vpr and vpu. HIV proteins and immunogenic fragments thereof are well known to the skilled person and may be found in any of a number of public databases (see, e.g., Vider-Shalit et al., AIDS 23(11):1311-18 (2009); Watkins, Mem. Inst. Oswaldo Cruz. 103(2):119-29 (2008); Gao et al., Expert Rev. Vaccines (4 Suppl):S161-68 (2004)). (See also, e.g., Klimstra et al., 2003. J. Virol. 77:12022-32; Bernard et al., Virology 276:93-103 (2000); Byrnes et al., J. Virol. 72: 7349-56 (1998); Lieberman et al., AIDS Res Hum. Retroviruses 13(5): 383-92 (1997); Menendez-Arias et al., Viral Immunol. 11(4): 167-181 (1998).

Antigens derived from herpes simplex virus (e.g., HSV 1 and HSV2) that are contemplated for use as immunogens in the compositions described herein and encoded by vectors and vector particles described herein include, but are not limited to, proteins expressed from HSV late genes. The late group of genes predominantly encodes proteins that form the virion particle. Such proteins include the five proteins from (U_{L}) which form the viral capsid: UL6, UL18, UL35, UL38 and the major capsid protein UL19, UL45, and UL27, each of which may be used as an immunogens as described herein (see, e.g., McGeoch et al., Virus Res. 117:90-104 (2006); Mettenleiter et al., Curr. Opin. Microbiol. 9: 423-29 (2006)). Other illustrative HSV proteins contemplated for use as immunogens herein include the ICP27 (H1, H2), glycoprotein B (gB) and glycoprotein D (gD) proteins. The HSV genome comprises at least 74 genes, each encoding a protein that could potentially be used as an immunogen to induce a T cell response (including a CTL response), B cell response, or both a CTL response and a B cell response.

Protective immune responses against HSV-2 in humans (see, e.g., Corey et al., “Genital Herpes,” in Sexually Transmitted Diseases, Holmes et al., eds. (McGraw-Hill, New York, 1999) 285-312) and in animal models (see, e.g., Parr et al., J. Virol. 72:2677 (1998)) suggest that an appropriate HSV-2 vaccine formulation is a desirable and obtainable objective. Over the past four decades, a series of HSV vaccine human trials using inactivated, whole HSV preparations and subunit HSV proteins formulated with adjuvants have been conducted in the United States and in Europe. Although moderate therapeutic efficacy with these vaccines was observed in some short-term studies, results from appropriately controlled trials with longer follow-up windows have been largely disappointing (see, e.g., Rajcani et al., Folia Microbiol. (Praha) 51:67 (2006)).

In Europe in the 1960s and 1970s, large clinical trials were conducted with formaldehyde-inactivated HSV (Eli Lilly trial) or heat-inactivated HSV (Lupidon H trial). Although improvements in the severity and frequency of HSV recurrences were reported, only a small subset of these trials was placebo-controlled and double-blinded. Furthermore, these vaccines did not confer long-term therapeutic efficacy. Maternal-fetal HSV-2 transmission studies in the 1980s demonstrated that infants of HSV-2 seropositive women possessed a lower risk of transmission versus women who acquired HSV-2 near term, suggesting that neutralizing antibodies (nAb) against the HSV-2 glycoproteins gD and gB may confer protection (see, e.g., Koelle et al., Clin. Microbiol. Rev. 16: 96 (2003)). Designed to generate nAb against these HSV-2 glycoprotein, trials by Glaxo-SmithKline and Chiron in the United States in the 1990s tested recombinant subunit vaccines with gD alone or with gB formulated with three different adjuvants: alum, MF-59 (oil-in-water), and monophosphoryl lipid A (MPL). These vaccines elicited or boosted HSV-2 specific nAb in seronegative individuals and cross-reactive nAb in HSV-1 seropositive individuals (see, e.g., Burke, Rev. Infect. Dis. 13 Suppl 11:S906-S911 (1991)). However, despite reaching target levels of humoral immunogenicity, these vaccines showed no therapeutic efficacy in men and only transient efficacy in women, suggesting that anti-HSV nAb are insufficient and that a successful HSV vaccine will likely need to generate potent T cell immunity (see, e.g., Corey et al., JAMA 282: 331 (1999); Stanberry, et al., N. Engl. J. Med. 347:1652 (2002)).

Previously conducted HSV vaccine trials indicates that nAb may not be sufficient to protect humans against HSV-2 infection, and data suggested that HSV-2-specific T cells play a critical role in reducing viral acquisition, transmission, and reactivation (see, e.g., Corey et al., JAMA (1999) supra). For example, individuals with deficiencies in T cell function have prolonged, more severe episodes of HSV-2 infection, and in longitudinal biopsy studies of HSV-2 lesions, viral clearance correlated with the infiltration of CD8 T cells (see, e.g., Koelle et al., J. Clin. Invest. 101:1500 (1998)). Additional HSV studies have shown that: type 1 helper T cell (Th1) responses were protective in animal models (see, e.g., Koelle, et al., J. Immunol. 166:4049 (2001); Zhu, et al., J. Exp. Med. 204:595 (2007)), the severity and frequency of HSV-2 reactivation was inversely correlated to the frequency of HSV-specific T cells, and infiltration of HSV-2 specific CTL into genital lesions correlated with viral clearance (see, e.g., Koelle et al., J. Infect. Dis. 169:956 (1994); Koelle et al., J. Clin. Invest. 110:537 (2002); Koelle et al., J. Clin. Invest. (1998) supra). These findings are consistent with data from subunit vaccine studies that implicated CD8 and Th1 CD4 T cell responses in mucosal HSV-2 clearance (see, e.g., Posavad et al., Nat. Med. 4:381 (1998)). Furthermore, HSV-2-specific CD8 T cells have been detected for long periods at the dermal-epidermal junction after resolution of genital herpes (see, e.g., Cattamanchi et al., Clin. Vaccine Immunol. 15:1638 (2008)).

Antigens derived from cytomegalovirus (CMV) that are contemplated for use in certain embodiments of the present immunogenic compositions and methods described herein include CMV structural proteins, viral antigens expressed during the immediate early and early phases of virus replication, glycoproteins I and III, capsid protein, coat protein, lower matrix protein pp65 (ppUL83), p52 (ppUL44), IE1 and IE2 (UL123 and UL122), protein products from the cluster of genes from UL128-UL150 (Rykman, et al., J. Virol. 2006 January; 80(2):710-22), envelope glycoprotein B (gB), gH, gN, and pp150. As would be understood by the skilled person, CMV proteins for use as immunogens described herein may be identified in public databases such as GenBank, Swiss-Prot, and TrEMBL (see e.g., Bennekov et al., Mt. Sinai J. Med. 71 (2): 86-93 (2004); Loewendorf et al., J. Intern. Med. 267(5):483-501 (2010); Marschall et al., Future Microbiol. 4:731-42 (2009)).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., Front. Biosci. 13:5916-27 (2008)).

Antigens derived from respiratory syncytial virus (RSV) that are contemplated for use as immunogens as described herein include any of the eleven proteins encoded by the RSV genome, or immunogenic fragments thereof: NS1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use as immunogens include any one of the five major proteins encoded by the VSV genome, and immunogenic fragments thereof: large protein (L), glycoprotein (G), nucleoprotein (N), phosphoprotein (P), and matrix protein (M) (see, e.g., Rieder et al., J. Interferon Cytokine Res. (2009) (9):499-509; Roberts et al., Adv. Virus Res. (1999) 53:301-19).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2. See e.g., Nature 437 (7062): 1162-66.

Examples of immunogens that are viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (as discussed herein and including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, bacterial antigens may be selected as designated antigens, and a bacterial antigen, or an immunogenic fragment or variant thereof, may be used as an immunogen. In certain embodiments, a bacterial antigen of interest may be a secreted polypeptide. In other certain embodiments, bacterial antigens that may be useful as immunogens for inducing an immune response include antigens that have a portion or portions of the polypeptide exposed on the outer cell surface of the bacteria. The portions of the polypeptide immunogens exposed on the cell surface are accessible to immune cells and/or antibodies in the host and thus may be useful immunogens encoded by the recombinant expression vectors and included in the immunogenic compositions comprising an immunogen (which may further comprise an adjuvant) described herein.

Antigens derived from Staphylococcus species including Methicillin-resistant Staphylococcus aureus (MRSA) that are contemplated for use as immunogens include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as immunogens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., Nucleic Acids Res. (2007) 35: 401-406). As would be understood by the skilled person, Staphylococcus proteins for use as immunogens may also be identified in other public databases such as GenBank, Swiss-Prot, and TrEMBL.

Antigens derived from Streptococcus pneumoniae that are contemplated for use as immunogens in certain embodiments described herein include pneumolysin, PspA, choline-binding protein A (CbpA), NanA, NanB, SpnHL, PavA, LytA, Pht, and pilin proteins (RrgA; RrgB; RrgC) Immunogenic proteins of Streptococcus pneumoniae are also known in the art and are contemplated for use as immunogens (see, e.g., Zysk et al., Infect. Immun. 2000 68(6):3740-43). The complete genome sequence of a virulent strain of Streptococcus pneumoniae has been sequenced (see, e.g., Tettelin H, et al., Science (2001) 293(5529):498-506) and, as would be understood by the skilled person, S. pneumoniae proteins for use in the compositions described herein may also be identified in other public databases such as GenBank, Swiss-Prot, and TrEMBL. Proteins of particular interest for immunogens according to the present disclosure include virulence factors and proteins predicted to be exposed at the surface of the pneumococci (see, e.g., Tettelin et al., supra; Frolet et al., BMC Microbiol. (2010) July 12; 10:190; Rigden, et al., Crit. Rev. Biochem. Mol. Biol. (2003) 38(2):143-68; Jedrzejas, Microbiol. Mol. Biol. Rev. (2001) 65(2):187-207).

Examples of bacterial antigens that may be used as immunogens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y. pestis F1 and V antigens).

Examples of fungal antigens that may be immunogens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including protective antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

Induction of an immune response, including either a humoral response (i.e., a B cell response) or a cell-mediated response (including a cytotoxic T lymphocyte (CTL) response) or both may also contribute to phagocytosis or killing of additional organisms such as Pseudomonas aeruginosa, Mycobacterium tuberculosis, M. leprae, and Listeria innocula. A CTL immune response contributes to killing of P. aeruginosa, M. tuberculosis, M. leprae, and L. innocula (see, e.g., Oykhman et al., J. Biomed. Biotechnol. (2010: 249482); published on-line Jun. 23, 2010). Accordingly, immunogens useful for the immunogenic compositions described herein and that may be encoded by the recombinant expression vectors and vector particles comprising the vectors may also be derived from these bacteria. The amino acid sequences of numerous polypeptides encoded by the bacterial genome of any one of the bacteria species and expressed by the bacteria can be readily identified in the art and in publicly available protein sequence data bases. (See also, e.g., Stover et al., Nature 406:959 (2000)).

Immunogens as described herein may be obtained or derived from fungi or parasites. Exemplary parasites that induce an immune response, including a CTL immune response, include Schistosoma mansoni, Entameoba histolytica, Toxoplasma gondii, and Plasmodium falciparum (see, e.g., Oykhman, supra). Accordingly, protein antigens derived or obtained from these parasites may be useful as immunogens to induce an immune response against the respective parasite. Immunogens may also be obtained or derived from species of fungus, including without limitation, Cryptococcus neoformans and Candida albicans (see, e.g., Oykhman, supra).

Polypeptides that comprise at least one immunogenic fragment of an immunogenic polypeptide (e.g., any of the tumor associated antigens or microbial antigens described herein and/or in the art) may be used as immunogens and encoded by the recombinant expression vectors described herein. An immunogenic fragment comprises at least one T cell epitope or at least one B cell epitope. The immunogenic fragment may consist of at least 6, 10, 15, 20, 30, 40, 50, 60, 70, 80, 90, 100, or more contiguous amino acids of an immunogenic polypeptide. The immunogenic fragment may comprise any number of contiguous amino acids between the aforementioned such that, for example, an immunogenic fragment is between about 6-10, 10-15, 15-20, 20-30, 30-40, 40-50, 50-60, 60-70, 70-80, 80-90, 90-100, or more contiguous amino acids of an immunogenic polypeptide. The immunogenic fragments may comprise a sufficient number of contiguous amino acids that form a linear epitope and/or may comprise a sufficient number of contiguous amino acids that permit the fragment to fold in the same (or sufficiently similar) three-dimensional conformation as the full-length polypeptide from which the fragment is derived to present a non-linear epitope or epitopes (also referred to in the art as conformational epitopes). Assays for assessing whether the immunogenic fragment folds into a conformation comparable to the full-length polypeptide include, for example, the ability of the protein to react with mono- or polyclonal antibodies that are specific for native or unfolded epitopes, the retention of other ligand-binding functions, and the sensitivity or resistance of the polypeptide fragment to digestion with proteases (see, e.g., Sambrook et al., Molecular Cloning: A Laboratory Manual, 3d ed., Cold Spring Harbor Laboratory Press, NY (2001)). Accordingly, by way of example, the three-dimensional conformation of a polypeptide fragment is sufficiently similar to the full-length polypeptide when the capability to bind and the level of binding of an antibody that specifically binds to the full-length polypeptide is substantially the same for the fragment as for the full-length polypeptide (i.e., the level of binding has been retained to a statistically, clinically, and/or biologically sufficient degree compared with the immunogenicity of the exemplary or wild-type full-length antigen).

Determination of the three-dimensional structures of a polypeptide, or immunogenic fragment thereof, of interest may be performed by routine methodologies to determine whether the immunogenic fragment retains the spatial positioning of the amino acids as found in the full-length polypeptide. See, for instance, Bradley et al., Science 309:1868-71 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007). Also available in the art are software tools, for example, PSORT or PSORT II, and Spscan (Wisconsin Sequence Analysis Package, Genetics Computer Group) that are useful for predicting transmembrane segments and membrane topology of polypeptides that are known or believed to traverse a cellular membrane (see, for example, Nakai et al., Trends Biochem. Sci. 24:34-36 (1999)).

Separately, or in combination with the above-described techniques, and given an exemplary amino acid sequence of a designated antigen of interest, a person skilled in the art can identify potential epitopes of the polypeptide antigen (see, e.g., Jameson and Wolf, Comput. Appl. Biosci. 4:181-86 (1988)). By way of another example, Hopp and Woods describe the hydrophilicity method, which is based on empirical demonstrations of the close correlation between the hydrophilicity of polypeptide regions and their antigenicity (see, e.g., Hopp, Pept. Res. 6:183-90 (1993); Hofmann et al., Biomed. Biochim. Acta 46:855-66 (1987)). Computer programs are also available for identifying B cell or T cell epitopes. A BASIC program called EPIPLOT predicts B-cell antigenic sites in proteins from their primary structures by calculating and plotting flexibility, hydrophilicity, and antigenicity profiles using 13 different scales (see, for example, Menendez et al., Comput. Appl. Biosci. 6:101-105 (1990)). See also, such as, Van Regenmortel, Methods: a companion to Methods in Enzymology, 9: 465-472 (1996); Pellequer et al., “Epitope predictions from the primary structure of proteins,” In Peptide antigens: a practical approach (ed. G. B. Wisdom), pp. 7-25; Oxford University Press, Oxford (1994); Van Regenmortel, “Molecular dissection of protein antigens” In Structure of antigens (ed. M. H. V. Van Regenmortel), Vol. 1, pp. 1-27. CRC Press, Boca Raton (1992).

T cell epitopes of a designated antigen that may be used as an immunogen may also be identified using a peptide motif searching program based on algorithms developed by Rammensee, et al. (Immunogenetics 50: 213-219 (1999)); by Parker, et al. (supra), or by using methods such as those described by Doytchinova and Flower in Immunol. Cell Biol. 80(3):270-9 (2002); Blythe et al., Bioinformatics 18:434-439 (2002); Guan et al., Applied Bioinformatics 2:63-66 (2003); Flower et al., Applied Bioinformatics 1:167-176 (2002); Mallios, Bioinformatics 17: 942-48 (2001); Schirle et al., J. Immunol. Meth. 257:1-16 (2001).

Epitopic regions of designated microbial antigens or designated tumor antigens that may be used as immunogens in the compositions and methods described herein are also described in the art. See by way of example, Lamb et al., Rev. Infect. Dis. March-April: Suppl 2:s443-447 (1989); Lamb et al., EMBO J. 6:1245-49 (1987); Lamb et al., Lepr. Rev. Suppl 2:131-37 (1986); Mehra et al., Proc. Natl. Acad. Sci. USA 83:7013-27 (1986); Horsfall et al., Immunol. Today 12:211-13 (1991); Rothbard et al., Curr. Top. Microbiol. Immunol. 155:143-52 (1990); Singh et al., Bioinformatics 17:1236-37 (2001); DeGroot et al., Vaccine 19:4385-95 (2001); DeLalla et al., J. Immunol. 163:1725-29 (1999); Cochlovius et al., J. Immunol. 165:4731-41 (2000); Consogno et al., Blood 101:1039-44 (2003); Roberts et al., AIDS Res. Hum. Retrovir. 12:593-610 (1996); Kwok et al., Trends Immunol. 22:583-88 (2001); Novak et al., J. Immunol. 166:6665-70 (2001).

Additional methods for identifying epitopic regions include methods described in Hoffmeister et al., Methods 29:270-281 (2003); Maecker et al., J. Immunol. Methods 255:27-40 (2001). Assays for identifying epitopes are described herein and known to the skilled artisan and include, for example, those described in Current Protocols in Immunology, Coligan et al. (Eds), John Wiley & Sons, New York, N.Y. (1991).

Identifying an immunogenic region and/or epitope of a designated antigen of interest can also be readily determined empirically by a person skilled in the art and/or by computer analysis and computer modeling, using methods and techniques that are routinely practiced by persons skilled in the art. Empirical methods include, by way of example, synthesizing polypeptide fragments comprising a particular length of contiguous amino acids of a protein, or generating fragments by use of one or more proteases and then determining the immunogenicity of the fragments using any one of numerous binding assays or immunoassay methods routinely practiced in the art. Exemplary methods for determining the capability of an antibody (polyclonal, monoclonal, or antigen-binding fragment thereof) to specifically bind to a fragment include, but are not limited to, ELISA, radioimmunoassay, immunoblot, competitive binding assays, fluorescence activated cell sorter analysis (FACS), and surface plasmon resonance.

Sequences of T cell and B cell epitopes can be obtained from publically available databases. For example, a peptide database that includes T-cell defined tumor antigens can be found on the Internet in a peptide database sponsored by Cancer Immunity (see cancerimmunity(dot)org/peptidedatabase/Tcellepitopes.htm), which is updated periodically. Another available database supported by the National Institute of Allergy and Infectious Diseases, which provides tools for searching for B cell and T cell epitopes and provides epitope analysis tools (see immune Epitope Database and Analysis Resource at immunoepitope(dot)org).

In certain instances when antigen-specific T cell lines or clones are available, for example tumor-infiltrating lymphocytes (TIL), virus-specific or bacteria-specific cytotoxic T lymphocytes (CTL), these cells may be used to screen for the presence of relevant epitopes using target cells prepared with specific antigens. Such targets can be prepared using random, or selected, synthetic peptide libraries, which would be used to sensitize the target cells for lysis by the CTL. Another approach to identify a relevant epitope when T cell lines or clones are available is to use recombinant DNA methodologies. Gene or cDNA libraries from CTL-susceptible targets are first prepared and transfected into non-susceptible target cells. This allows the identification and cloning of the gene encoding the protein precursor of the peptide containing the CTL epitope. The second step in this process is to prepare truncated genes from the relevant cloned gene, in order to narrow down the region that encodes for the at least one CTL epitope. This step is optional if the gene is not too large. The third step is to prepare synthetic peptides of, for example, approximately 10-20 amino acids in length, overlapping by 5-10 residues, which are used to sensitize targets for the CTL. When a peptide, or peptides, is shown to contain the relevant epitope, and if desired, smaller peptides can be prepared to establish the peptide of minimal size that contains the epitope. These epitopes are typically, but not necessarily, contained within 9-10 residues for CTL epitopes and up to 20 or 30 residues for helper T lymphocyte (HTL) epitopes.

Alternatively, epitopes may be defined by direct elution of peptides that are non-covalently bound by particular major histocompatibility complex (MHC) molecules followed by amino acid sequencing of the eluted peptides (see, for example, Engelhard et al., Cancer J. 2000 May; 6 Suppl 3:S272-80). Briefly, the eluted peptides are separated using a purification method such as HPLC, and individual fractions are tested for their capacity to sensitize targets for CTL lysis or to induce proliferation of cytokine secretion in HTL. When a fraction has been identified as containing the peptide, it is further purified and submitted to sequence analysis. The peptide sequence can also be determined using tandem mass spectrometry. A synthetic peptide is then prepared and tested with the CTL or HTL to corroborate that the correct sequence and peptide have been identified.

Epitopes may also be identified using computer analysis, such as the Tsites program (see, e.g., Rothbard and Taylor, EMBO J. 7:93-100, 1988; Deavin et al., Mol. Immunol. 33:145-155, 1996), which searches for peptide motifs that have the potential to elicit Th responses. CTL peptides with motifs appropriate for binding to murine and human class I or class II MHC may be identified according to BIMAS (Parker et al., J. Immunol. 152:163, 1994) and other HLA peptide binding prediction analyses. Briefly, the protein sequences, for example from microbial components or antigens, or tumor cell components or tumor antigens, are examined for the presence of MHC-binding motifs. These binding motifs, which exist for each MHC allele, are conserved amino acid residues, usually at positions 2 (or 3) and 9 (or 10) for MHC class I binding peptides that are typically 9-10 residues long. Synthetic peptides are then prepared that comprise those sequences bearing the MHC binding motifs, and subsequently such peptides are tested for their ability to bind to MHC molecules. The MHC binding assay can be carried out either using cells which express high numbers of empty (unoccupied) MHC molecules (cellular binding assay), or using purified MHC molecules. Lastly, the MHC binding peptides are then tested for their capacity to induce a CTL response in naive individuals, either in vitro using human lymphocytes, or in vivo using HLA-transgenic animals. These CTL are tested using peptide-sensitized target cells, and targets that naturally process the antigen, such as viral infected cells or tumor cells. To further confirm immunogenicity, a peptide may be tested using an HLA A2 transgenic mouse model and/or any of a variety of in vitro stimulation assays.

In certain embodiments, an immunogen includes polypeptide species of the designated antigen that have one or more amino acid substitutions, insertions, or deletions in an amino acid sequence that is known and available in the art for the respective immunogen. Conservative substitutions of amino acids are well known and may occur naturally in the polypeptide or may be introduced when the polypeptide is recombinantly produced. Amino acid substitutions, deletions, and additions may be introduced into a polypeptide using well-known and routinely practiced mutagenesis methods (see, e.g., Sambrook et al. Molecular Cloning: A Laboratory Manual, 3d ed., Cold Spring Harbor Laboratory Press, N Y 2001)). Oligonucleotide-directed site-specific (or segment specific) mutagenesis procedures may be employed to provide an altered polynucleotide that has particular codons altered according to the substitution, deletion, or insertion desired. Deletion or truncation variants of designated antigens that may be used as immunogens may also be constructed by using convenient restriction endonuclease sites adjacent to the desired deletion. Subsequent to restriction, overhangs may be filled in and the DNA re-ligated. Alternatively, random mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al., supra). Species (or variants) of a particular designated antigen (or polypeptide fragment thereof) include a polypeptide immunogen that has at least 85%, 90%, 95%, or 99% amino acid sequence identity to any of the exemplary amino acid sequences known in the art.

These polypeptide immunogen variants retain one or more biological activities or functions of the respective designated antigen. In particular, immunogens that are variants of a designated antigen retain, in a statistically, clinically, or biologically significant manner, the capability to induce an immune response (e.g., a humoral response (i.e., B cell response), cell-mediated response (i.e., T cell response (including a cytotoxic T lymphocyte response)) or both a humoral and cell-mediated response in a subject. Given the many molecular biology, protein expression, and protein isolation techniques and methods routinely practiced in the art for introducing mutations in a polypeptide, preparing polypeptide fragments, isolating the fragments and variants, and analyzing same, immunogen polypeptide variants and fragments having the desired biological activities can be made readily and without undue experimentation.

A variety of criteria known to persons skilled in the art indicate whether an amino acid that is substituted at a particular position in a peptide or polypeptide is conservative (or similar) For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e g, alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine) In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, the BLAST algorithm, or other algorithms described herein and practiced in the art).

As described herein for immunogenic fragments, assays for assessing whether a respective variant folds into a conformation comparable to the non-variant polypeptide or fragment include, for example, the ability of the protein to react with mono- or polyclonal antibodies that are specific for native or unfolded epitopes, the retention of ligand-binding functions, and the sensitivity or resistance of the mutant protein to digestion with proteases (see Sambrook et al., supra). Such variants can be identified, characterized, and/or made according to methods described herein or other methods known in the art, which are routinely practiced by persons skilled in the art.

Isolated/recombinant immunogens included in the immunogenic compositions described herein may be produced and prepared according to various methods and techniques routinely practiced in the molecular biology and/or polypeptide purification arts. Construction of an expression vector that is used for recombinantly producing an immunogen of interest can be accomplished using any of numerous suitable molecular biology engineering techniques known in the art, including, without limitation, the standard techniques of restriction endonuclease digestion, ligation, transformation, plasmid purification, and DNA sequencing, for example as described in Sambrook et al. (1989 and 2001 editions; Molecular Cloning: A Laboratory Manual, Cold Spring Harbor Laboratory Press, NY) and Ausubel et al. (Current Protocols in Molecular Biology (2003)). To obtain efficient transcription and translation, the polynucleotide sequence in each recombinant expression construct includes at least one appropriate expression control sequence (also called a regulatory sequence), such as a leader sequence and particularly a promoter operatively linked to the nucleotide sequence encoding the immunogen.

Host cells are genetically engineered with the recombinant expression vector to produce the immunogen(s), or fragments or variants thereof, by recombinant techniques. Each of the polypeptides and fusion polypeptides described herein can be expressed in mammalian cells, yeast, bacteria, insect, or other cells under the control of appropriate promoters. Cell-free translation systems can also be employed to produce such proteins using RNAs derived from DNA constructs. Appropriate cloning and expression vectors for use with prokaryotic and eukaryotic hosts are described, for example, by Sambrook, et al., Molecular Cloning: A Laboratory Manual, Third Edition, Cold Spring Harbor, N.Y., (2001).

Generally, recombinant expression vectors useful for producing an immunogen of interest include origins of replication, selectable markers permitting transformation of the host cell, for example, the ampicillin resistance gene of E. coli and S. cerevisiae TRP1 gene, and a promoter derived from a highly expressed gene to direct transcription of a downstream structural sequence. Promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences.

Optionally, a heterologous sequence can be inserted in frame with the nucleotide sequence that encodes the immunogen to provide a peptide or polypeptide that imparts desired characteristics, e.g., that simplifies purification of the expressed recombinant product. Such identification peptides include a polyhistidine tag (his tag) or FLAG® epitope tag, beta-galactosidase, alkaline phosphatase, GST, or the XPRESS™ epitope tag (Invitrogen Life Technologies, Carlsbad, Calif.) and the like (see, e.g., U.S. Pat. No. 5,011,912; Hopp et al., (Bio/Technology 6:1204 (1988)). The affinity sequence may be supplied by a vector, such as, for example, a hexa-histidine tag that is provided in pBAD/His (Invitrogen). Alternatively, the affinity sequence may be added either synthetically or engineered into the primers used to recombinantly generate the nucleic acid coding sequence (e.g., using the polymerase chain reaction).

Host cells containing described recombinant expression constructs may be genetically engineered (transduced, transformed, or transfected) with the expression constructs (for example, a cloning vector, a shuttle vector, or an expression construct). The vector or construct may be in the form of a plasmid, a viral particle, a phage, etc. The engineered host cells can be cultured in conventional nutrient media modified as appropriate for activating promoters, selecting transformants, or amplifying encoding-nucleotide sequences. Selection and maintenance of culture conditions for particular host cells, such as temperature, pH and the like, will be readily apparent to the ordinarily skilled artisan. Preferably the host cell can be adapted to sustained propagation in culture to yield a cell line according to art-established methodologies for production of polypeptides. In certain embodiments, the cell line is an immortal cell line, which refers to a cell line that can be repeatedly (at least ten times while remaining viable) passaged in culture following log-phase growth.

Useful bacterial expression constructs are constructed by inserting into an expression vector a structural DNA sequence encoding a desired immunogen together with suitable translation initiation and termination signals in operable reading phase with a functional promoter. The construct may comprise one or more phenotypic selectable markers and an origin of replication to ensure maintenance of the vector construct and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. Any other plasmid or vector may be used as long as they are replicable and viable in the host. Thus, for example, the nucleotide sequence that encodes an immunogen or designated antigen of interest may be included in any one of a variety of a recombinant expression constructs for expressing a polypeptide. Such vectors and constructs include chromosomal, nonchromosomal, and synthetic DNA sequences, e.g., bacterial plasmids; phage DNA; baculovirus; yeast plasmids; vectors derived from combinations of plasmids and phage DNA; viral DNA, such as vaccinia, adenovirus, fowl pox virus, and pseudorabies. However, any other vector may be used for preparation of a recombinant expression construct as long as it is replicable and viable in the host.

The appropriate DNA sequence(s) may be inserted into the vector by a variety of procedures. In general, the DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. Numerous standard techniques are described, for example, in Ausubel et al. (Current Protocols in Molecular Biology (Greene Publ. Assoc. Inc. & John Wiley & Sons, Inc., 2003)); Sambrook et al. (Molecular Cloning: A Laboratory Manual, 3rd Ed., (Cold Spring Harbor Laboratory 2001)); Maniatis et al. (Molecular Cloning, (Cold Spring Harbor Laboratory 1982)), and elsewhere.

The DNA sequence encoding a polypeptide immunogen in the expression vector is operatively linked to at least one appropriate expression control sequences (e.g., a promoter or a regulated promoter) to direct mRNA synthesis. Representative examples of such expression control sequences include LTR or SV40 promoter, the E. coli lac or trp, the phage lambda P_{L} promoter, and other promoters known to control expression of genes in prokaryotic or eukaryotic cells or their viruses. Promoter regions can be selected from any desired gene using CAT (chloramphenicol transferase) vectors or other vectors with selectable markers. Particular bacterial promoters include lacI, lacZ, T3, T5, T7, gpt, lambda P_{R}, P_{L}, and trp. Eukaryotic promoters include CMV immediate early, HSV thymidine kinase, early and late SV40, LTRs from retroviruses, and mouse metallothionein-I. Selection of the appropriate vector and promoter and preparation of certain recombinant expression constructs comprising at least one promoter or regulated promoter operatively linked to a nucleotide sequence that encodes an at least one immunogen is well within the level of ordinary skill in the art.

Design and selection of inducible, regulated promoters and/or tightly regulated promoters are known in the art and will depend on the particular host cell and expression system (see, e.g., E. coli arabinose operon (P_{BAD} or P_{ARA}) as described in Guzman et al., J. Bacteriology 177:4121-30 (1995); Smith et al., J. Biol. Chem. 253:6931-33 (1978); Hirsh et al., Cell 11:545-50 (1977); PET Expression Systems (see U.S. Pat. No. 4,952,496) available from Stratagene (La Jolla, Calif.); tet-regulated expression systems (Gossen et al., Proc. Natl. Acad. Sci. USA 89:5547-51 (1992); Gossen et al., Science 268:1766-69 (1995)); pLP-TRE2 Acceptor Vector (BD Biosciences Clontech, Palo Alto, Calif.) is designed for use with CLONTECH's Creator™ Cloning Kits); see also, e.g., Sauer, Methods 14:381-92 (1998); Furth, J. Mamm. Gland Biol. Neoplas. 2:373 (1997)); see, e.g., Cascio, Artif. Organs 25:529 (2001)).

The immunogen-encoding nucleic acid sequences may be cloned into a baculovirus shuttle vector, which is then recombined with a baculovirus to generate a recombinant baculovirus expression construct that is used to infect, for example, Sf9 host cells (see, e.g., Baculovirus Expression Protocols, Methods in Molecular Biology Vol. 39, Richardson, Ed. (Human Press 1995); Piwnica-Worms, “Expression of Proteins in Insect Cells Using Baculoviral Vectors,” Section II, Chapter 16 in Short Protocols in Molecular Biology, 2^{nd} Ed., Ausubel et al., eds., (John Wiley & Sons 1992)).

Methods that may be used for isolated and purifying a recombinant immunogen, by way of example, may include obtaining supernatants from suitable host/vector systems that secrete the recombinant immunogen into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen or designated antigen from its natural environment.

Methods for large scale production of one or more of the isolated/recombinant immunogens described herein include batch cell culture, which is monitored and controlled to maintain appropriate culture conditions. Purification of the immunogen may be performed according to methods described herein and known in the art and that comport with laws and guidelines of domestic and foreign regulatory agencies.

### Adjuvants and Adjuvant Compositions

As described herein, immunogenic compositions may further comprise at least one adjuvant that is intended to enhance (or improve, augment) the immune response to the immunogen and to its respective designated antigen (i.e., increase the level of the specific immune response to the immunogen and designated antigen in a statistically, biologically, or clinically significant manner compared with the level of the specific immune response in the absence of administering the adjuvant). In certain embodiments, an immunogenic composition comprises at least one immunogen, which may be isolated and/or recombinant, and at least one adjuvant.

In other certain embodiments, an immunogenic composition comprising a recombinant expression vector that encodes the at least one immunogen and is capable of directing expression of the immunogen further comprises an adjuvant. In other certain embodiments, both the immunogenic composition that comprises the at least one immunogen and the immunogenic composition comprising the recombinant expression vector further comprise an adjuvant. In still other embodiments, instead of combining an adjuvant with the immunogenic composition comprising the recombination expression vector or administering the adjuvant concurrently with this immunogenic composition, the adjuvant is administered at a later time and may be administered by a different route and/or a different site than the immunogenic composition comprising the vector. When the adjuvant is administered after administration of the immunogenic composition comprising the recombinant expression vector, the adjuvant is administered at 18 hours, 24 hours, 36 hours, 72 hours or 1 day, 2 days, 3 days, 4, days, 5 days, 6 days, or seven days (1 week) after administration of the immunogenic composition. Methods and techniques for determining the level of an immune response are discussed in greater detail herein and are routinely practiced in the art.

Exemplary adjuvants that may be included in the immunogenic compositions and used in the methods described herein include, but are not necessarily limited to, the following. Adjuvants that may be used in these methods include adjuvants useful for enhancing the humoral response, the cellular response, or both the humoral and cellular responses specific for the immunogen(s) and respective designated antigen(s). The cellular immune response comprises a CD4 T cell response (which may include a memory CD4 T cell response) and a CD8 T cell response specific for the immunogen and its respective designated antigen. The cellular response may also include a cytotoxic T cell response (CTL response) to the immunogen (or to a cell or particle bearing or expressing the immunogen(s)). Desired adjuvants augment the response to the immunogen without causing conformational changes in the immunogen that might adversely affect the qualitative form of the response. Suitable adjuvants include aluminum salts, such as alum (potassium aluminum sulfate), or other aluminum containing adjuvants; nontoxic lipid A-related adjuvants such as, by way of non-limiting example, nontoxic monophosphoryl lipid A (see, e.g., Tomai et al., J. Biol. Response Mod. 6:99-107 (1987); Persing et al., Trends Microbiol. 10:s32-s37 (2002)); GLA described herein; 3 De-O-acylated monophosphoryl lipid A (MPL) (see, e.g., United Kingdom Patent Application No. GB 2220211); adjuvants such as QS21 and QuilA that comprise a triterpene glycoside or saponin isolated from the bark of the Quillaja saponaria Molina tree found in South America (see, e.g., Kensil et al., in Vaccine Design: The Subunit and Adjuvant Approach (eds. Powell and Newman, Plenum Press, N Y, 1995); U.S. Pat. No. 5,057,540). Other suitable adjuvants include oil in water emulsions (such as squalene or peanut oil), optionally in combination with immune stimulants, such as monophosphoryl lipid A (see, e.g., Stoute et al., N. Engl. J. Med. 336, 86-91 (1997)). Another suitable adjuvant is CpG (see, e.g., Klinman, Int. Rev. Immunol. 25(3-4):135-54 (2006); U.S. Pat. No. 7,402,572; European Patent No. 772 619).

As described herein, a suitable adjuvant is an aluminum salt, such as aluminum hydroxide, aluminum phosphate, or aluminum sulfate. Such adjuvants can be used with or without other specific immunostimulating agents such as MPL or 3-DMP, QS21, polymeric or monomeric amino acids such as polyglutamic acid or polylysine. Another class of suitable adjuvants is oil-in-water emulsion formulations (also called herein stable oil in water emulsions). Such adjuvants can be optionally used with other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™) or other bacterial cell wall components. Oil-in-water emulsions include (1) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.); (2) SAF, containing 10% Squalane, 0.4% Tween 80, 5% pluronic-blocked polymer L121, and thr-MDP, either microfluidized into a submicron emulsion or vortexed to generate a larger particle size emulsion, and (3) Ribi adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™). Also as described above, suitable adjuvants include saponin adjuvants, such as Stimulon™ (QS21, Aquila, Worcester, Mass.) or particles generated therefrom such as ISCOMs (immunostimulating complexes) and ISCOMATRIX. Other adjuvants include Complete Freund's Adjuvant (CFA) (which is suitable for non-human use but is unsuitable for human use) and Incomplete Freund's Adjuvant (IFA). Other adjuvants include cytokines, such as interleukins (IL-1, IL-2, and IL-12), macrophage colony stimulating factor (M-CSF), and tumor necrosis factor (TNF).

As described herein, an adjuvant may be a non-toxic lipid A-related (or lipid A derivative) adjuvant. In a particular embodiment, an adjuvant is selected on the basis of its capability to act as a Toll-like receptor (TLR) agonist. By way of example, a non-toxic lipid A-related adjuvant that acts as a TLR4 agonist and that may be used in the compositions described herein is identified as DSLP. DSLP compounds share the features that they contain a disaccharide (DS) group formed by the joining together of two monosaccharide groups selected from glucose and amino substituted glucose, where the disaccharide is chemically bound to both a phosphate (P) group and to a plurality of lipid (L) groups. More specifically, the disaccharide may be visualized as being formed from two monosaccharide units, each having six carbons. In the disaccharide, one of the monosaccharides will form a reducing end, and the other monosaccharide will form a non-reducing end. For convenience, the carbons of the monosaccharide forming the reducing terminus will be denoted as located at positions 1, 2, 3, 4, 5 and 6, while the corresponding carbons of the monosaccharide forming the non-reducing terminus will be denoted as being located at positions 1′, 2′, 3′, 4′, 5′ and 6′, following conventional carbohydrate numbering nomenclature. In the DSLP, the carbon at the 1 position of the non-reducing terminus is linked, through either an ether (—O—) or amino (—NH—) group, to the carbon at the 6′ position of the reducing terminus. The phosphate group will be linked to the disaccharide, preferably through the 4′ carbon of the non-reducing terminus. Each of the lipid groups will be joined, through either amide (—NH—C(O)—) or ester (—O—C(O)—) linkages to the disaccharide, where the carbonyl group joins to the lipid group. The disaccharide has 7 positions that may be linked to an amide or ester group, namely, positions 2′, 3′, and 6′ of the non-reducing terminus, and positions 1, 2, 3 and 4 of the reducing terminus.

A lipid group has at least six carbons, preferably at least 8 carbons, and more preferably at least 10 carbons, where in each case the lipid group has no more than 24 carbons, no more than 22 carbons, or no more than 20 carbons. In one embodiment, the lipid groups taken together provide 60-100 carbons, preferably 70 to 90 carbons. A lipid group may consist solely of carbon and hydrogen atoms, i.e., it may be a hydrocarbyl lipid group, or it may contain one hydroxyl group, i.e., it may be a hydroxyl-substituted lipid group, or it may contain an ester group which is, in turn, joined to a hydrocarbyl lipid or a hydroxyl-substituted lipid group through the carbonyl (—C(O)—) of the ester group, i.e., a ester substituted lipid. A hydrocarbyl lipid group may be saturated or unsaturated, where an unsaturated hydrocarbyl lipid group will have one double bond between adjacent carbon atoms.

The DSLP comprises 3, or 4, or 5, or 6 or 7 lipid groups. In one aspect, the DSLP comprises 3 to 7 lipid groups, while in another aspect the DSLP comprises 4-6 lipids. In one aspect, the lipid group is independently selected from hydrocarbyl lipid, hydroxyl-substituted lipid, and ester substituted lipid. In one aspect, the 1, 4′ and 6′ positions are substituted with hydroxyl. In one aspect, the monosaccharide units are each glucosamine. The DSLP may be in the free acid form, or in the salt form, e.g., an ammonium salt.

In certain embodiments, the lipid on the DSLP is described by the following: the 3′ position is substituted with —O—(CO)—CH2-CH(Ra)(—O—C(O)—Rb); the 2′ position is substituted with —NH—(CO)—CH2-CH(Ra)(—O—C(O)—Rb); the 3 position is substituted with —O—(CO)—CH2-CH(OH)(Ra); the 2 position is substituted with —NH—(CO)—CH2-CH(OH)(Ra); where each of Ra and Rb is selected from decyl, undecyl, dodecyl, tridecyl, tetradecyl, wherein each of these terms refer to saturated hydrocarbyl groups. In one embodiment, Ra is undecyl and Rb is tridecyl, where this adjuvant is described in, for example, U.S. Patent Application Publication 2008/0131466 as “GLA.” The compound wherein Ra is undecyl and Rb is tridecyl may be used in a stereochemically defined form, as available from, for example, Avanti Polar Lipid as PHAD™ adjuvant.

In one aspect, the DSLP is a mixture of naturally-derived compounds known as 3D-MPL. 3D-MPL adjuvant is produced commercially in a pharmaceutical grade form by GlaxoSmithKline Company as their MPL™ adjuvant. 3D-MPL has been extensively described in the scientific and patent literature, see, e.g., Vaccine Design: the subunit and adjuvant approach, Powell M. F. and Newman, M. J. eds., Chapter 21 Monophosphoryl Lipid A as an adjuvant: past experiences and new directions by Ulrich, J. T. and Myers, K. R., Plenum Press, New York (1995) and U.S. Pat. No. 4,912,094.

In another aspect, the DSLP adjuvant may be described as comprising (i) a diglucosamine backbone having a reducing terminus glucosamine linked to a non-reducing terminus glucosamine through an ether linkage between hexosamine position 1 of the non-reducing terminus glucosamine and hexosamine position 6 of the reducing terminus glucosamine; (ii) an O-phosphoryl group attached to hexosamine position 4 of the non-reducing terminus glucosamine; and (iii) up to six fatty acyl chains; wherein one of the fatty acyl chains is attached to 3-hydroxy of the reducing terminus glucosamine through an ester linkage, wherein one of the fatty acyl chains is attached to a 2-amino of the non-reducing terminus glucosamine through an amide linkage and comprises a tetradecanoyl chain linked to an alkanoyl chain of greater than 12 carbon atoms through an ester linkage, and wherein one of the fatty acyl chains is attached to 3-hydroxy of the non-reducing terminus glucosamine through an ester linkage and comprises a tetradecanoyl chain linked to an alkanoyl chain of greater than 12 carbon atoms through an ester linkage. See, e.g., U.S. Patent Application Publication No. 2008/0131466.

In another aspect, the adjuvant may be a synthetic disaccharide having six lipid groups as described in U.S. patent application publication 2010/0310602.

In another aspect, a DSLP adjuvant is described by chemical formula (I) and is referred to as glucopyranosyl lipid A (GLA):

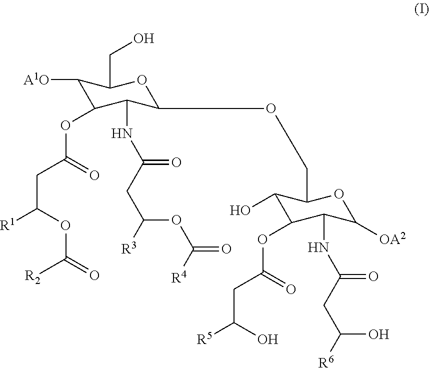

wherein the moieties A1 and A2 are independently selected from the group of hydrogen, phosphate, and phosphate salts. Sodium and potassium are exemplary counterions for the phosphate salts. The moieties R¹, R², R³, R⁴, R⁵, and R⁶ are independently selected from the group of hydrocarbyl having 3 to 23 carbons, represented by C₃-C₂₃. For added clarity it will be explained that when a moiety is “independently selected from” a specified group having multiple members, it should be understood that the member chosen for the first moiety does not in any way impact or limit the choice of the member selected for the second moiety. The carbon atoms to which R¹, R³, R⁵ and R⁶ are joined are asymmetric, and thus may exist in either the R or S stereochemistry. In one embodiment all of those carbon atoms are in the R stereochemistry, while in another embodiment all of those carbon atoms are in the S stereochemistry. “Hydrocarbyl” refers to a chemical moiety formed entirely from hydrogen and carbon, where the arrangement of the carbon atoms may be straight chain or branched, noncyclic or cyclic, and the bonding between adjacent carbon atoms may be entirely single bonds, that is, to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl,” respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like. The adjuvant of formula (I) may be obtained by synthetic methods known in the art, for example, the synthetic methodology disclosed in PCT International Publication No. WO 2009/035528, which is incorporated herein by reference, as well as the publications identified in WO 2009/035528, each of which publications is also incorporated herein by reference. Certain of the adjuvants may also be obtained commercially.

The DSLP adjuvant may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. A chemically synthesized DSLP adjuvant, e.g., the adjuvant of formula (I), can be prepared in substantially homogeneous form, which refers to a preparation that is at least 80%, at least 85%, at least 90%, at least 95% or at least 96%, 97%, 98% or 99% pure with respect to the DSLP molecules present, e.g., the compounds of formula (I). Determination of the degree of purity of a given adjuvant preparation can be readily made by those familiar with the appropriate analytical chemistry methodologies, such as by gas chromatography, liquid chromatography, mass spectroscopy and/or nuclear magnetic resonance analysis. DSLP adjuvants obtained from natural sources are typically not easily made in a chemically pure form, and thus synthetically prepared adjuvants are preferred adjuvants for use in the compositions and methods described herein. As discussed previously, certain of the adjuvants may be obtained commercially. One such DSLP adjuvant is Product No. 699800 as identified in the catalog of Avanti Polar Lipids, Alabaster Ala., see E1 in combination with E10, below.

In various embodiments, the adjuvant has the chemical structure of formula (I) but the moieties A₁, A₂, R¹, R², R³, R⁴, R⁵, and R⁶ are selected from subsets of the options previously provided for these moieties, wherein these subsets are identified below by E1, E2, etc.

E1: A₁ is phosphate or phosphate salt and A₂ is hydrogen.
E2: R¹, R³, R⁵ and R⁶ are C₃-C₂₁ alkyl; and R² and R⁴ are C₅-C₂₃ hydrocarbyl.
E3: R¹, R³, R⁵ and R⁶ are C₅-C₁₇ alkyl; and R² and R⁴ are C₇-C₁₉ hydrocarbyl.
E4: R¹, R³, R⁵ and R⁶ are C₇-C₁₅ alkyl; and R² and R⁴ are C₉-C₁₇ hydrocarbyl.
E5: R¹, R³, R⁵ and R⁶ are C₉-C₁₃ alkyl; and R² and R⁴ are C₁₁-C₁₅ hydrocarbyl.
E6: R¹, R³, R⁵ and R⁶ are C₉-C₁₅ alkyl; and R² and R⁴ are C₁₁-C₁₇ hydrocarbyl.
E7: R¹, R³, R⁵ and R⁶ are C₇-C₁₃ alkyl; and R² and R⁴ are C₉-C₁₅ hydrocarbyl.
E8: R¹, R³, R⁵ and R⁶ are C₁₁-C₂₀ alkyl; and R² and R⁴ are C₁₂-C₂₀ hydrocarbyl.
E9: R¹, R³, R⁵ and R⁶ are C₁₁ alkyl; and R² and R⁴ are C₁₃ hydrocarbyl.
E10: R¹, R³, R⁵ and R⁶ are undecyl and R² and R⁴ are tridecyl.

In certain embodiments, each of E2 through E10 is combined with embodiment E1, and/or the hydrocarbyl groups of E2 through E9 are alkyl groups, preferably straight chain alkyl groups. The DSLP adjuvant, e.g., the adjuvant of formula (I) may be formulated into a pharmaceutical composition, optionally with a co-adjuvant, each as discussed below. In this regard reference is made to U.S. Patent Publication No. 2008/0131466 that provides formulations, such as aqueous formulation (AF) and stable emulsion formulations (SE) for GLA adjuvant, wherein these formulations may be used for any of the adjuvants of formula (I). In certain specific embodiments, an immunogenic composition comprises GLA wherein the GLA adjuvant (see formula I) is formulated in a stable oil-in water emulsion (SE) (GLA/SE or GLA-SE) and then combined with at least one immunogen.

Optionally, as described in greater detail below and herein, two or more different adjuvants can be used simultaneously, such as by way of non-limiting example, an aluminum salt with a DSLP adjuvant, an aluminum salt with QS21, a DSLP adjuvant with QS21, and alumna aluminum salt, QS21, and MPL or GLA together. Also, Incomplete Freund's adjuvant can be used (see, e.g., Chang et al., Advanced Drug Delivery Reviews 32, 173-186 (1998)), optionally in combination with any of an aluminum salt, QS21, and MPL and all combinations thereof.

In certain embodiments, the DSLP adjuvant, e.g., the adjuvant of formula (I), may be formulated into a pharmaceutical (or adjuvant composition), optionally with a co-adjuvant, each as discussed below or any other adjuvant described herein or available in the art. In this regard reference is made to U.S. Patent Publication No. 2008/0131466 that provides formulations, such as aqueous formulation (AF) and stable emulsion formulations (SE) for GLA adjuvant, which formulations may be used with respect to any of the adjuvants of formula (I).

As provided herein the DSLP adjuvant, such as the adjuvant of formula I, may be used in combination with a second adjuvant, referred to herein as a co-adjuvant. In three exemplary embodiments, the co-adjuvant may be a delivery system, or it may be an immunopotentiator, or it may be a composition that functions as both a delivery system and an immunopotentiator (see, e.g., O'Hagan et al., Pharm. Res. 21(9):1519-30 (2004)). The co-adjuvant may be an immunopotentiator that operates via a member of the Toll-like receptor family biomolecules. For example, the co-adjuvant may be selected for its primary mode of action, as either a TLR4 agonist, or a TLR8 agonist, or a TLR9 agonist. Alternatively, or in supplement, the co-adjuvant may be selected for its carrier properties; for example, the co-adjuvant may be an emulsion, a liposome, a microparticle, or alum.

In one embodiment, the co-adjuvant is alum, where this term refers to aluminum salts, such as aluminum phosphate (AlPO₄) and aluminum hydroxide (Al(OH)₃). When alum is used as the co-adjuvant, the alum may be present in a dose of an immunogenic composition (or preparation comprising the immunogenic composition) in an amount of about 100 to 1,000 μg, or 200 to 800 μg, or 300 to 700 μg or 400 to 600 μg. The adjuvant of formula (1) is typically present in an amount less than the amount of alum, and in various specific embodiments the adjuvant of formula (1), on a weight basis, is present at 0.1-1%, or 1-5%, or 1-10%, or 1-100% relative to the weight of alum.

In one particular embodiment, the adjuvant is an emulsion having adjuvanating properties sufficient for use in a vaccine or immunogenic composition. Such emulsions include oil-in-water emulsions. Freund's incomplete adjuvant (IFA) is one such adjuvant. Another suitable oil-in-water emulsion is MF-59™ adjuvant, which contains squalene, polyoxyethylene sorbitan monooleate (also known as Tween™ 80 surfactant), and sorbitan trioleate. Squalene is a natural organic compound originally obtained from shark liver oil, although also available from plant sources (primarily vegetable oils), including amaranth seed, rice bran, wheat germ, and olives. Other suitable adjuvants are Montanide™ adjuvants (Seppic Inc., Fairfield N.J.) including Montanide™ ISA 50V, which is a mineral oil-based adjuvant; Montanide™ ISA 206; and Montanide™ IMS 1312. While mineral oil may be present in the co-adjuvant, in one embodiment the oil component(s) of the immunogenic compositions described herein are all metabolizable oils.

Examples of immunopotentiators that may be used in the practice of the methods described herein as co-adjuvants include: MPL™; MDP and derivatives; oligonucleotides; double-stranded RNA; alternative pathogen-associated molecular patterns (PAMPS); saponins; small-molecule immune potentiators (SMIPs); cytokines; and chemokines.

In one embodiment, the co-adjuvant is MPL™ adjuvant, which is commercially available from GlaxoSmithKline (originally developed by Ribi ImmunoChem Research, Inc. Hamilton, Mont.). See, e.g., Ulrich and Myers, Chapter 21 from Vaccine Design: The Subunit and Adjuvant Approach, Powell and Newman, eds. Plenum Press, New York (1995). Related to MPL™ adjuvant, and also suitable as co-adjuvants for use in the compositions and methods described herein, are AS02™ adjuvant and AS04™ adjuvant. AS02™ adjuvant is an oil-in-water emulsion that contains both MPL™ adjuvant and QS-21™ adjuvant (a saponin adjuvant discussed elsewhere herein). AS04™ adjuvant contains MPL™ adjuvant and alum. MPL™ adjuvant is prepared from lipopolysaccharide (LPS) of Salmonella minnesota R595 by treating LPS with mild acid and base hydrolysis followed by purification of the modified LPS.

In another embodiment, the co-adjuvant is a saponin such as those derived from the bark of the Quillaja saponaria tree species, or a modified saponin (see, e.g., U.S. Pat. Nos. 5,057,540; 5,273,965; 5,352,449; 5,443,829; and 5,560,398). The product QS-21™ adjuvant sold by Antigenics, Inc. Lexington, Mass. is an exemplary saponin-containing co-adjuvant that may be used with the adjuvant of formula (I). An alternative co-adjuvant, related to the saponins, is the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

In yet another embodiment, the co-adjuvant is a cytokine that functions as a co-adjuvant (see, e.g., Lin et al., Clin. Infect. Dis. 21(6):1439-49 (1995); Taylor, Infect. Immun. 63(9):3241-44 (1995); and Egilmez, Chap. 14 in Vaccine Adjuvants and Delivery Systems, John Wiley & Sons, Inc. (2007)). In various embodiments, the cytokine may be, for example, granulocyte-macrophage colony-stimulating factor (GM-CSF) (see, e.g., Change et al., Hematology 9(3):207-15 (2004); Dranoff, Immunol. Rev. 188:147-54 (2002); and U.S. Pat. No. 5,679,356); or an interferon, such as a type I interferon (e.g., interferon-α (IFN-α) or interferon-β (IFN-β)), or a type II interferon (e.g., interferon-γ (IFN-γ) (see, e.g., Boehm et al., Ann. Rev. Immunol. 15:749-95 (1997); and Theofilopoulos et al., Ann. Rev. Immunol. 23:307-36 (2005)); an interleukin, specifically including interleukin-1α (IL-1α), interleukin-1β (IL-1β), interleukin-2 (IL-2) (see, e.g., Nelson, J. Immunol. 172(7):3983-88 (2004); interleukin-4 (IL-4), interleukin-7 (IL-7), interleukin-12 (IL-12) (see, e.g., Portielje et al., Cancer Immunol. Immunother. 52(3):133-44 (2003); and Trinchieri, Nat. Rev. Immunol. 3(2):133-46 (2003)); interleukin-15 (Il-15), interleukin-18 (IL-18); fetal liver tyrosine kinase 3 ligand (Flt3L), or tumor necrosis factor α (TNFα). The DSLP adjuvant, such as the adjuvant of formula (I), may be co-formulated with the cytokine prior to combination with the vaccine antigen, or the antigen, DSLP adjuvant (e.g., adjuvant of formula (I)), and cytokine co-adjuvant may be formulated separately and then combined.

In certain embodiments, an immunogenic composition that comprises an immunogen (which may be isolated and/or recombinant) and an adjuvant are formulated together. In other certain embodiments, when the immunogenic composition comprises two or more immunogens, an adjuvant may be formulated with each immunogen separately or the two or more immunogens may be formulated together with an adjuvant to form a single immunogenic composition. When two or more immunogens are intended to be administered to a subject and when each immunogen is separately formulated with an adjuvant, each composition may then be combined to form a single immunogenic composition.

In other certain embodiments, an immunogenic composition comprising the immunogen or a composition comprising a recombinant expression vector that encodes the immunogen or a vector particle comprising the vector are packaged and supplied in separate vials than those containing the adjuvant. Each of the immunogenic compositions and adjuvant may be combined with a pharmaceutically acceptable (i.e., physiologically suitable or acceptable) excipient(s), which are described in greater detail herein. Appropriate labels are typically packaged with each composition indicating the intended therapeutic application. The choice of an adjuvant and/or the excipient depends on the stability of the immunogen, recombinant expression vector, and/or vector particle; the route of administration; the dosing schedule; and the efficacy of the adjuvant for the species being vaccinated. For administration in humans, a pharmaceutically acceptable adjuvant is one that has been approved or is approvable for human administration by pertinent regulatory bodies. For example, as discussed herein and known in the art, Complete Freund's adjuvant is not suitable for human administration.

Adjuvants useful for use in the immunological compositions and methods described herein are physiologically or pharmaceutically suitable adjuvants for the subject to whom the adjuvant is administered. Adjuvant compositions comprise at least one adjuvant (i.e., one or more adjuvants) and, optionally, at least one physiologically or pharmaceutically suitable (or acceptable) excipient. Any physiological or pharmaceutically suitable excipient or carrier (i.e., a non-toxic material that does not interfere with the activity of the active ingredient) known to those of ordinary skill in the art for use in pharmaceutical compositions may be employed in the adjuvant compositions described herein. Exemplary excipients include diluents and carriers that maintain stability and integrity of the component(s) of the adjuvant. Excipients for therapeutic use are well known, and are described, for example, in Remington: The Science and Practice of Pharmacy (Gennaro, 21^{st} Ed. Mack Pub. Co., Easton, Pa. (2005)), and are described in greater detail herein.

### Recombinant Expression Vectors

In one embodiment, recombinant expression vectors are provided that comprise a polynucleotide sequence encoding at least one immunogen that induces an immune response to the immunogen and to its respective designated antigen. To obtain efficient transcription and translation of the immunogen, the encoding polynucleotide sequences in each vector include at least one appropriate expression control sequence (also called a regulatory expression sequence or feature) (e.g., promoter, enhancer, leader), which are described in greater detail herein, that is operatively linked to the encoding polynucleotide sequence(s). These recombinant expression vectors are thus provided for directing expression of the immunogen or for directing co-expression of at least two immunogens in any appropriate host cell that has been transformed, transduced, or transfected with the recombinant expression vector or vector particle containing the recombinant expression vector.

The recombinant expression vectors described herein may encode one or more immunogens (i.e., at least one, at least two, at least three immunogens, etc.), which immunogens are described in greater detail herein. In particular embodiments, at least one, two, or three, or more immunogens from an infectious microorganism (e.g., a virus, bacteria, fungus, or parasite) may be encoded by a recombinant expression vector. Immunogens and designated antigens obtained from infectious disease microorganisms are described in greater detail herein. By way of example, an immunogen may be an HSV-2 protein, such as UL19 or gD, (or an immunogenic variant thereof) or may be an immunogenic fragment or region of the HSV-2 protein. In another specific embodiment, a recombinant expression vector described herein may encode at least one, two, three, or more tumor-associated antigens, or immunogenic variants or fragments thereof. These tumor associated antigens are described in greater detail herein and may be, for example, a tumor-associated antigen from a renal cell carcinoma antigen, a prostate cancer antigen (e.g., prostatic acid phosphatase, prostate specific antigen, NKX3.1, and prostate specific membrane antigen), a mesothelioma antigen, a pancreatic cancer antigen, a melanoma antigen, a breast cancer antigen, a colorectal cancer antigen, a lung cancer antigen, an ovarian cancer antigen, or any cancer or tumor-associate antigen described herein and in the art.

Recombinant expression vectors may be used for expression of any one or more of the immunogens described herein. In particular embodiments, the recombinant expression vector is delivered to an appropriate cell (for example, an antigen-presenting cell i.e., a cell that displays a peptide/MHC complex on its cell surface, such as a dendritic cell) or tissue (e.g., lymphoid tissue) that will induce the desired immune response (i.e., a specific humoral response (i.e., B cell response) and/or induction of a specific cell-medicated immune response, which may include an immunogen-specific CD4 and/or CD8 T cell response, which CD8 T cell response may include a cytotoxic T cell (CTL) response). The recombinant expression vectors may therefore also include, for example, lymphoid tissue-specific transcriptional regulatory elements (TRE) such as a B lymphocyte, T lymphocyte, or dendritic cell specific TRE. Lymphoid tissue specific TRE are known in the art (see, e.g., Thompson et al., Mol. Cell. Biol. 12, 1043-53 (1992); Todd et al., J. Exp. Med. 177, 1663-74 (1993); Penix et al., J. Exp. Med. 178:1483-96 (1993)).

In a particular embodiment, the recombinant expression vector is plasmid DNA or cosmid DNA. Plasmid DNA or cosmid DNA containing one or more polynucleotides encoding an immunogen as described herein is readily constructed using standard techniques well known in the art. The vector genome may be typically constructed in a plasmid form that can then be transfected into a packaging or producer cell line. The plasmid generally comprises sequences useful for replication of the plasmid in bacteria. Such plasmids are well known in the art. In addition, vectors that include a prokaryotic origin of replication may also include a gene whose expression confers a detectable or selectable marker such as a drug resistance. Typical bacterial drug resistance products are those that confer resistance to ampicillin or tetracycline. For analysis to confirm that the correct nucleotide sequences are incorporated in plasmids, the plasmid may be replicated in E. coli, purified, and analyzed by restriction endonuclease digestion and/or its nucleotide sequence determined by conventional methods.

In other particular embodiments, the recombinant expression vector is a viral vector. Exemplary recombinant expression viral vectors include a lentiviral vector genome, poxvirus vector genome, vaccinia virus vector genome, adenovirus vector genome, adenovirus-associated virus vector genome, herpes virus vector genome, and alpha virus vector genome. Viral vectors may be live, attenuated, replication conditional or replication deficient, and typically is a non-pathogenic (defective), replication competent viral vector.

By way of example, in a specific embodiment, when the viral vector is a vaccinia virus vector genome, the polynucleotide encoding an immunogen of interest may be inserted into a non-essential site of a vaccinia viral vector. Such non-essential sites are described, for example, in Perkus et al., Virology 152:285 (1986); Hruby et al., Proc. Natl. Acad. Sci. USA 80:3411 (1983); Weir et al., J. Virol. 46:530 (1983). Suitable promoters for use with vaccinia viruses include but are not limited to P7.5 (see, e.g., Cochran et al., J. Virol. 54:30 (1985); P11 (see, e.g., Bertholet, et al., Proc. Natl. Acad. Sci. USA 82:2096 (1985)); and CAE-1 (see, e.g., Patel et al., Proc. Natl. Acad. Sci. USA 85:9431 (1988)). Highly attenuated strains of vaccinia are more acceptable for use in humans and include Lister, NYVAC, which contains specific genome deletions (see, e.g., Guerra et al., J. Virol. 80:985-98 (2006); Tartaglia et al., AIDS Research and Human Retroviruses 8:1445-47 (1992)), or MVA (see, e.g., Gheradi et al., J. Gen. Virol. 86:2925-36 (2005); Mayr et al., Infection 3:6-14 (1975)). See also Hu et al. (J. Virol. 75:10300-308 (2001), describing use of a Yaba-Like disease virus as a vector for cancer therapy); U.S. Pat. Nos. 5,698,530 and 6,998,252. See also, e.g., U.S. Pat. No. 5,443,964. See also U.S. Pat. Nos. 7,247,615 and 7,368,116.

In certain embodiments, an adenovirus vector or adenovirus-associated virus vector may be used for expressing an immunogen of interest. Several adenovirus vector systems and methods for administering the vectors have been described (see, e.g., Molin et al., J. Virol. 72:8358-61 (1998); Narumi et al., Am J. Respir. Cell Mol. Biol. 19:936-41 (1998); Mercier et al., Proc. Natl. Acad. Sci. USA 101:6188-93 (2004); U.S. Pat. Nos. 6,143,290; 6,596,535; 6,855,317; 6,936,257; 7,125,717; 7,378,087; 7,550,296).

Retroviral vector genomes may include those based upon murine leukemia virus (MuLV), gibbon ape leukemia virus (GaLV), ecotropic retroviruses, simian immunodeficiency virus (SIV), human immunodeficiency virus (HIV), and combinations (see, e.g., Buchscher et al., J. Virol. 66:2731-39 (1992); Johann et al., J. Virol. 66:1635-40 (1992); Sommerfelt et al., Virology 176:58-59 (1990); Wilson et al., J. Virol. 63:2374-78 (1989); Miller et al., J. Virol. 65:2220-24 (1991); Miller et al., Mol. Cell Biol. 10:4239 (1990); Kolberg, NIH Res. 4:43 1992; Cornetta et al., Hum. Gene Ther. 2:215 (1991)).

In a more specific embodiment, the recombinant expression viral vector is a lentiviral vector genome. The genome can be derived from any of a large number of suitable, available lentiviral genome based vectors, including those identified for human gene therapy applications (see, e.g., Pfeifer et al., Annu. Rev. Genomics Hum. Genet. 2:177-211 (2001)). Suitable lentiviral vector genomes include those based on Human Immunodeficiency Virus (HIV-1), HIV-2, feline immunodeficiency virus (FIV), equine infectious anemia virus, Simian Immunodeficiency Virus (SIV), and maedi/visna virus. A desirable characteristic of lentiviruses is that they are able to infect both dividing and non-dividing cells, although target cells need not be dividing cells or be stimulated to divide. Generally, the genome and envelope glycoproteins will be based on different viruses, such that the resulting viral vector particle is pseudotyped. Safety features of the vector genome are desirably incorporated. Safety features include self-inactivating LTR and a non-integrating genome. Exemplary vectors contain a packaging signal (psi), a Rev-responsive element (RRE), splice donor, splice acceptor, central poly-purine tract (cPPT), and WPRE element. In certain exemplary embodiments, the viral vector genome comprises sequences from a lentivirus genome, such as the HIV-1 genome or the SIV genome. The viral genome construct may comprise sequences from the 5′ and 3′ LTRs of a lentivirus, and in particular may comprise the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Typically, the LTR sequences are HIV LTR sequences.

The vector genome may comprise an inactivated or self-inactivating 3′ LTR (see, e.g., Zufferey et al., J. Virol. 72: 9873, 1998; Miyoshi et al., J. Virol. 72:8150, 1998; both of which are incorporated in their entirety). A self-inactivating vector generally has a deletion of the enhancer and promoter sequences from the 3′ long terminal repeat (LTR), which is copied over into the 5′ LTR during vector integration. In one instance, the U3 element of the 3′ LTR contains a deletion of its enhancer sequence, the TATA box, Sp1 and NF-kappa B sites. As a result of the self-inactivating 3′ LTR, the provirus that is generated following entry and reverse transcription will comprise an inactivated 5′ LTR. The rationale is to improve safety by reducing the risk of mobilization of the vector genome and the influence of the LTR on nearby cellular promoters. The self-inactivating 3′ LTR may be constructed by any method known in the art.

Optionally, the U3 sequence from the lentiviral 5′ LTR may be replaced with a promoter sequence in the viral construct, such as a heterologous promoter sequence. This can increase the titer of virus recovered from the packaging cell line. An enhancer sequence may also be included. Any enhancer/promoter combination that increases expression of the viral RNA genome in the packaging cell line may be used. In one example, the CMV enhancer/promoter sequence is used (see, e.g., U.S. Pat. Nos. 5,385,839 and 5,168,062).

In certain embodiments, the risk of insertional mutagenesis is minimized by constructing the lentiviral vector genome to be integration defective. A variety of approaches can be pursued to produce a non-integrating vector genome. These approaches entail engineering a mutation(s) into the integrase enzyme component of the pol gene, such that it encodes a protein with an inactive integrase. The vector genome itself can be modified to prevent integration by, for example, mutating or deleting one or both attachment sites, or making the 3′ LTR-proximal polypurine tract (PPT) non-functional through deletion or modification. In addition, non-genetic approaches are available; these include pharmacological agents that inhibit one or more functions of integrase. The approaches are not mutually exclusive, that is, more than one of them can be used at a time. For example, both the integrase and attachment sites can be non-functional, or the integrase and PPT site can be non-functional, or the attachment sites and PPT site can be non-functional, or all of them can be non-functional.

Integrase is involved in cleavage of viral double-stranded blunt-ended DNA and joining the ends to 5′-phosphates in the two strands of a chromosomal target site. Integrase has three functional domains: N-terminal domain, which contains a zinc-binding motif (HHCC); the central domain core, which contains the catalytic core and a conserved DD35E motif (D64, D116, E152 in HIV-1); and a C-terminal domain, which has DNA binding properties. Point mutations introduced into integrase are sufficient to disrupt normal function. Many integrase mutations have been constructed and characterized (see, e.g., Philpott and Thrasher, Human Gene Therapy 18:483, 2007; Apolonia, Thesis submitted to University College London, April 2009, pp, 82-97; Engelman et al., J. Virol. 69: 2729, 1995; Nightingale et al., Mol. Therapy, 13: 1121, 2006). The sequence encoding the integrase protein can be deleted or mutated to render the protein inactive, preferably without significantly impairing reverse transcriptase activity or nuclear targeting, thereby only preventing integration of the provirus into the target cell genome. Acceptable mutations can reduce integrase catalysis, strand transfer, binding to att sites, binding to host chromosomal DNA, and other functions. For example, a single aspartic acid to asparagine substitution at residue 35 of HIV or SIV integrase completely abolishes viral DNA integration. Deletions of integrase will generally be confined to the C-terminal domain. Deletion of coding sequence for residues 235-288 result in a useful non-functional integrase (see, e.g., Engelman et al., J. Virol. 69:2729, 1995). As further examples, mutations can be generated, for example, Asp64 (residue numbers are given for HIV-1, corresponding residue numbers for integrase from other lentiviruses or retroviruses can be readily determined by one of ordinary skill) (e.g., D64E, D64V), Asp116 (e.g., D116N), Asn120 (e.g., N120K), Glu152, Gln148 (e.g., Q148A), Lys156, Lys159, Trp235 (e.g., W235E), Lys264 (e.g., K264R), Lys266 (e.g., K266R), Lys273 (e.g., K273R). Other mutations can be constructed and tested for integration, transgene expression, and any other desirable parameter. Assays for these functions are well known. Mutations can be generated by any of a variety of techniques, including site-directed mutagenesis and chemical synthesis of nucleic acid sequence. One mutation may be made or more than one of these mutations can be present in integrase. For example, an integrase may have mutations at two amino acids, three amino acids, four amino acids, and so on.

Alternatively or in combination with the use of integrase mutant(s), the attachment sites (att) in U3 and U5 can also be mutated. Integrase binds to these sites and the 3′-terminal dinucleotide is cleaved at both ends of the vector genome. A CA dinucleotide is located at the recessed 3′ end; the CA is required for processing, mutation of the nucleotides blocks integration into the host chromosome. The A of the CA dinucleotide is the most critical nucleotide for integration, and mutations at both ends of the genome will give the best results (see, e.g., Brown et al., J. Virol. 73:9011 (1999)). In one exemplification, the CA at each end is changed to TG. In other exemplifications, the CA at each end is changed to TG at one end and GT at the other end. In other exemplifications, the CA at each end is deleted; in other exemplifications, the A of the CA is deleted at each end.

Integration can also be inhibited by mutation or deletion of polypurine tract (PPT) (see, e.g., WO 2009/076524), located proximally to the 3′ LTR. The PPT is a polypurine sequence of about 15 nucleotides that can serve as a primer binding site for plus-strand DNA synthesis. In this instance, mutations or deletions of PPT targets the reverse transcription process. Without wishing to be held to a particular mechanism, by mutating or deleting PPT, production of linear DNA is radically reduced, and essentially only 1-LTR DNA circles are produced. Integration requires a linear double-stranded DNA vector genome, and integration is essentially eliminated without it. As stated herein, a PPT can be made non-functional by mutation or by deletion. Typically, the entire about 15 nt PPT is deleted, although in some embodiments, shorter deletions of 14 nt, 13, nt, 12 nt, 11 nt, 10 nt, 9 nt, 8 nt, 7 nt, 6 nt, 5 nt, 4 nt, 3 nt and 2 nt may be made. When mutations are made, typically multiple mutations are made, especially in the 5′ half of the PPT (see, e.g., McWilliams et al., J. Virol. 77:11150, 2003), although single and double mutations in the first four bases still reduce transcription. Mutations made at the 3′ end of PPT generally have a more dramatic effect (see, e.g., Powell et al., J. Virol. 70:5288, 1996).

These different approaches to make a vector genome non-integrating can be used individually or in combination. Using more than one approach may be used to build a fail-safe vector through redundant mechanisms. Thus, PPT mutations or deletions can be combined with att site mutations or deletions or with Integrase mutations or PPT mutations or deletions can be combined with both att site mutations or deletions and Integrase mutations. Similarly, att site mutations or deletions and Integrase mutations may be combined with each other or with PPT mutations or deletions.

As described herein, lentiviral vector constructs contain a promoter for expression in mammalian cells. Promoters, which are discussed in greater detail herein, include, for example, the human ubiquitin C promoter (UbiC), the cytomegalovirus immediate early promoter (CMV), and the Rous sarcoma virus (RSV) promoter. The U3 region may comprise a PPT (polypurine tract) sequence immediately upstream. In certain specific embodiments, any one of at least three different U3 regions (at the 3′ end) may be included in the lentiviral vector (see SEQ ID NOS:21-23 in US20120328655). The constructs contain deletions in the U3 regions. The SIN construct has a deletion of about 130 nucleotides in the U3 (see, e.g., Miyoshi, et al. J. Virol. 72: 8150, 1998; Yu et al., Proc. Natl. Acad. Sci. USA 83: 3194, 1986), which removes the TATA box, thereby abolishing LTR promoter activity. The deletions in constructs 703 and 704 increase expression from lentivirus vectors (see, e.g., Bayer et al., Mol. Therapy 16: 1968, 2008). In addition, construct 704 contains a deletion of the 3′ PPT, which decreases integration of the vector (see, e.g., WO 2009/076524). See also U.S. patent application Ser. No. 12/842,609 and International Patent Application Publication No. WO 2011/011584 (International Patent Application No. PCT/US10/042870), which are each incorporated by reference in their entirety.

Regulatory Expression Sequences

As described herein, the recombinant expression vector comprises at least one regulatory expression sequence. In certain embodiments, when the recombinant expression vector comprises a viral vector genome, expression of the at least one immunogen is desired in particular target cells. Typically, for example, in a lentiviral vector the polynucleotide sequence encoding the immunogen is located between the 5′ LTR and 3′ LTR sequences. Further, the encoding nucleotide sequence(s) is preferably operatively linked in a functional relationship with other genetic or regulatory sequences or features, for example transcription regulatory sequences including promoters or enhancers, that regulate expression of the immunogen in a particular manner. In certain instances, the useful transcriptional regulatory sequences are those that are highly regulated with respect to activity, both temporally and spatially. Expression control elements that may be used for regulating the expression of the encoded polypeptides are known in the art and include, but are not limited to, inducible promoters, constitutive promoters, secretion signals, enhancers, and other regulatory sequences.

The polynucleotide encoding the immunogen and any other expressible sequence is typically in a functional relationship with internal promoter/enhancer regulatory sequences. With respect to lentiviral vector constructs, an “internal” promoter/enhancer is one that is located between the 5′ LTR and the 3′ LTR sequences in the viral vector and is operatively linked to the encoding polynucleotide sequence of interest. The internal promoter/enhancer may be any promoter, enhancer or promoter/enhancer combination known to increase expression of a gene with which it is in a functional relationship. A “functional relationship” and “operatively linked” mean, without limitation, that the sequence is in the correct location and orientation with respect to the promoter and/or enhancer such that the sequence of interest will be expressed when the promoter and/or enhancer is contacted with the appropriate molecules.

The choice of an internal promoter/enhancer is based on the desired expression pattern of the immunogen and the specific properties of known promoters/enhancers. Thus, the internal promoter may be constitutively active. Non-limiting examples of constitutive promoters that may be used include the promoter for ubiquitin (see, e.g., U.S. Pat. No. 5,510,474; WO 98/32869); CMV (see, e.g., Thomsen et al., Proc. Natl. Acad. Sci. USA 81:659, 1984; U.S. Pat. No. 5,168,062); beta-actin (Gunning et al. 1989 Proc. Natl. Acad. Sci. USA 84:4831-4835); and pgk (see, for example, Adra et al. 1987 Gene 60:65-74; Singer-Sam et al. 1984 Gene 32:409-417; and Dobson et al. 1982 Nucleic Acids Res. 10:2635-2637).

Alternatively, the promoter may be a tissue specific promoter. In some embodiments, the promoter is a target cell-specific promoter. For example, the promoter can be from any product expressed by dendritic cells, including CD11c, CD103, TLRs, DC-SIGN, BDCA-3, DEC-205, DCIR2, mannose receptor, Dectin-1, Clec9A, MHC class II. In addition, promoters may be selected to allow for inducible expression of the immunogen. A number of systems for inducible expression are known in the art, including the tetracycline responsive system, the lac operator-repressor system, as well as promoters responsive to a variety of environmental or physiological changes, including heat shock, metal ions, such as metallothionein promoter, interferons, hypoxia, steroids, such as progesterone or glucocorticoid receptor promoter, radiation, such as VEGF promoter. A combination of promoters may also be used to obtain the desired expression of each of the immunogen-encoding polynucleotide sequences. The artisan of ordinary skill will be able to select a promoter based on the desired expression pattern of the polynucleotide sequence in the organism or the target cell of interest.

A recombinant expression vector, including a viral vector genome, may comprise at least one RNA Polymerase II or III responsive promoter. This promoter can be operatively linked to the polynucleotide sequence of interest and can also be linked to a termination sequence. In addition, more than one RNA Polymerase II or III promoter may be incorporated. RNA polymerase II and III promoters are well known to persons of skill in the art. A suitable range of RNA polymerase III promoters can be found, for example, in Paule and White, Nucleic Acids Res., Vol. 28, pp 1283-1298 (2000). RNA polymerase II or III promoters also include any synthetic or engineered DNA fragment that can direct RNA polymerase II or III to transcribe downstream RNA coding sequences. Further, the RNA polymerase II or III (Pol II or III) promoter or promoters used as part of the viral vector genome can be inducible. Any suitable inducible Pol II or III promoter can be used with the methods described herein. Particularly suited Pol II or III promoters include the tetracycline responsive promoters provided in Ohkawa and Taira, Human Gene Therapy, 11:577-585 (2000) and in Meissner et al., Nucleic Acids Res., 29:1672-1682 (2001).

An internal enhancer may also be present in the recombinant expression vector, including a viral vector genome, to increase expression of the polynucleotide sequence of interest. For example, the CMV enhancer (see, e.g., Boshart et al., Cell 41:521, 1985) may be used. Many enhancers in viral genomes, such as HIV, CMV, and in mammalian genomes have been identified and characterized (see, e.g., publically available databases such as GenBank). An enhancer can be used in combination with a heterologous promoter. One of ordinary skill in the art will be able to select the appropriate enhancer based on the desired expression pattern.

When targeting delivery of a recombinant expression vector, including a viral vector genome, to a particular target cell, the vector genome will usually contain a promoter that is recognized by the target cell and that is operatively linked to the sequence of interest, viral components (when the vector is a viral vector), and other sequences discussed herein. A promoter is an expression control element formed by a nucleic acid sequence that permits binding of RNA polymerase and transcription to occur. Promoters may be inducible, constitutive, temporally active or tissue specific. The activity of inducible promoters is induced by the presence or absence of biotic or abiotic factors. Inducible promoters can be a useful tool in genetic engineering because the expression of genes to which they are operatively linked can be turned on or off at certain stages of development of an organism, its manufacture, or in a particular tissue. Inducible promoters can be grouped as chemically-regulated promoters, and physically-regulated promoters. Typical chemically-regulated promoters include, not are not limited to, alcohol-regulated promoters (e.g., alcohol dehydrogenase I (alcA) gene promoter), tetracycline-regulated promoters (e.g., tetracycline-responsive promoter), steroid-regulated promoter (e.g., rat glucocorticoid receptor (GR)-based promoter, human estrogen receptor (ER)-based promoter, moth ecdysone receptor-based promoter, and the promoters based on the steroid/retinoid/thyroid receptor superfamily), metal-regulated promoters (e.g., metallothionein gene-based promoters), and pathogenesis-related promoters (e.g., Arabidopsis and maize pathogen-related (PR) protein-based promoters). Typical physically-regulated promoters include, but are not limited to, temperature-regulated promoters (e.g., heat shock promoters), and light-regulated promoters (e.g., soybean SSU promoter). Other exemplary promoters are described elsewhere, for example, in patents and published patent applications that can be identified by searching the U.S. Patent and Trademark Office databases.

One of skill in the art will be able to select an appropriate promoter based on the specific circumstances. Many different promoters are well known in the art, as are methods for operatively linking the promoter to the polynucleotide sequence to be expressed. Both native promoter sequences and many heterologous promoters may be used to direct expression in the packaging cell and target cell. Heterologous promoters are typically used because they generally permit greater transcription and higher yields of the desired protein as compared to the native promoter.

The promoter may be obtained, for example, from the genomes of viruses such as polyoma virus, fowlpox virus, adenovirus, bovine papilloma virus, avian sarcoma virus, cytomegalovirus, a retrovirus, hepatitis-B virus, and Simian Virus 40 (SV40). The promoter may also be, for example, a heterologous mammalian promoter, for example, the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system. In some embodiments, the promoter is a dendritic cell-specific promoter. The dendritic cell-specific promoter can be, for example, CD11c promoter.

Transcription may be increased by inserting an enhancer sequence into the vector(s). Enhancers are typically cis-acting elements of DNA, usually about 10 to 300 base pairs in length, that act on a promoter to increase its transcription. Many enhancer sequences are now known from mammalian genes (globin, elastase, albumin, alpha-fetoprotein, and insulin) and from eukaryotic cell viruses. Examples include the SV40 enhancer on the late side of the replication origin (base pair 100-270), the cytomegalovirus early promoter enhancer, the polyoma enhancer on the late side of the replication origin, and adenovirus enhancers. The enhancer may be spliced into the vector at a position 5′ or 3′ to the antigen-specific polynucleotide sequence, but is preferably located at a site 5′ from the promoter.

Expression vectors may also contain sequences necessary for the termination of transcription and for stabilizing the mRNA. These sequences are often found in the 5′ and, occasionally 3′, untranslated regions of eukaryotic or viral DNAs or cDNAs and are well known in the art.

A recombinant expression construction, including a viral vector genome, may also contain additional genetic elements. The types of elements that may be included in the construct are not limited in any way and may be chosen to achieve a particular result. For example, a signal that facilitates nuclear entry of the recombinant expression vector or viral genome in the target cell may be included. An example of such a signal is the HIV-1 flap signal. Additional regulatory sequences may be included that facilitate the characterization of the provirus integration site in the target cell. For example, a tRNA amber suppressor sequence may be included in the construct. An insulator sequence, for example from chicken β-globin, may also be included in the viral genome construct. This element reduces the chance of silencing an integrated provirus in the target cell due to methylation and heterochromatinization effects. In addition, the insulator may shield the internal enhancer, promoter and exogenous polynucleotide sequences from positive or negative positional effects from surrounding DNA at the integration site on the chromosome. In addition, the recombinant construct, including the vector genome, may contain one or more genetic elements designed to enhance expression of the gene of interest. For example, a woodchuck hepatitis virus responsive element (WRE) may be placed into the construct (see, e.g., Zufferey et al. 1999. J. Virol. 74:3668-81; Deglon et al., 2000. Hum. Gene Ther. 11:179-90).

When the recombinant expression vector is a viral vector genome, the viral vector genome is typically constructed in a plasmid form that may be transfected into a packaging or producer cell line for production of the viral vector genome construct. The plasmid generally comprises sequences useful for replication of the plasmid in bacteria. Such plasmids are well known in the art. In addition, vectors that include a prokaryotic origin of replication may also include a gene whose expression confers a detectable or selectable marker such as a drug resistance. Typical bacterial drug resistance products are those that confer resistance to ampicillin or tetracycline.

In certain configurations, recombinant expression vectors contain polynucleotide sequences that encode one or more stimulatory or immunomodulatory factors. Exemplary stimulatory or immunomodulatory molecules include GM-CSF, IL-2, IL-4, IL-6, IL-7, IL-15, IL-21, IL-23, TNFα, B7.1, B7.2, 4-1BB, CD40 ligand (CD40L), drug-inducible CD40 (iCD40), and the like. These polynucleotides are typically under the control of one or more regulatory elements that direct the expression of the coding sequences in dendritic cells. In certain other particular embodiments, a recombinant expression vector is included that directs expression of and includes a nucleotide sequence that encodes both an immunogen and an immunomodulatory or immunostimulatory factor. In one embodiment, a recombinant expression vector is included that directs expression of and includes a nucleotide sequence that encodes both an immunogen and for example GM-CSF.

Maturation of dendritic cells contributes to successful vaccination (see, e.g., Banchereau et al., Nat. Rev. Immunol. 5:296-306 (2005); Schuler et al., Curr. Opin. Immunol. 15:138-147 (2003); Figdor et al., Nat. Med. 10:475-480 (2004)). Maturation can transform DCs from cells actively involved in antigen capture into cells specialized for T cell priming. For example, engagement of CD40 by CD40L on CD4-helper T cells is a critical signal for DC maturation, resulting in potent activation of CD8+ T cells. Such stimulatory molecules may also be referred to as maturation factors or maturation stimulatory factors.

Immune checkpoints represent significant barriers to activation of functional cellular immunity in cancer, and antagonistic antibodies specific for inhibitory ligands on T cells including CTLA4 and programmed death-1 (PD-1) are examples of targeted agents being evaluated in the clinics. A significant tolerance mechanism in chronic infections and cancer is the functional exhaustion of antigen-specific T cells that express high levels of PD-1. As the potency of therapeutic immunization has been shown to be significantly enhanced by combination with immune checkpoint control, as a non-limiting example, it can be appreciated by those of ordinary skill in the art that an alternative approach to inhibiting immune checkpoint is to inhibit the expression or activity of programmed death (PD) ligands one and two (PD-L1/L2). One way to accomplish inhibition is by the expression of RNA molecules such as those described herein, which repress the expression of PD-L1/L2 in the DCs transduced with a viral vector genome, such as the lentivirus vector genome, encoding one or more of the relevant molecules. Maturation of DCs or expression of particular elements such as immune checkpoints, for example PD-1 ligands, can be characterized by flow cytometry analysis of up-regulation of surface marker such as MHC II, and by profiling expressed chemokines and cytokines, for example, by performing techniques and methods described herein.

A sequence encoding a detectable product, usually a protein, can be included to allow for identification of cells that are expressing the desired immunogen. For example, a fluorescent marker protein, such as green fluorescent protein (GFP), is incorporated into the recombinant expression construct along with a polynucleotide sequence of interest (i.e., encoding an at least one immunogen). In other instances, the protein may be detectable by an antibody, or the protein may be an enzyme that acts on a substrate to yield a detectable product, or may be a protein product that allows selection of a transfected or transduced target cell, for example confers drug resistance, such as hygromycin resistance. Typical selection genes encode proteins that confer resistance to antibiotics or other toxins suitable for use in eukaryotic cells, for example, neomycin, methotrexate, blasticidine, among others known in the art, or complement auxotrophic deficiencies, or supply critical nutrients withheld from the media. The selectable marker can optionally be present on a separate plasmid and introduced by co-transfection.

With respect to vector particles described herein, one or more multicistronic expression units may be used that include two or more of a polynucleotide sequence encoding an immunogen, and a sequence encoding an envelope molecule as described herein or one or more DC maturation factors, immunomodulatory or stimulatory factors necessary for production of the desired vector particle in packaging cells. The use of multicistronic vectors reduces the total number of nucleic acid molecules required and thus may avoid the possible difficulties associated with coordinating expression from multiple vector genomes. In a multicistronic vector the various elements to be expressed are operatively linked to one or more promoters (and other expression control elements as necessary). In some configurations, a multicistronic vector comprises a sequence encoding at least one immunogen (i.e., one or more) of interest, a sequence encoding a reporter product, and a sequence encoding one or more vector particle components. In certain embodiments in which the recombinant construct comprises a polynucleotide that encodes an immunogen, the construct optionally encodes a DC maturation factor. In certain other embodiments, a multicistronic vector comprises a polynucleotide sequences that encode each of an immunogen, a DC maturation factor, and optionally viral components when the expression vector is a viral expression vector. In still other embodiments, multicistronic vectors direct expression and encode at least two or more immunogens.

Each component to be expressed in a multicistronic expression vector may be separated, for example, by an internal ribosome entry site (IRES) element or a viral 2A element, to allow for separate expression of the various proteins from the same promoter. IRES elements and 2A elements are known in the art (see, e.g., U.S. Pat. No. 4,937,190; de Felipe et al. 2004. Traffic 5: 616-626). In one embodiment, oligonucleotides such as furin cleavage site sequences (RAKR) (see, e.g., Fang et al. 2005 Nat. Biotech. 23: 584-590) linked with 2A-like sequences from foot-and-mouth diseases virus (FMDV); equine rhinitis A virus (ERAV); and thosea asigna virus (TaV) (see, e.g., Szymczak et al. 2004 Nat. Biotechnol. 22: 589-594) are used to separate genetic elements in a multicistronic vector. The efficacy of a particular multicistronic vector can readily be tested by detecting expression of each of the genes using standard protocols.

In a specific exemplification, a viral vector genome comprises: a cytomegalovirus (CMV) enhancer/promoter sequence; the R and U5 sequences from the HIV 5′ LTR; a packaging sequence (ψ); the HIV-1 flap signal; an internal enhancer; an internal promoter; a gene of interest; the woodchuck hepatitis virus responsive element; a tRNA amber suppressor sequence; a U3 element with a deletion of its enhancer sequence; the chicken β-globin insulator; and the R and U5 sequences of the 3′ HIV LTR. In some exemplifications, the vector genome comprises an intact lentiviral 5′ LTR and a self-inactivating 3′ LTR (see, e.g., Iwakuma et al. Virology 15:120, 1999).

Construction of the vector genome can be accomplished using any suitable genetic engineering techniques known in the art, including, without limitation, the standard techniques of restriction endonuclease digestion, ligation, transformation, plasmid purification, and DNA sequencing, for example as described in Sambrook et al. (1989 and 2001 editions; Molecular Cloning: A Laboratory Manual, Cold Spring Harbor Laboratory Press, NY); Coffin et al. (Retroviruses. Cold Spring Harbor Laboratory Press, N.Y. (1997)); and “RNA Viruses: A Practical Approach” (Alan J. Cann, Ed., Oxford University Press, (2000), each of the foregoing which is incorporated herein by reference in its entirety.

Vectors constructed for transient expression in mammalian cells may also be used. Transient expression involves the use of an expression vector that is able to replicate efficiently in a host cell, such that the host cell accumulates many copies of the expression vector and, in turn, synthesizes high levels of a the polypeptide encoded by the immunogen-specific polynucleotide in the expression vector. See Sambrook et al., supra, pp. 16.17-16.22, 1989. Other vectors and methods suitable for adaptation to the expression of polypeptides are well known in the art and are readily adapted to the specific circumstances.

By using the teachings provided herein and the knowledge in the art, a person skilled in the art will recognize that the efficacy of a particular expression system can be tested by transfecting packaging cells with a vector comprising a polynucleotide sequence encoding a reporter protein and measuring the expression using a suitable technique, for example, measuring fluorescence from a green fluorescent protein conjugate. Other suitable reporter genes are well known in the art.

A recombinant expression vector that comprises a polynucleotide sequence that encodes an immunogen may be used for production of the immunogen. Recombinant expression vectors include at least one regulatory expression sequence, such as a promoter or enhancer, that is operatively linked to the polynucleotide encoding the immunogen. Each of the expression vectors may be used to transform, transducer, or transfect an appropriate host cell for recombinant production of a respective immunogen. Suitable host cells for production of the immunogen include prokaryotes, yeast and higher eukaryotic cells (e.g., CHO and COS). The immunogen may each be isolated from the respective host cell or host cell culture using any one of a variety of isolation methods (e.g., filtration, diafiltration, chromatography (including affinity chromatography, high pressure liquid chromatography), and preparative electrophoresis) known and routinely practiced in the protein art. In certain embodiments, as described herein, the isolated immunogen may then be formulated with a pharmaceutically suitable excipient to provide an immunogenic composition.

Particular methods for producing polypeptides recombinantly are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). DNA sequencing can be performed as described in Sanger et al. (Proc. Natl. Acad. Sci. USA 74:5463 (1977)) and the Amersham International plc sequencing handbook and including improvements thereto.

### Vector Particles

In another embodiment, vector particles are provided. A vector particle comprises any one of the recombinant expression vectors described herein that comprise a polynucleotide sequence encoding at least one immunogen. In certain other embodiments, a vector particle comprises a recombinant expression system that comprises one recombinant expression vector (also called a first recombinant expression vector) comprising a polynucleotide sequence encoding at least one immunogen that induces a specific immune response. Also provided herein are methods for delivering a polynucleotide encoding at least one immunogen (as described herein) to a target cell. In particular embodiments, the target cell is an immune cell that is an antigen-presenting cell; in more specific embodiments and as described herein, the target cell is a dendritic cell. Such methods comprise contacting (i.e., permitting interaction) of the target cell with a vehicle that delivers the polynucleotide. As described herein, the recombinant expression vector may be multicistronic, encoding and directing expression of at least two immunogens. In particular embodiments, described in detail herein, methods for delivering the polynucleotide comprise contacting the cell by administering to a subject a vector particle that comprises a recombinant expression vector that contains a polynucleotide sequence that encodes the immunogen. The vector particles, recombinant expression vectors, polynucleotides, and immunogens are discussed in greater detail herein.

In certain embodiments, the vector particle is a viral vector particle and in other certain embodiments, the vector particle is a particle derived from a bacteria such as, for example, Listeria monocytogenes, Salmonella spp., Mycobacterium bovis, Escherichia coli, Shigella spp., and Yersinia spp. (see, e.g., Paterson, Semin. Immunol (2010) 22:183; Loessner, Expert Opin. Biol. Ther. (2004) 4:157; Daudel, Expert Rev. Vaccines (2007) 6:97). Exemplary viral vector particles include a lentiviral vector particle that comprises a lentiviral vector genome; a poxvirus vector particle that comprises a poxvirus vector genome; a vaccinia virus vector particle that comprises a vaccinia virus vector genome; an adenovirus vector particle that comprises a adenovirus vector genome; an adenovirus-associated virus vector particle that comprises a adenovirus-associated virus vector genome; a herpes virus vector particle that comprises a herpes virus vector genome (e.g., Herpes simplex virus I or II); or an alpha virus vector particle that comprises an alpha virus vector genome.

In a more particular embodiment, the vector particle is a lentiviral vector particle that comprises a lentiviral vector genome (which is described in detail above). Methods and compositions are provided herein for targeting cells and targeting dendritic cells (DCs) in particular by using a lentiviral vector particle (which may also be called a virion, a lentivirus particle) for delivering a sequence that encodes at least one immunogen to DCs. The lentiviral vector particle comprises an envelope glycoprotein variant derived from Sindbis virus E2, and a recombinant expression construct that comprises the genome that includes the sequences of interest, and optionally other components. The glycoprotein variant exhibits reduced binding to heparan sulfate compared to the glycoprotein from HR, a reference Sindbis virus strain. The envelope glycoprotein facilitates infection of dendritic cells by the lentiviral vector particles. “Facilitates” infection, as used herein, is the same as facilitates transduction and refers to the role of the envelope glycoprotein, acting alone or in concert with other molecules, in promoting or enhancing receptor-mediated entry of a pseudotyped retrovirus or lentivirus particle into a target cell.

In general, the lentiviral vector particles are produced by a cell line that contains one or more plasmid vectors and/or integrated elements that together encode the components necessary to generate functional vector particles. These lentiviral vector particles are typically not replication-competent, i.e., they are only capable of a single round of infection. Most often, multiple plasmid vectors or individual expression cassettes integrated stably into the producer cell chromosome are utilized to separate the various genetic components that generate the lentiviral vector particles; however, a single plasmid vector having all of the lentiviral components can be used. In one exemplification, the packaging cell line is transfected with one or more plasmids containing the viral vector genome, including LTRs, a cis-acting packaging sequence, and the sequences of interest (i.e., at least a nucleotide sequence encoding one immunogen), at least one plasmid encoding the virus enzymatic and structural components (e.g., gag and pol), and at least one plasmid encoding an Arbovirus envelope glycoprotein. Viral particles bud through the cell membrane and comprise a core that includes typically two RNA genomes containing the sequences of interest and an Arbovirus envelope glycoprotein that targets dendritic cells. In certain embodiments, the Arbovirus glycoprotein is a Sindbis virus E2 glycoprotein, and the glycoprotein is engineered to have reduced binding to heparan sulfate compared to E2 from the reference strain HR. This usually involves at least one amino acid change compared to the HR E2 glycoprotein sequence. As well, the E2 glycoprotein may be engineered to increase targeting specificity to dendritic cells.

Without wishing to be bound by theory, binding of the viral particle to a cell surface is believed to induce endocytosis, bringing the virus into an endosome, triggering membrane fusion, and allowing the virus core to enter the cytosol. For certain embodiments, which utilize integrating lentiviral vector particles, following reverse transcription and migration of the product to the nucleus, the genome of the virus integrates into the target cell genome, incorporating the sequences of interest into the genome of the target cell. To reduce the chance of insertional mutagenesis and to promote transient expression of a designated immunogen(s), however, other embodiments utilize non-integrating lentiviral vector particles (i.e., those which do not integrate into the target cell genome), but instead express the sequences of interest from an episome. In either instance, the infected DC then expresses the sequences of interest (e.g., an immunogen and optionally a stimulatory molecule). The immunogen can then be processed by dendritic cells and presented to T and B cells, generating an antigen-specific immune response. The specific pathway described above is not required so long as the dendritic cell is able to stimulate an antigen-specific immune response.

The viral particles can be administered to a subject in the immunogenic composition described herein to provide a prophylactic or therapeutic effect. Following infection of dendritic cells and expression of the immunogen product, an immune response is generated to the products.

Dendritic cells (DCs) are essential antigen presenting cells for the initiation and control of immune responses. DCs can develop along two pathways: one pathway is independent of monocytes and the second pathway is derived from monocytes (Mo-DCs). Blood monocytes, upon culture with GM-CSF and IL-4 acquire a dendritic morphology and strong capacities to initiate adaptive immunity (see, e.g., Bender et al., J. Immunol. Methods 196(2):121 (1996); Sallusto et al., J. Exp. Med. 179(4), 1109 (1994), including in vivo in humans (see, e.g., Dhodapkar, et al., J. Clin. Invest 104(2), 173 (1999); Schuler-Thurner, et al., J. Immunol. 165(6):3492 (2000)). A more effective immunogen-specific T cell responses may be achieved by using a vector particle vaccine, in particular a lentiviral vector particle system that efficiently delivers immunogens directly to Mo-DCs in vivo, without the need for ex vivo cellular manipulation. Human Mo-DCs express high levels of two C-type lectin receptors, mannose receptor (MMR) and DC-specific intercellular adhesion molecule-3-grabbing non-integrin (DC-SIGN). As described in greater detail herein, expression of immunogens may be targeted to Mo-DCs using a recombinant lentiviral vector engineered to target DC-SIGN.

A DC-SIGN-targeting envelope, SVGmu, consisting of an engineered Sindbis virus (SIN) glycoprotein that selectively binds DC-SIGN has been modified as described (see description herein and U.S. patent application Ser. No. 12/842,609; International Patent Application Publication No. WO 2011/011584). The lentiviral vector induced highly functional CD8 T cell immune responses after a single immunization in mice (see, e.g., Dai, et al., Proc. Natl. Acad. Sci. U.S.A (2009); Yang, et al., Nat. Biotechnol. 26(3), 326 (2008)). This prototype has been significantly advanced by two major modifications. The lentiviral vector described herein comprises a glycoprotein envelope (termed SINvar1) based on native SIN, an arbovirus known to infect dermal DCs via the DC-SIGN receptor (see, e.g., Gardner, et al., J. Virol. 74(24), 11849 (2000); Klimstra, et al., J. Virol. 77(22), 12022 (2003)) that is modified to prevent binding to ubiquitous heparan sulfate receptors (see, e.g., Klimstra et al., J. Virol. 72(9), 7357 (1998)). The SINvar1 envelope confers both increased productivity and in vivo function compared with the parental SVGmu envelope. The vector is also redundantly integration incompetent through the combination of a mutant Integrase (pol^{D64V}), rendering it non-functional (see, e.g., Apolonia, et al., Mol. Ther. 15(11), 1947 (2007)), and a vector backbone deleted of the U3 region of the LTR (up to att) and the 3′ LTR poly-purine tract (PPT). Thus, in addition to a disabled Integrase, the composition of the vector backbone prevents transcription of the full-length vector genome (self-inactivating mutation) resulting in single-LTR reverse transcribed episomal dsDNA circles in the infected DC, which are not a template for chromosomal integration (see, e.g., Bayer, et al., Mol. Ther. 16(12):1968 (2008); Breckpot et al., J. Virol. (2010); Ma et al., Mol. Ther. 10(1):139 (2004)). Approximately 75% of the parental HIV genome has been removed from DC-NILV, including all of the regulatory and accessory proteins except for Rev. After a single injection, DC-NILV induces highly robust tumor antigen-specific CD8 T cell response. The potency of lentivector vaccination is dependent at least in part on engagement of TLR3 and TLR7 pattern recognition receptors (see, e.g., Beignon et al., J. Virol. (2009); Breckpot et al., supra).

In additional embodiments, the lentiviral vector particles may be produced as described in U.S. Pat. No. 8,323,662. In particular, the lentiviral vector may be produced using a packaging system such that the envelope glycoprotein is highly mannosylated. One aspect of the disclosure provides a method of generating a pseudotyped lentiviral vector particle comprising: (a) culturing in a culture medium comprising a mannosidase inhibitor, preferably a mannosidase I inhibitor, and a virus packaging cell comprising: (1) a lentiviral vector genome comprising a polynucleotide encoding an exogenous antigen, (2) a polynucleotide encoding an alphavirus glycoprotein that preferentially binds cells expressing DC-SIGN, and (3) a polynucleotide encoding a SAMHD1 inhibitor such as Vpx; and (b) isolating a pseudotyped lentiviral vector particle that preferentially binds cells expressing DC-SIGN. In some or any of the embodiments described herein, the Vpx protein comprises an amino acid sequence that is at least 80% identical to SIVmac Vpx.

Viral Vector Envelope

Arthropod-borne viruses (Arboviruses) are viruses that are transmitted to a host, such as humans, horses, or birds by an infected arthropod vector such as a mosquito. Arboviruses are further divided into sub-families of viruses including alphaviruses and flaviviruses, which have a single-stranded RNA genome of positive polarity and a glycoprotein-containing envelope. For example, dengue fever virus, yellow fever virus and West Nile virus belong to the flavivirus family, and Sindbis virus, Semliki Forest virus and Venezuelan Equine Encephalitis virus, are members of the alphavirus family (see, e.g., Wang et al., J. Virol. 66, 4992 (1992)). The envelope of Sindbis virus includes two transmembrane glycoproteins (see, e.g., Mukhopadhyay et al. Nature Rev. Microbiol. 3, 13 (2005)): E1, believed to be responsible for fusion, and E2, believed to be responsible for cell binding. Sindbis virus envelope glycoproteins are known to pseudotype other retroviruses, including oncoretroviruses and lentiviruses.

As discussed herein, an arbovirus envelope glycoprotein can be used to pseudotype a lentiviral-based vector genome. A “pseudotyped” lentivirus is a lentiviral particle having one or more envelope glycoproteins that are encoded by a virus that is distinct from the lentiviral genome. The envelope glycoprotein may be modified, mutated or engineered as described herein.

The envelope of Sindbis virus and other alphaviruses incorporates into the lipid bilayer of the viral particle membrane, and typically includes multiple copies of two glycoproteins, E1 and E2. Each glycoprotein has membrane-spanning regions; E2 has an about 33 residue cytoplasmic domain whereas the cytoplasmic tail of E1 is very short (about 2 residues). Both E1 and E2 have palmitic acids attached in or near the membrane-spanning regions. E2 is initially synthesized as a precursor protein that is cleaved by furin or other Ca2+-dependent serine proteinase into E2 and a small glycoprotein called E3. Located between sequences encoding E2 and E1 is a sequence encoding a protein called 6K. E3 and 6K are signal sequences which serve to translocate the E2 and E1 glycoproteins, respectively, into the membrane. In the Sindbis virus genome, the coding region for Sindbis envelope proteins includes sequence encoding E3, E2, 6K, and E1. As used herein, “envelope” of an arbovirus virus includes at least E2, and may also include E1, 6K, and E3. Exemplary envelope glycoproteins of Sindbis virus, strain HR, is disclosed in US20120328655. In certain particular alternative embodiments, an E3/E2 glycoprotein, wherein the E3 sequence corresponds to residues 1-65 of SEQ ID NO:20 as disclosed in US20120328655, or a variant thereof and wherein residues 62-65 are RSKR, may be incorporated into a pseudotyped viral envelope. Sequences of envelope glycoproteins for other arboviruses can be found in publically available databases, such as GenBank. For example, sequences encoding Dengue virus glycoproteins can be found in Accession GQ252677.1 (among others in GenBank) and in the virus variation database at NCBI (GenBank accessions and virus variation database are incorporated by reference for envelope glycoprotein sequences) and an exemplary sequence encoding Venezuelan equine encephalitis virus envelope glycoproteins in Accession NP_040824.1 (incorporated by reference for sequences of envelope glycoproteins).

Although the cellular receptor(s) on dendritic cells for alphaviruses, and Sindbis virus in particular, have not been definitively identified to date, one receptor appears to be DC-SIGN (see, e.g., Klimstra et al., J. Virol. 77:12022, 2003). The use of the terms “attachment,” “binding,” “targeting” and the like are used interchangeably and are not meant to indicate a mechanism of the interaction between Sindbis virus envelope glycoprotein and a cellular component. DC-SIGN (Dendritic Cell Specific ICAM-3 (Intracellular Adhesion Molecules 3)-Grabbing Nonintegrin; also known as CD209) is a C-type lectin-like receptor capable of rapid binding and endocytosis of materials (see, e.g., Geijtenbeek et al. Annu. Rev. Immunol. 22: 33-54, 2004). E2 appears to target virus to dendritic cells through DC-SIGN. As shown herein, cells expressing DC-SIGN are transduced by viral vector particles pseudotyped with Sindbis virus E2 better (at least 2-fold, at least 3-fold, at least 4-fold, at least 5-fold, at least 6-fold, at least 7-fold, at least 8-fold, at least 9-fold, or at least 10-fold better) than isogenic cells that do not express DC-SIGN. The mechanism of how E2 glycoprotein facilitates viral infection appears to involve DC-SIGN, possibly through direct binding to DC-SIGN or causing a change in conformation or some other mechanism. Regardless of the actual mechanism, the targeting by E2 is preferential for cells expressing DC-SIGN, namely dendritic cells.

Sindbis virus also appears to bind to cells via heparan sulfate (see, e.g., Klimstra et al., J. Virol. 72: 7357, 1998; Burmes et al., J. Virol. 72: 7349, 1998). Because heparan sulfate and other cell surface glycosaminoglycans are found on the surface of most cell types, it is desirable to reduce interaction between heparan sulfate and Sindbis envelope glycoproteins. This can be accomplished by diminishing the binding of Sindbis virus envelope to heparan sulfate or increasing the binding, e.g., increasing avidity, of Sindbis virus envelope to dendritic cells or both. As a result, nonspecific binding to other molecules, which may be expressed by other cell types and which may occur even if the envelope is specific for DC-SIGN, is reduced, and the improved specificity may serve to avoid undesired side effects, such as side effects that may reduce the desired immune response, or side effects associated with off-target transduction of other cell types. Alternatively or in addition to the advantages of relatively specific transduction of cells expressing DC-SIGN, viral particles pseudo-typed with Sindbis virus envelope E2 glycoprotein may offer other advantages over viral particles pseudo-typed with glycoproteins such as VSV-G. Examples of such advantages include reduced complement-mediated lysis and/or reduced neuronal cell targeting, both of which are believed to associate with administration of VSV-G pseudo-typed viral particles.

In various exemplifications, the lentiviral vector particles specifically bind to cells expressing DC-SIGN and have reduced or abrogated binding to heparan sulfate. That is, a Sindbis virus envelope E2 glycoprotein may be modified to preferentially direct the virus to dendritic cells that express DC-SIGN relative to other cell types. Based on information obtained from structural studies and molecular modeling among other studies, variant sequences of envelope proteins, especially E2 and E1 glycoproteins, are designed and generated such that the glycoproteins maintain their functions as envelope proteins, but have the desired binding specificity, avidity, or level of binding. Candidate variant sequences may be created for each glycoprotein and assayed using the methods described below, or other methods known in the art, to identify envelope glycoproteins with the most desirable characteristics.

Certain variant sequences of Sindbis E2 have at least one amino acid alteration at residue 160 as compared to SEQ ID NO:1. Residue 160 is deleted or changed to an amino acid other than glutamic acid. An alteration is most commonly a substitution of at least one amino acid, but alternatively can be an addition or deletion of one or more amino acids. Preferably, any additional amino acids are few in number and do not comprise an antigenic epitope (e.g., hemagglutinin tag sequence), which may compromise safety. When there are two or more alterations, they can both be of the same type (e.g., substitution) or differing types (e.g., a substitution and a deletion). Multiple alterations can be scattered or located contiguously in the protein sequence.

By way of example, variant sequences comprise at least one amino acid alteration in the region of about residue 50 to about residue 180 of SEQ ID NO:1. Within this region are amino acids that are involved with binding to heparan sulfate. By reducing the net positive charge of E2, electrostatic interaction with heparan sulfate can be reduced, resulting in decreased binding to heparan sulfate. Candidate positively charged amino acids in this region include lysines at residues 63, 70, 76, 84, 97, 104, 129, 131, 133, 139, 148, 149, 159 and arginine at residues 65, 92, 128, 137, 157, 170, 172 (see, e.g., Bear et al., Virology 347: 183-190, 2006) (see SEQ ID NO:1). At least several of these amino acids are directly implicated in E2 binding to heparan sulfate. Net positive charge can be reduced by deletion of lysine or arginine or substitution of lysine or arginine with a neutral or negatively charged amino acid. For example, one or more of these lysines and arginines may be replaced with glutamic or aspartic acid. Certain embodiments have at least one substitution of lysine 70, 76 or 159. Exemplary amino acid sequences of the E2 glycoprotein are set forth in SEQ ID NOS:3-16. In cases where E2 is expressed as a polyprotein with E3, the lysine located adjacent to the natural E3/E2 cleavage site is maintained—that is, the recognition sequence and cleavage site is unaltered. Alternatively, the native endopeptidase cleavage site sequence is replaced with a recognition sequence for a different endopeptidase.

Certain variants of E2 are also modified in a way that positively impacts binding to dendritic cells. Alteration of the glutamic acid found at residue 160 in the reference HR sequence can improve binding to dendritic cells (see, e.g., Gardner et al., J. Virol. 74, 11849, 2000). Alterations, such as a deletion of residue 160 or substitution of residue 160 are found in certain variants. In particular variants, a non-charged amino acid is substituted for Glu, in other variants, a non-acidic amino acid is substituted for Glu. Typically, Glu160 is replaced with one of the small or aliphatic amino acids, including glycine, alanine, valine, leucine or isoleucine.

Other variants comprise two or more amino acid alterations. Typically in these variants one of the alterations is Glu160 and the remaining alteration(s) are changes of one or more of the lysines and arginines in the region spanning residue about 50 to about 180 of SEQ ID NO:1. Certain of the variants comprise an alteration of Glu160 to a non-acidic residue or deletion and one or more alterations of lysine 70, lysine 76, or lysine 159 with a non-basic amino acid. Some specific variants comprise a Glu160 to Gly, Lys 70 to Glu, and Lys 159 to Glu; a Glu 160 to Gly, Lys 70, 76 and 159 to Glu; a deletion of Glu 160 and Lys 70 and 159 to Glu; and a deletion of Glu 160 and Lys 70, 76, and 159 to Glu. (See, e.g., SEQ ID NOS:3-16.)

In certain embodiments, E2 protein is first expressed as a polyprotein in fusion with at least E3 or in fusion with a leader sequence. Regardless of whether the leader sequence is E3 or another sequence, E2 in the viral envelope should be free of the E3 or other leader sequence. In other words, E2 is preferably not an E3/E2 fusion protein (e.g., the E3/E2 fusion protein called SVGmu). In certain embodiments, E2 is expressed as part of E3-E2-6K-E1 polyprotein. Sindbis virus naturally expresses E2 as part of a polyprotein and the junction regions for E3/E2, E2/6K, and 6K/E1 have sequences recognized and cleaved by endopeptidases. Normally, the E3/E2 junction is cleaved by furin or a furin-like serine endopeptidase between residues 65 and 66. Furin has specificity for paired arginine residues that are separated by two amino acids. To maintain E3/E2 cleavage by furin, residues 62-66 (RSKRS; SEQ ID NO: 26) should maintain the two arginine residues with two amino acid separation and the serine residue. Alternatively, a different cleavage sequence can be used in place of the E3/E2 furin cleavage sequence or any of the other cleavage sequences. Recognition and cleavage sites can be incorporated for endopeptidases, including, without limitation, aspartic endopeptidases (e.g., cathepsin D, chymosin, HIV protease), cysteine endopeptidases (bromelains, papain, calpain), metalloendopeptidases, (e.g., collagenase, thermolysin), serine endopeptidases (e.g., chymotrypsin, factor IXa, factor X, thrombin, trypsin), streptokinases. The recognition and cleavage site sequences for these enzymes are well known.

Amino acids in E2, other than those already mentioned, may also be altered. Generally, a variant E2 sequence will have at least 80% sequence amino acid identity to the reference E2 sequence, or it may have at least 82%, at least 85%, at least 87%, at least 90%, at least 92%, at least 95%, or at least 98% sequence identity. The variant glycoprotein should exhibit biological function, such as the ability to facilitate infection of dendritic cells by a viral particle having an envelope comprising E2. Experiments have identified regions of envelope glycoproteins that appear to have an important role in various aspects of viral assembly, attachment to cell surface, and infection. When making variants, the following information can be used as guidelines. The cytoplasmic tail of E2—approximately residues 408 to 415—is important for virus assembly (see, e.g., West et al. J. Virol. 80: 4458-4468, 2006; incorporated in its entirety). Other regions are involved in forming secondary structure (approximately residues 33-53), and involved in transport and protein stability (approximately residues 86-119) (see, e.g., Navaratmarajah et al., J. Virol. 363:124-147, 2007; incorporated in its entirety). The variant may retain hydrophobic character of a region that spans the membrane, approximately residues 370-380. The variant may retain one or both N-linked glycosylation sites residues NIT (residues 196-198) and NFT (residues 318-320) and may retain one or more of the sites that are palmitoylated (C-396, C416 and C417) (see, e.g., Strauss et al., Microbiol. Rev. 58, 491-562, 1994; pp. 499-509 incorporated herein by reference in its entirety). On the other hand, many regions of E2 may be altered without deleterious event. For example, insertions of transposons at many different locations in E2 still resulted in viable virus (see, e.g., Navaratmarajah, supra).

In certain embodiments, a tag peptide may be incorporated into E3, 6K, or E1 proteins. For some purposes, a tag may be incorporated into E2, but a tag is not desirable for use in a product for administration to human patients. A tag peptide, which is a short sequence (e.g., 5-30 amino acids), can be used to facilitate detection of envelope expression and its presence in viral particles. For detection purposes, a tag sequence will typically be detectable by antibodies or chemicals. Another use for a tag is to facilitate purification of viral particles. A substrate containing a binding partner for the tag can be used to absorb virus. Elution of the virus can be accomplished by treatment with a moiety that displaces the tag from the binding partner or when the tag sequence is in linkage with a cleavable sequence, treatment with the appropriate endopeptidase will conveniently allow release of virus. (See, for example, QiaGEN® catalog, Factor Xa Protease System). Removal of the tag peptide is generally desirable for safety purposes of the virus particles use in animal subjects. If the tag is not removed, an immune response to the tag may occur.

Suitable tags include, without limitation, FLAG (DYKDDDDK) (SEQ ID NO:35) (U.S. Pat. No. 4,703,004, incorporated in its entirety), for which antibodies are commercially available, chitin binding protein, maltose binding protein, glutathione-S-transferase, poly(His) (U.S. Pat. No. 4,569,794, incorporated in its entirety), thioredoxin, HA (hemagglutinin)-tag, among others. Poly(His) can be adsorbed onto affinity media containing bound metal ions, such as, nickel or cobalt, and eluted with a low pH medium.

The vector particles may be evaluated to determine the specificity of the envelope glycoprotein incorporated into the virus that targets dendritic cells. For example, a mixed population of bone marrow cells can be obtained from a subject and cultured in vitro. Alternatively, isogenic cells lines that express or do not express DC-SIGN can be obtained and used. The recombinant virus can be administered to the mixed population of bone marrow cells or isogenic cell lines, and expression of a reporter gene incorporated into the virus can be assayed in the cultured cells. Certain embodiments may employ a limiting dilution analysis, in which the mixed population of cells is split into separate parts, which are then separately incubated with decreasing amounts of virus (e.g., 2-fold, 5-fold, 10-fold less virus in each part). In some embodiments, at least about 50%, or at least about 60%, 70%, 80% or 90%, or at least about 95% of infected cells in the mixed cell population are dendritic cells that express DC-SIGN. In certain embodiments, the ratio of infected dendritic cells to infected non-dendritic cells (or non DC-SIGN expressing cells) is at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 40:1, at least about 50:1, at least about 100:1, at least about 200:1, at least about 500:1, at least about 1000:1, at least about 5000:1, at least about 10,000:1, or more. For limiting dilution, greater selectivity is typically seen at higher dilutions (i.e., lower amounts) of input virus.

Activity of pseudotyped viral particles can be determined by any of a variety of techniques. For example, a preferred method to measure infectivity efficiency (IU, infectious units) is by administering viral particles to cells and measuring expression of a product encoded in the vector genome. Any product that can be assayed may be used. One convenient type of product is a fluorescent protein, such as green fluorescent protein (GFP). Other products that can be used include proteins expressed on a cell surface (e.g., detection by antibody binding), enzymes, and the like. If the product is an antigen and cells are dendritic cells, infectivity/activity can be assessed by determining an immune response. Furthermore, it is possible to ascertain side effects in a mammal. The ability to specifically target dendritic cells can also be tested directly, for example, in cell culture as described below.

Vector particles, which include the viral particles described herein can also be prepared and tested for their selectivity and/or their ability to facilitate penetration of the target cell membrane. Viral particles that have an envelope with unmodified glycoproteins can be used as controls for comparison. Briefly, cells expressing a receptor for an envelope glycoprotein are infected by the virus using a standard infection assay. After a specified time, for example 48 hours post-infection, cells can be collected and the percentage of cells infected by the virus can be determined by flow cytometry, for example. Selectivity can be scored by calculating the percentage of cells infected by virus. Similarly, the effect of a variant envelope glycoprotein on viral titer can be quantified by dividing the percentage of cells infected by virus comprising a variant envelope by the percentage of cells infected by virus comprising the corresponding wild type (unmodified) envelope glycoprotein. A particularly suitable variant will have the best combination of selectivity and infectious titer. Once a variant is selected, viral concentration assays may be performed to confirm that these viruses can be concentrated without compromising activity. Viral supernatants are collected and concentrated by ultracentrifugation. The titers of viruses can be determined by limited dilution of viral stock solution and infection of cells expressing the receptor for the envelope glycoprotein, measuring the expression of a product expressed by the viruses as described above.

The entry of a lentiviral vector particle into a target cell is another type of evaluation of activity. BlaM-Vpr (beta-lactamase Vpr) fusion protein has been used to evaluate HIV-1 viral penetration; a fusion of BlaM and a Sindbis virus envelope glycoprotein, such as E1 or an E2/E1 fusion protein can be used to assess the efficacy of an envelope protein in facilitating fusion and penetration into a target cell. Viral particles may be prepared, for example, by transient transfection of packaging cells with one or more vectors comprising the viral elements, BlaM-Vpr, and the variant envelope of interest (and an affinity molecule if appropriate). The resulting viruses can be used to infect cells expressing a molecule the targeting molecule (or affinity molecule) specifically binds in the absence or presence of the free inhibitor of binding (such as an antibody). Cells can then be washed with CO₂-independent medium and loaded with CCF2 dye (Aurora Biosciences, San Diego, Calif.). After incubation at room temperature to allow completion of the cleavage reaction, the cells can be fixed by paraformaldehyde and analyzed by flow cytometry and microscopy. The presence of blue cells indicates the penetration of viruses into the cytoplasm; fewer blue cells would be expected when blocking antibody is added (see, e.g., Cavrois et al., Nat. Biotechnol. 20:1151-54, 2002).

To investigate whether penetration is dependent upon a low pH, and to identify envelope glycoproteins with the desired pH dependence, NH₄Cl or other compound that alters pH can be added at the infection step (NH₄Cl will neutralize the acidic compartments of endosomes). In the case of NH₄Cl, the disappearance of blue cells will indicate that penetration of viruses is low pH-dependent. In addition, to confirm that the activity is pH-dependent, lysosomotropic agents, such as ammonium chloride, chloroquine, concanamycin, bafilomycin A1, monensin, nigericin, etc., may be added into the incubation buffer. These agents elevate the pH within the endosomal compartments (see, e.g., Drose et al., J. Exp. Biol. 200, 1-8, 1997). The inhibitory effect of these agents will reveal the role of pH for viral fusion and entry. The different entry kinetics between viruses displaying different fusogenic molecules may be compared and the most suitable selected for a particular application.

PCR-based entry assays can be utilized to monitor reverse transcription and measure kinetics of viral DNA synthesis as an indication of the kinetics of viral entry. For example, viral particles comprising a particular envelope protein molecule are incubated with target cells, such as 293T cells, DCs, or any other cells that have been engineered to express, or which naturally express, the appropriate binding partner (receptor) for the envelope protein molecule. Either immediately, or after a time increment (to allow infection to occur), unbound viruses are removed and aliquots of the cells are analyzed for viral nucleic acids. DNA is extracted from these aliquots and subjected to amplification analysis, generally in a semi-quantitative assay, primed with LTR-specific primers. The appearance of LTR-specific DNA products indicates the success of viral entry.

Following viral infection with the viral vector particle, the immunogen is expressed by the target dendritic cells. If contacted ex vivo, the target dendritic cells are then transferred back to the patient, for example by injection, where they interact with immune cells that are capable of generating an immune response against the desired antigen. In preferred embodiments, the recombinant virus is injected into the patient where it transduces the targeted dendritic cells in situ. The dendritic cells then express the particular antigen associated with a disease or disorder to be treated, and the patient is able to mount an effective immune response against the disease or disorder.

The viral vector genome may contain a polynucleotide sequence encoding more than one immunogen, and upon transduction of a target dendritic cell, generates immune responses to each immunogen delivered to the cell. In some embodiments, the immunogens are related to a single disease or disorder. In other embodiments, the immunogens are related to multiple diseases or disorders.

In some of the vector particles, DC maturation factors that activate and/or stimulate maturation of the DCs are delivered in conjunction with the immunogen-encoding sequence of interest. In certain alternative embodiments, the DCs are activated by delivery of DC maturation factors prior to, simultaneously with, or after delivery of the vector particles. DC maturation factors may be provided separately from administration of the vector particles.

As described herein, one or more immune modulation or DC maturation factors can be encoded by one or more sequences that are contained in the vector particle and expressed after the particle enters or infects a dendritic cell. The sequences encoding immune modulation factors can also be provided in a separate vector that is co-transfected with the vector particle encoding one or more immunogens in a packaging cell line.

The methods described herein may be used for adoptive immunotherapy in a subject. As described above, an immunogen against which an immune response is desired is identified. A polynucleotide encoding the desired immunogen(s) is obtained and packaged into a vector particle. Target dendritic cells are obtained from the patient and transduced with the vector particle containing a polynucleotide that encodes the desired immunogen. The dendritic cells are then transferred back into the patient.

The vector particles (e.g., the viral vector particles described herein) may be injected in vivo, where the particles infect DCs and deliver the immunogen-encoding nucleotide sequence of interest. The amount of viral particles is at least 3×10⁶ infectious units (IU), and can be at least 1×10⁷ IU, at least 3×10⁷ IU, at least 1×10⁸ IU, at least 3×10⁸ IU, at least 1×10⁹ IU, or at least 3×10⁹ IU. At selected intervals, DCs from the recipient's lymphoid organs may be used to measure expression, for example, by observing marker expression, such as GFP or luciferase if co-expressed by a polynucleotide sequence present in the recombinant expression vector included in the vector particle. Nucleic acid monitoring techniques and measurements of reverse transcriptase (RT) activity can also be used to analyze the biodistribution of vector particles when the vector particle is a lentiviral vector particle. T cells from peripheral blood mononuclear cells, lymph nodes, spleens, or malignant or target pathogen-infected tissue of vector particle (including lentiviral vector particle) treated recipients may be measured from the magnitude and durability of response to antigen stimulation. Tissue cells other than DCs, such as epithelial cells and lymphoid cells, may be analyzed for the specificity of in vivo gene delivery.

### Immune Response

As described herein, methods are provided for inducing an immune response to an immunogen. Cells of the immune system that are involved in an immune response are referred to, generally, as immune cells and include a lymphocyte and a non-lymphoid cell such as accessory cell. Lymphocytes are cells that specifically recognize and respond to foreign antigens, and accessory cells are those that are not specific for certain antigens but are involved in the cognitive and activation phases of immune responses. For example, mononuclear phagocytes (macrophages), other leukocytes (e.g., granulocytes, including neutrophils, eosinophils, basophils), and dendritic cells function as accessory cells in the induction of an immune response. The activation of lymphocytes by a foreign antigen leads to induction or elicitation of numerous effector mechanisms that function to eliminate the antigen. Accessory cells such as mononuclear phagocytes that affect or are involved with the effector mechanisms are also called effector cells.

Major classes of lymphocytes include B lymphocytes (B cells), T lymphocytes (T cells), and natural killer (NK) cells, which are large granular lymphocytes. B cells are capable of producing antibodies. T lymphocytes are further subdivided into helper T cells (CD4+ (also referred to herein and in the art as CD4)) and cytolytic or cytotoxic T cells (CD8+ (also referred to herein and in the art as CD8)). Helper cells secrete cytokines that promote proliferation and differentiation of the T cells and other cells, including B cells and macrophages, and recruit and activate inflammatory leukocytes. Another subgroup of T cells, called regulatory T cells or suppressor T cells actively suppress activation of the immune system and prevent pathological self-reactivity, that is, autoimmune disease.

The methods described herein for inducing an immune response are useful for inducing a cell-mediated immune response involving various types of T cells (i.e., T lymphocytes). In a cell mediated response, the various types of T lymphocytes act to eliminate an antigen by a number of mechanisms. For example, helper T cells that are capable of recognizing specific antigens may respond by releasing soluble mediators such as cytokines to recruit additional cells of the immune system to participate in an immune response. Also, cytotoxic T cells are capable of specifically recognizing an antigen and may respond by binding to and destroying or damaging an antigen-bearing cell or particle. The methods described herein for inducing an immune response may also induce a humoral response, also called a B cell response herein and in the art. A humoral response includes production of antibodies that specifically bind to an antigen (or immunogen). Antibodies are produced by differentiated B lymphocytes known as plasma cells.

Whether an immune response is induced and the type of immune response induced in a host or subject may be determined by any number of well-known immunological methods described herein and with which those having ordinary skill in the art will be familiar. As described herein, methods and techniques for determining the presence and level of an immune response include, for example, fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, immunoassays, (such as enzyme-linked immunosorbant assays (ELISA), radioimmunoassay, immunoblotting, immunohistochemistry, and the like), surface plasmon resonance, cell-based assays such as those that use reporter genes, and functional assays (e.g., assays that measure immune function and immunoresponsiveness).

Such assays include, but need not be limited to, in vivo or in vitro determination of the presence and level of soluble antibodies, soluble mediators such as cytokines (e.g., IFN-γ, IL-2, IL-4, IL-10, IL-12, IL-6, IL-23, TNF-α, and TGF-β), lymphokines, chemokines, hormones, growth factors, and the like, as well as other soluble small peptide, carbohydrate, nucleotide and/or lipid mediators Immunoassays also include determining cellular activation state changes by analyzing altered functional or structural properties of cells of the immune system, for example, cell proliferation, altered motility, induction of specialized activities such as specific gene expression or cytolytic behavior; cell maturation, such as maturation of dendritic cells in response to a stimulus; alteration in relationship between a Th1 response and a Th2 response; cellular differentiation by cells of the immune system, including altered surface antigen expression profiles or the onset of apoptosis (programmed cell death). Procedures for performing these and similar assays are may be found, for example, in Lefkovits (Immunology Methods Manual: The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

Determining the presence and/or level of antibodies that specifically bind to an immunogen and the respective designated antigen of interest may be determined using any one of several immunoassays routinely practiced in the art, including but not limited to, ELISAs, immunoprecipitation, immunoblotting, countercurrent immunoelectrophoresis, radioimmunoassays, dot blot assays, inhibition or competition assays, and the like (see, e.g., U.S. Pat. Nos. 4,376,110 and 4,486,530; Harlow et al., Antibodies: A Laboratory Manual, Cold Spring Harbor Laboratory (1988)) Immunoassays may also be performed to determine the class and isotype of an antibody that specifically binds to an immunogen. Antibodies (polyclonal and/or monoclonal or antigen-binding fragments thereof), which specifically bind to an immunogen and which may be used as controls in immunoassays detecting an antibody-specific immune response in an immunized subject, may generally be prepared by any of a variety of techniques known to persons having ordinary skill in the art. See, e.g., Harlow et al., Antibodies: A Laboratory Manual, Cold Spring Harbor Laboratory (1988); Peterson, ILAR J. 46:314-19 (2005); (Kohler et al., Nature, 256:495-97 (1976); Kohler et al., Eur. J. Immunol. 6:511-19 (1975); Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); Antibodies: A Laboratory Manual, Harlow and Lane (eds.), Cold Spring Harbor Laboratory Press (1988); see also, e.g., Brand et al., Planta Med. 70:986-92 (2004); Pasqualini et al., Proc. Natl. Acad. Sci. USA 101:257-59 (2004). The immunogen, or immunogenic fragments thereof, or a cell or particle bearing the immunogen or immunogenic fragment thereof may be used for immunizing an animal for production of either polyclonal antibodies or monoclonal antibodies.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry) Immune cell proliferation and clonal expansion resulting from an antigen-specific elicitation or stimulation of an immune response may be determined by isolating lymphocytes, such as spleen cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

The level of a CTL immune response and the level of a memory CD4 T cell response may be determined by any one of numerous immunological methods described herein and routinely practiced in the art. The level of a CTL immune response may be determined prior to administration of any one of the compositions, vectors, or vector particles described herein and then used for comparison with the level of CTL immune response at an appropriate time point after one or more administrations of the compositions, vectors, or vector particles that provide memory CD4 T cell help. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

As used herein, a binding partner or an antibody is said to be “immunospecific,” “specific for” or to “specifically bind” an immunogen of interest if the antibody reacts at a detectable level with the immunogen or immunogenic fragment thereof, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and an antibody specifically binds to the immunogen of interest if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of binding partners or antibodies can be readily determined using conventional techniques, for example, those described by Scatchard et al. (Ann. N.Y. Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to a binding partner (or ligand) in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993)).

A biological sample may be obtained from the subject for determining the presence and level of an immune response to an immunogen and/or the respective designated antigen in the subject who has received any one or more of the immunogenic compositions described herein, such as an immunogenic composition comprising an immunogen and an immunogenic composition comprising a recombinant expression vector comprising a nucleotide sequence encoding the immunogen or who has received both immunogenic compositions, including one or more compositions comprising an adjuvant according to the methods described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

With respect to all immunoassays and methods described herein for determining an immune response, a person skilled in the art will also readily appreciate and understand which controls are appropriately included when practicing these methods. Concentrations of reaction components, types of buffers, temperature, and time periods sufficient to permit interaction of the reaction components can be determined and/or adjusted according to methods described herein and with which persons skilled in the art are familiar.

### Methods of Inducing an Immune Response

Methods are provided herein that comprise administering at least two different immunogenic compositions for inducing an adaptive, antigen-specific immune response against one or more antigens. Dual immunization of a subject with the immunogenic compositions as described herein results in induction of a humoral immune response and a cellular immune response (including a CD4 T cell response and a CD8 T cell response). The two immunogenic compositions may be administered concurrently or sequentially in either order. Accordingly, provided herein are methods for inducing a humoral immune response and a cellular response, which comprises a CD4 T cell response and a CD8 T cell response (and which may include a cytotoxic T cell response), wherein each of the immune responses is specific for an immunogen(s) and thereby specific for the respective designated antigen(s). These methods comprise administering an immunogenic composition that comprises at least one immunogen, (which is isolated and/or recombinantly produced), and administering a second immunogenic composition that comprises a recombinant expression vector that encodes and directs expression of the immunogen.

In one embodiment, methods are provided for inducing an immune response specific for one or more designated antigens in a subject by administering an immunogenic composition that comprises at least one immunogen capable of eliciting a specific immune response against a designated antigen (which, for convenience, may be called herein a first designated antigen). The methods further comprise concurrently administering or sequentially administering (i.e., prior to or subsequent to) another (i.e., second, different) immunogenic composition that comprises a recombinant expression vector comprising a nucleotide sequence that encodes the immunogen. The recombinant expression vector further comprises at least one regulatory sequence operatively linked to the nucleotide sequence that encodes the immunogen, and, thus, the recombinant expression vector is capable of directing expression of the immunogen.

In certain embodiments, the recombinant expression vector administered according to these methods for inducing an immune response is incorporated into a vector particle (e.g., a virus vector particle or a cell particle). The recombinant expression vector or vector particle comprising the vector is constructed in a manner that enables the particle to be introduced into (i.e., delivered to) a target cell. In certain embodiments, the target cell is an antigen-presenting cell. In more specific embodiments, the target cell is a professional antigen-presenting cell such as a dendritic cell. The immunogen (or a fragment thereof) is then expressed in the target cell, and the immunogen or a fragment thereof is presented on the surface of the antigen-presenting cell and induces an immune response specific for the immunogen and thereby for the respective designated antigen.

The immunogenic composition that comprises at least one immunogen (first immunogenic composition) may further comprise at least one adjuvant that is pharmaceutically or physiologically suitable for administering to the subject in need thereof to whom the immunogenic compositions are administered. The immunogenic composition that comprises a recombinant expression vector (second immunogenic composition) may also further comprise an adjuvant. If both the first composition and the second composition comprise an adjuvant, the adjuvants may be the same or different. Immunogens, the respective designated antigens, adjuvants, and recombinant expression vectors and vector particles are discussed in detail herein.

In another embodiment, the immunogenic composition comprising the at least one immunogen (which may further comprise an adjuvant) is first administered followed by administration of the immunogenic composition that comprises the recombinant expression vector concurrently with administration of the immunogenic composition comprising the at least one immunogen (which may further comprise an adjuvant). In other words, the immunogenic composition comprising the at least one immunogen (which may further comprise an adjuvant) is the first or priming immunization and the immunogenic composition that comprises the recombinant expression vector and the second dose of the immunogenic composition comprising the at least one immunogen (which may further comprise an adjuvant) both are administered concurrently as boosting compositions.

In other embodiments, methods are provided for inducing an immune response, wherein the immunogenic composition that comprises at least one immunogen further comprises at least one additional immunogen (or at least a second immunogen). In other embodiments of the methods described herein, the recombinant expression vector that encodes the immunogen and that is included in the second immunogenic composition also encodes and directs expression of at least one additional immunogen. In still another embodiment, the immunogenic composition that comprises at least one immunogen further comprises at least one additional immunogen and the recombinant expression vector included in the other (or second) immunogenic composition encodes and directs expression of at least one additional immunogen. The immunogen included in each of the first and second immunogenic compositions may be the same or different. In particular embodiments, the at least one additional immunogen included in the first composition and encoded by the recombinant expression vector included in the second immunogenic composition are the same. As discussed in detail herein, when more than one immunogen is included in the immunogenic composition or encoded by the recombinant expression vector, each immunogen may induce a specific immune response for the same or different designated antigens.

Accordingly, in one specific embodiment, methods are provided wherein the immunogenic composition that comprises at least one immunogen (and which may further comprise an adjuvant) further comprises at least one additional immunogen (i.e., at least two, at least three, at least four, at least five, at least six or more immunogens which may be restated as two, three, four, five, six or more immunogens)). In certain embodiments, the immunogenic composition that comprises at least two immunogens (e.g., two, three, four, five, six or more immunogens) forms a multivalent immunogenic composition. In instances when the two or more immunogens are combined with an adjuvant, the immunogenic composition may comprise each immunogen formulated separately with an adjuvant and then the adjuvanated immunogens are combined to form the immunogenic composition that is administered to the subject. Alternatively, the two or more immunogens may be combined with an adjuvant and formulated together to form the immunogenic composition. In certain specific embodiments, one or more of each additional immunogen (e.g., the second, third, fourth, fifth, sixth immunogen, etc.) may induce an immune response to the same designated antigen as the first immunogen. In other specific embodiments, each additional immunogen (e.g., second, third, fourth, fifth immunogen, etc.) may induce an immune response specific for a different designated antigen (e.g., a second, third, fourth, fifth, sixth etc. designated antigen), respectively.

As described above, in certain embodiments, methods are provided wherein the recombinant expression vector in an immunogenic composition may be multicistronic and comprise a nucleotide sequence that encodes at least one additional immunogen (i.e., at least two, at least three, at least four, at least five, at least six, or more immunogens which may be restated as two, three, four, five, six or more immunogens)). The recombinant expression vector is constructed to include all appropriate regulatory sequences in frame with the respective nucleotide sequences encoding each immunogen such that each immunogen is expressed in the cell into which the recombinant expression vector is introduced. In certain specific embodiments, one or more of each additional immunogen (e.g., the second, third, fourth, fifth, sixth immunogen, etc.) may induce an immune response to the same designated antigen as the first immunogen. In other specific embodiments, each of the additional immunogens (e.g., the second, third, fourth, fifth, sixth immunogen, etc.) may induce an immune response specific for a different designated antigen (e.g., the second, third, fourth, fifth, sixth designated antigen etc.), respectively.

In other particular embodiments, methods for inducing an immune response are provided wherein the first immunogenic composition comprises at least one isolated/recombinant immunogen (for convenience, called a first immunogen) and may further comprise at least one additional isolated/recombinant immunogen. In other embodiments, the methods comprise administering a second immunogenic composition comprising a recombinant expression vector that encodes the first immunogen and encodes at least one additional immunogen. In a specific embodiment, the first immunogenic composition comprises at least two isolated/recombinant immunogens and the second immunogenic composition comprises a recombinant expression vector that contains a nucleotide sequence that encodes the at least two immunogens.

When two or more immunogens are included in the immunogenic composition comprising isolated/recombinant immunogens and/or are encoded by a polynucleotide sequence present in the recombinant expression vector, each immunogen may comprise amino acid sequences that include two different immunogenic regions or epitopes of a designated antigen of interest. At least one immunogen may comprise at least one B cell epitope or may comprise a T cell epitope or may comprise amino acid sequences that include both a B cell epitope and a T cell epitope. A second, different immunogen may comprise amino acid sequences that correspond to different B cell and/or T cell epitopes. When two or more immunogens are included in an immunogenic composition (or encoded by a recombinant expression vector), at least one immunogen comprises at least one T cell epitopic region. In more specific embodiments, at least one T cell epitopic region is capable of inducing a CD8 T cell specific immune response to the immunogen and the respective designated antigen.

In certain embodiments, when induction of an immune response specific for two or more immunogens is desired, at least one immunogen is capable of inducing an immune response that comprises at least a specific humoral and/or CD4 T cell response and at least one additional immunogen is capable of inducing an immune response that comprises at least a specific CD8 T cell immune response. Accordingly, provided herein in one embodiment is a method comprising administering to a subject in need thereof (a) an immunogenic composition (which may be called a first immunogenic composition) that comprises a first isolated/recombinant immunogen (which composition may further comprise an adjuvant) and (b) a second immunogenic composition that comprises a recombinant expression vector that encodes and directs expression of the first immunogen and a second immunogen, wherein at least the second immunogen is capable of inducing a specific CD8 T cell response. In certain embodiments, each of the at least two immunogens has the capability to induce an immune response to the same designated antigen. Alternatively, each of the at least two immunogens has the capability to induce an immune response specific for a different designated antigen (for convenience, also called the first and second designated antigens, etc. respectively).

In specific embodiments of the methods described herein, the two different immunogenic compositions are sequentially administered to the subject in need thereof. In one specific embodiment, the method comprises administering the immunogenic composition comprising the at least one isolated/recombinant immunogen (which may further comprise an adjuvant) prior to administration of the immunogenic composition that comprises the recombinant expression vector. Stated in another way, in certain embodiments, the method comprises administering an immunogenic composition comprising the recombinant expression vector subsequent to (i.e., after) administering the immunogenic composition comprising the isolated/recombinant immunogen (which composition may further comprise an adjuvant).

In other embodiments, the immunogenic composition comprising the recombinant expression vector is administered prior to the immunogenic composition comprising the isolated/recombinant immunogen (which composition may further comprise an adjuvant). Stated in another way, in certain embodiments, the method comprises administering the immunogenic composition comprising the at least one immunogen (which may further comprise an adjuvant) subsequent to (i.e., after) administering the immunogenic composition comprising the recombinant expression vector.

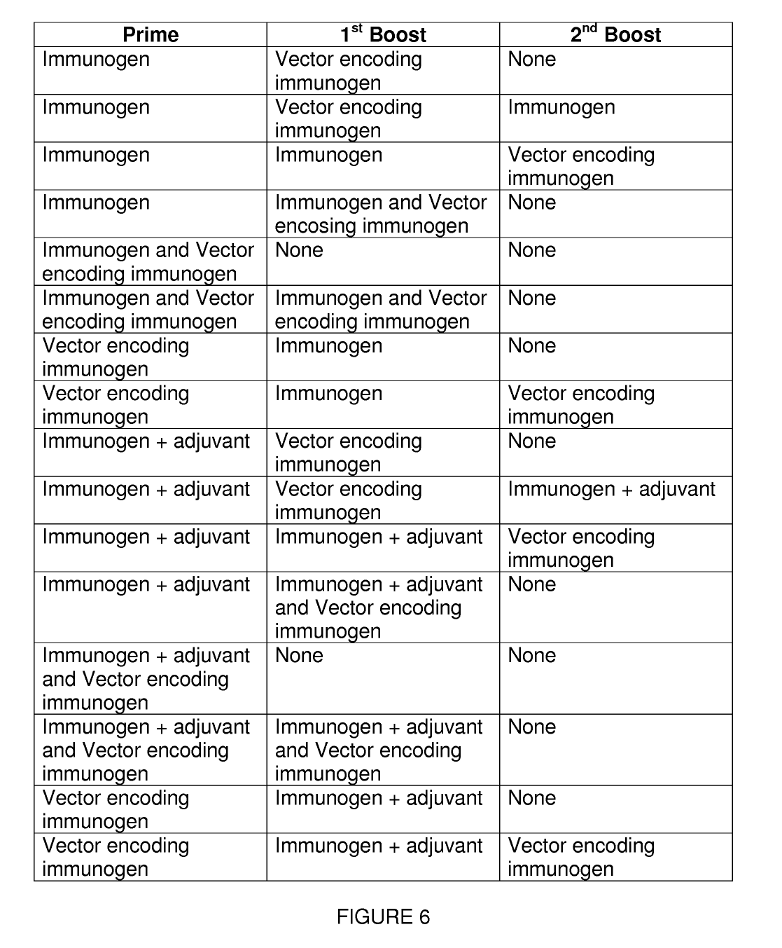

Inducing an immune response using the dual immunization methods and immunogenic compositions described herein may be accomplished by employing a variety of different immunization regimens. An exemplary, nonexhaustive list of immunization regimens is presented in FIG. 6 and FIG. 5. In particular, described herein is a planned clinical regimen for CMB305 in which ID-LV305 (a lentiviral vector as described herein, pseudotyped with an alphavirus modified to target dendritic cells, which lentiviral vector expresses NY-ESO-1) and IDC-G305 (recombinant NY-ESO-1 protein in combination with GLA-SE (a synthetic lipid A analog TLR4 agonist)) used in the CMB305 combination therapy will be administered sequentially at different times. In particular, LV305 is administered at day 0 and at day 21 followed by administration of G305 at day 35. Another dose of lentiviral vector is given at day 49 followed by another administration of G305 at day 63. A fourth dose of LV305 is given at day 77 followed by a third dose of G305 at day 91.

These and additional embodiments of the methods for inducing an adaptive, antigen-specific immune response are described in greater detail below and herein.

In specific embodiments, methods comprise administering the immunogenic composition comprising the isolated/recombinant immunogen (for ease of reference called a first immunogenic composition) and/or the immunogenic composition comprising the recombinant expression vector (for ease of reference called a second immunogenic composition) more than once to the subject. In particular embodiments, the immunogenic composition comprising the immunogen (which may further comprise an adjuvant) is administered at least two, at least three, at least four, at least five, or more times (e.g., twice (two times), three times, four times, five times, or more) to the subject. Stated another way, multiple doses (i.e., 2, 3, 4, 5, 6, or more doses) of the first immunogenic composition are administered to the subject. When the first immunogenic composition is administered multiple times (i.e., twice (two times), three times, four times, five times, or more), each administration of the first immunogenic composition may be sequential and each and all administrations of the first composition are prior to administration of the second composition. In other particular embodiments, the second composition is administered after one dose of the first composition and prior to a subsequent dose of the first composition. By way of example, when the first composition is administered two times to the subject, the second composition may be administered subsequent to the first administration (i.e., first dose) of the first immunogenic composition and prior to administration of the second administration (i.e., second dose) of the immunogenic composition. In another specific embodiment, such as when the first immunogenic composition is administered three times (i.e., three doses are administered), the second composition may be administered after the first dose and prior to the second dose; after the second dose and prior to the third dose; or after all three doses of the first immunogenic composition. In yet another specific embodiment, such as when the first immunogenic composition is administered four times (i.e., four doses are administered), the second composition may be administered after the first dose and prior to the second dose; after the second dose and prior to the third dose; after the third dose and prior to the fourth dose; or after all four doses of the first immunogenic composition. A person skilled in the art can readily appreciate that when five or more doses of the first immunogenic composition are administered, the second composition may be administered subsequent to any one of the multiple doses of the first immunogenic composition or subsequent to administration of all doses of the first immunogenic composition. In alternative embodiments, the second immunogenic composition is administered once and is administered prior to all administrations of the first immunogenic composition.

In still another embodiment, when the first immunogenic composition is administered multiple times (i.e., two or more times), one dose of the first immunogenic composition may be administered concurrently with administration of the second immunogenic composition. By way of example, when the dosing regimen comprises administering two doses of the first immunogenic composition, a first dose may be administered prior to concurrent administration of the second immunogenic composition and the second dose of the first immunogenic composition. By way of additional example, when the dosing regimen comprises administering three or more doses of the first immunogenic composition, at least one of the three doses is administered concurrently with administration of the second immunogenic composition and the additional doses of the first immunogenic composition may be administered prior to concurrent administration of both compositions, subsequent to concurrent administration of both compositions, or one or more doses may be administered prior to concurrent administration of both compositions and the remaining doses of the first immunogenic composition may be administered subsequent to concurrent administration of both compositions depending on the total number of doses of the first immunogenic composition intended to be administered according to the particular dosing regimen.

In certain particular embodiments, administration of the first immunogenic composition is administered two times and the second immunogenic composition is administered (a) subsequent to the first administration of the first immunogenic composition and prior to the second administration of the first immunogenic composition; (b) subsequent to the second administration of the first immunogenic composition; (c) prior to the first administration of the first immunogenic composition; or (d) concurrently with the first or the second administration of the first immunogenic composition. In another particular embodiment, the first immunogenic composition is administered three times and the second immunogenic composition is administered (a) subsequent to the first administration of the first immunogenic composition and prior to the second administration of the first immunogenic composition; (b) subsequent to the second administration of the first immunogenic composition and prior to the third administration of the first composition; (c) subsequent to the third administration of the first immunogenic composition; (d) prior to the first administration of the first immunogenic composition; or (e) concurrently with the first, the second, or the third administration of the first immunogenic composition. In still another particular embodiment, the first immunogenic composition is administered four times and the second immunogenic composition is administered (a) subsequent to the first administration of the first immunogenic composition and prior to the second administration of the first immunogenic composition; (b) subsequent to the second administration of the first immunogenic composition and prior to the third administration of the first composition; (c) subsequent to the third administration of the first immunogenic composition and prior to the fourth administration of the first composition; (d) subsequent to the fourth administration of the first immunogenic composition; (e) prior to the first administration of the first immunogenic composition; or (f) concurrently with the first, the second, the third, or the fourth administration of the first immunogenic composition.

In still other specific embodiments, methods are provided wherein the second composition (i.e., the immunogenic composition comprising the recombinant expression vector encoding at least one immunogen) is administered two times and the first immunogenic composition (i.e., the immunogenic composition comprising the at least one isolated/recombinant immunogen and which may further comprise an adjuvant) is administered once, two times, three times, four times, five times, or more. Each of the two administrations (i.e., two doses) of the second composition and each administration of the first immunogenic composition (i.e., the first, second, third, fourth, or fifth dosing) may be administered sequentially in any order. In other particular embodiments, at least one of the doses of the second immunogenic composition is administered concurrently with a dose of the first immunogenic composition.

In another particular embodiment, methods are provided wherein the second composition (i.e., the immunogenic composition comprising the recombinant expression vector, such as a lentiviral vector, encoding at least one immunogen) is administered two times and the first immunogenic composition (i.e., the immunogenic composition comprising the at least one isolated/recombinant immunogen and which may further comprise an adjuvant) is subsequently administered one time, followed by an additional administration of the second immunogenic composition, subsequently followed again by administration of the first immunogenic composition, followed again by another administration of the second immunogenic composition and an additional administration of the first immunogenic composition. In certain embodiments, this regimen may be followed by additional administrations of the first immunogenic composition as boosts.

As described herein, in other embodiments, the immunogenic composition comprising the isolated/recombinant immunogen (which composition may further comprise an adjuvant) and the immunogenic composition comprising a recombinant expression vector that comprises a nucleotide sequence encoding the immunogen may be administered concurrently at least once. In one such embodiment, methods are provided herein that comprise administering (1) an immunogenic composition comprising the immunogen (which composition may further comprise an adjuvant) and sequentially administering, in either order, (2) a second dose of the immunogenic composition comprising the immunogen concurrently with an immunogenic composition comprising a recombinant expression vector encoding the immunogen. In one particular embodiment, the immunogenic composition comprising the immunogen is administered prior to administering concurrently the immunogenic composition comprising the recombinant expression vector and the immunogenic composition comprising the immunogen (i.e., a second dose of the immunogenic composition comprising the immunogen). In another specific embodiment, the immunogenic composition comprising the immunogen is administered subsequent to concurrent administration of the immunogenic composition comprising a recombinant expression vector with the immunogenic composition comprising the immunogen. In still more particular embodiments, each dose of the immunogenic composition comprising a recombinant/isolated immunogen (i.e., first immunogenic composition) is administered concurrently with a dose of the immunogenic composition comprising the recombinant expression vector encoding the immunogen (i.e., second immunogenic composition). More specifically, methods are provided herein wherein a first dose of the first immunogenic composition is administered concurrently with a first dose of the second immunogenic composition (also called the priming immunization), followed by concurrent administration of a second dose of the first immunogen and a second dose of the second immunogenic composition (also called the boosting immunization). In certain embodiments, the subject may be immunized a third time by concurrent administration of the first and second immunogenic compositions. The time interval between the priming immunization and the boosting immunization(s) is discussed in greater detail herein and is selected on the basis of results from pre-clinical and/or clinical studies.

With respect to the methods described herein that include sequential administration of the immunogenic compositions, the time interval between doses can be readily determined by a person skilled in the art practicing clinical trials. The dosing regimen for human subjects may also be informed by results from pre-clinical studies and knowledge in the art. In certain embodiments, time interval between administration of doses of the immunogenic compositions may be at least one, two, three, four, five, six, or seven days or one, two, three, four, five, six, seven, or eight weeks, or may be at least one, two, three, four, five, six, seven, eight, nine, ten, or eleven months, or at least one, two, three, or four years. By way of illustration, when the immunogenic composition comprising a recombinant expression vector (for ease of discussion, called the second immunogenic composition) is administered subsequent to at least one dose of the immunogenic composition comprising the immunogen (for ease of discussion, called the first immunogenic composition), the second immunogenic composition is administered subsequent to administration of the at least one dose of the first immunogenic composition at any one of the time intervals described herein or that may be determined by appropriate preclinical and clinical studies.

Similarly, when the immunogenic composition comprising a recombinant expression vector (for ease of discussion, called the second immunogenic composition) is administered prior to at least one dose of the immunogenic composition comprising the immunogen (for ease of discussion, called the first immunogenic composition), the first immunogenic composition is administered subsequent to administration of the at least one dose of the second immunogenic composition at any one of the time intervals described herein or that may be determined by appropriate preclinical and clinical studies. By way of illustration, in one particular embodiment the second immunogenic composition may be administered at day zero, on or about day 21 (e.g., from about day 19 to about 23), on or about day 49 (e.g., from about day 47 to about day 51) and on or about day 77 (e.g., from about day 75 to about 79), while the first immunogenic composition may be administered on or about day 35 (e.g., from about day 33 to about day 37), on or about day 63 (e.g., from about day 61 to about 65) and again on or about day 91 (e.g., from about day 89 to about day 93).

In certain embodiments, the subject may be immunized a third, fourth, or fifth time with one or more of the immunogenic compositions. The time interval between a third immunization and the second immunization may be the same or different than the time interval between administrations of the first immunogenic composition and the second immunogenic composition or the time interval may be different. The time intervals as described herein between administrations of the same or different immunogenic compositions pertain to any of the administration regimens described herein (including, for example, the regimens illustrated in FIG. 6).

The immune response induced by administering the immunogenic compositions described herein according to the methods described above comprises an adaptive immune response that includes a humoral response and a cellular response (which comprises a CD4 immune response and a CD8 immune response) specific for each immunogen present in each immunogenic composition and thereby specific for the designated antigen respective to each immunogen. When the immunogenic composition comprising the isolated/recombinant immunogen (which composition may further comprise an adjuvant) and the immunogenic composition comprising a recombinant expression vector that comprises a nucleotide sequence encoding the immunogen are administered sequentially, at least the composition or compositions administered first (which may also be called the priming composition) are capable of inducing an immune response that comprises a CD4 T cell response specific for the immunogen and the respective designated antigen. The immune response induced by the priming composition may also comprise an antibody response specific for the immunogen and the respective designated antigen. The immunogenic composition(s) administered second (which may also be called a boosting composition) induces an immune response that comprises a CD8 T cell response specific for the immunogen and the designated antigen. Administration of the boosting composition may also induce or boost the antigen-specific antibody response and/or CD4 T cell specific immune response. In certain specific embodiments and as described herein, administration of an immunogenic composition comprising a recombinant expression vector that comprises a nucleotide sequence encoding an immunogen is capable of inducing an immune response that at least comprises inducing a CD8 T cell immune response specific for the immunogen and the respective designated antigen.

The immune response induced by the first administration (i.e., the first dosing) of an immunogenic composition described herein may comprise a humoral immune response and a CD4 T cell immune response, each specific for the immunogen included in the immunogenic composition. The first dosing may comprise administering an immunogenic composition comprising the isolated/recombinant immunogen (which may further comprise an adjuvant) or an immunogenic composition comprising the recombinant expression vector that encodes and directs expression of the immunogen, or the first dosing may comprise concurrent administration of each of the aforementioned immunogenic compositions. A second immunization (i.e., boosting immunization) includes administration of one or more of these immunogenic compositions and is capable of inducing an immune response that comprises a specific CD8 T cell immune response.

In particular embodiments, the immunogenic composition (also called a first immunogenic composition) that comprises the at least one isolated/recombinant immunogen (and which may further comprise an adjuvant) is capable of inducing an immune response that comprises a CD4 T cell response specific for the immunogen and thereby specific for the respective designated antigen, and which immune response may also comprise inducing a humoral response (i.e., specific antibody response or antigen-specific antibody response) to the immunogen. The other immunogenic composition (or second immunogenic composition) comprising the recombinant expression vector that comprises a nucleotide sequence encoding the immunogen is capable of at least inducing a CD8 T cell response specific for the immunogen and thus capable of inducing a CD8 T cell response specific for the designated antigen.

Accordingly, methods are provided for inducing a cytotoxic T cell response (CTL) comprising administering to the subject in need thereof, an immunogenic composition comprising at least one isolated/recombinant immunogen (which composition may further comprise an adjuvant) and sequentially and/or concurrently administering an immunogenic composition comprising a recombinant expression vector that comprises a nucleotide sequence encoding the immunogen. These methods may be performed according to any of the herein described steps of administration of the two immunogenic compositions, including the multiple dosing regimens. The CTL response is specific for a cell or particle that bears or presents the immunogen and/or respective designated antigen. In certain particular embodiments, and by way of example, when the immunogen is a tumor-associated antigen, the CTL response is specific for a tumor cell that expresses the immunogen and/or designated antigen. The immunogen and/or designated antigen may be present on the tumor cell surface and therefore accessible to cytotoxic T cells. The methods and compositions described in detail herein are therefore useful for reducing the likelihood of occurrence or recurrence of a tumor comprising a plurality of tumor cells that bear or express the tumor-associated antigen.

In other particular embodiments, the immunogen and designated antigen may be from an infectious disease microorganism, such as a virus, bacterium, parasite, or fungus, and the CTL immune response is specific for the virus, bacterium, parasite, or fungus, respectively, that expresses or bears the immunogen and/or designated antigen. The methods described herein are therefore useful for preventing or treating an infection caused by the respective infectious disease organism.

Also as described herein, in certain embodiments, these methods for inducing a CTL response may comprise administering a recombinant expression vector that is multicistronic and comprises a nucleotide sequence that encodes at least one additional immunogen (i.e., at least two, at least three, at least four, at least five, at least six, or more immunogens which may be restated as two, three, four, five, six or more immunogens)). In certain specific embodiments, upon expression of each of the additional immunogens (e.g., the second, third, fourth, fifth, sixth immunogen, etc.), each may induce an immune response to the same designated antigen as the first immunogen. In other specific embodiments, each of the additional immunogens (e.g., the second, third, fourth, fifth, sixth immunogen, etc.) may induce an immune response specific for a different designated antigen (e.g., the second, third, fourth, fifth, sixth etc.), respectively. In other certain embodiments, the immunogenic composition that comprises the at least one isolated/recombinant immunogen may comprise at least two isolated/recombinant immunogens (e.g., two, three, four, five, six or more immunogens) to form a multivalent immunogenic composition. In instances when the two or more immunogens are combined with an adjuvant, the immunogenic composition may comprise each immunogen formulated separately with an adjuvant and then the adjuvanated immunogens are combined to form the immunogenic composition that is administered to the subject. Alternatively, the two or more immunogens may be combined with an adjuvant and formulated together to form the immunogenic composition. In certain specific embodiments, each additional immunogen (e.g., the second, third, fourth, fifth, sixth immunogen, etc.) may induce an immune response to the same designated antigen as the first immunogen. In other specific embodiments, each additional immunogen (e.g., second, third, fourth, fifth immunogen, etc.) may induce an immune response specific for a different designated antigen (e.g., a second, third, fourth, fifth, sixth etc.), respectively.

In more specific embodiments for practicing the methods and uses described herein, an adjuvant, for example, a non-toxic lipid A-related adjuvant, may be formulated with the immunogen. In other specific embodiments, an adjuvant, such as a non-toxic lipid A-related adjuvant, may be administered in combination with an immunogenic composition comprising a recombinant expression vector that comprises a nucleotide sequence encoding the immunogen. In even more specific embodiments, the non-toxic lipid A-related adjuvant is GLA. In still more specific embodiments, GLA is formulated with SE to form a stable oil-in water emulsion (GLA/SE) for use in the methods and compositions described herein.

When an adjuvant is included in an immunogenic composition comprising the at least one isolated/recombinant immunogen, the adjuvant and immunogen are typically combined (i.e., formulated together, mixed) prior to administration to the subject. In alternative embodiments, the immunogenic composition comprising the at least one immunogen and the adjuvant may be administered separately but concurrently to the subject. When the immunogenic composition comprising the immunogen and the adjuvant are administered separately and concurrently, each of the immunogenic composition and the adjuvant may be administered at the same site via the same route or may be administered at the same site via different routes, or may be administered at different sites on the subject by the same or different administration routes. In certain embodiments, the adjuvant is a non-toxic lipid A-related adjuvant, such as GLA/SE.

When an adjuvant is included in an immunogenic composition comprising the recombinant expression vector, the adjuvant may be combined with (i.e., formulated together, mixed with) the recombinant expression vector (or vector particle comprising the recombinant expression vector) to form the immunogenic composition. In other embodiments, the immunogenic composition comprising the recombinant expression vector (or vector particle comprising the recombinant expression vector) are separate compositions, which may be administered at the same site via the same route or may be administered at the same site via different routes, or may be administered at different sites on the subject by the same or different administration routes. In certain embodiments, the adjuvant is a non-toxic lipid A-related adjuvant, such as GLA/SE.

In another specific embodiment, the immunization methods described herein for inducing a specific immune response comprise administering to the subject in need thereof an immunogenic composition comprising the adjuvant GLA/SE and an immunogen capable of inducing an immune response specific for a designated antigen. As described herein, GLA targets TLR4. TLR4 is unique among the TLR family in that downstream signaling occurs via both the MyD88- and TRIF-dependent pathways. Collectively, these pathways stimulate DC maturation, antigen processing/presentation, T cell priming, and the production of cytokines (e.g., IL-12, IFNα/β, and TNFα) (see, e.g., Iwasaki et al., Nat. Immunol. 5:987 (2004)).

In certain embodiments as described herein, the recombinant expression vector is incorporated into a vector particle, and methods described herein comprise administering an immunogenic composition that comprises the vector particle comprising a recombinant expression vector that encodes and directs expression of the immunogen. In more specific embodiments, the vector particle is a virus vector particle, such as a lentiviral vector particle. As described herein, the lentiviral vector particle may be DC-NILV, a self-inactivating, non-integrating lentivector that uses a modified Sindbis virus envelope glycoprotein to selectively enter dendritic cells (DCs). Upon vector entry into the DC, antigenic peptides generated via the active transcription and translation of the immunogen encoded by the vector are introduced into the MHC class I presentation pathway. Without wishing to be bound by any particular theory, use of DC-NILV generates robust CD8 T cell responses.

In one embodiment, the immunogenic composition comprising the recombinant expression vector or a vector particle comprising the recombinant expression vector is administered directly to the subject. In other specific embodiments, the target cell(s) may be isolated from a subject to whom the immunogenic composition will be administered, and the vector particle introduced into the target cells ex vivo. Then the targeted cells comprising the vector particle are introduced into the subject.

In even more specific embodiments, dual immunization methods comprise administering an immunogenic composition that comprises a recombinant/isolated immunogen(s) of interest is combined with the adjuvant GLA/SE. The methods further comprise administering a second immunogenic composition that comprises DC-NILV that encodes and expresses the immunogen(s). Exemplary but nonexhaustive immunization regimens for using these immunogenic compositions are shown in Table 1 below.

TABLE 1
Immunization Regimens

| Prime | 1^{st} Boost | 2^{nd} Boost |
|---|---|---|
| Immunogen(s) + | DC-NILV* | None |
| GLA/SE | | |
| immunogen(s) + | DC-NILV | immunogen(s) + |
| GLA/SE | | GLA/SE |
| immunogen(s) + | immunogen(s) + GLA/SE | DC-NILV |
| GLA/SE | | |
| immunogen(s) + | immunogen(s) + GLA/SE | None |
| GLA/SE | and DC-NILV | |
| immunogen(s) + | None | None |
| GLA/SE and | | |
| DC-NILV | | |
| immunogen(s) + | immunogen(s) + GLA/SE | None |
| GLA/SE and | and DC-NILV | |
| DC-NILV | | |
| DC-NILV | immunogen(s) + GLA/SE | None |
| DC-NILV | immunogen(s) + GLA/SE | DC-NILV |

*DC-NILV comprises a polynucleotide that encodes the immunogen(s).

Additional regimens are shown in FIG. 5 and FIG. 6.

The methods described herein are useful for inducing an immune response specific for any one of the immunogens and its respective designated antigen. As described in detail herein, a designated antigen of interest may be a tumor-associated antigen or an antigen from an infectious microorganism (e.g., a virus, bacterium, fungus, or a parasite). In certain particular embodiments, the methods described herein are useful for inducing an immune response specific for a tumor-associated antigen, including but not limited to a renal cell carcinoma antigen, a prostate cancer antigen, a mesothelioma antigen, a pancreatic cancer antigen, a melanoma antigen, a breast cancer antigen, a lung cancer antigen, and an ovarian cancer antigen. In more particular embodiments, the designated antigen of interest is a prostate cancer antigen, for example, prostatic acid phosphatase, prostate specific antigen, NKX3.1, or prostate specific membrane antigen. In another specific embodiment, compositions and methods are provided for immunizing a subject against a virus, such as HIV, CMV, a hepatitis virus, EBV, RSV, VSV, influenza, or HSV-2 or any other infectious virus described herein or in the art. Accordingly, the methods described herein may be used for inducing an immune response specific for a viral antigen. In more specific embodiments, the designated antigen of interest is an HSV-2 protein such as gD and UL19.

In certain embodiments, the dual immunization methods provided herein provide a method for priming and boosting a CD8 T cell immune response in a subject. The dual immunization methods provided herein may be used for inducing a cytotoxic T lymphocyte (CTL) response against a cell, a particle, or a microorganism bearing or expressing at least one designated antigen of interest. In particular embodiments, methods are provided herein for inducing a CTL response against a tumor cell that expresses at least one designated antigen of interest. In particular embodiments, the designated antigen (or a portion or portions thereof) is present on the outer cell surface of the tumor cell and exposed to the extracellular environment. The methods described herein are useful for reducing the likelihood of occurrence or recurrence (i.e., reducing the likelihood of occurrence or recurrence in a statistically, clinically, or biologically significant manner) of a tumor (which comprises a plurality of tumor cells) that bear, express, or secrete the tumor-associated antigen that is the designated antigen of interest.

In other particular embodiments, the methods described herein induce a CTL response against a microorganism, such as a virus, parasite, bacterium, or fungus cell. The designated antigen may be a microbial antigen that is typically secreted by the microorganism or may be a microbial antigen that is present on the cell surface of the microorganism and thereby has one or more immunogenic regions exposed and available for recognition by, and interaction with, molecules and cells of the subject's immune system. Accordingly, the methods described herein that comprise dual immunization of the subject are useful for treating and/or preventing (i.e., reducing the likelihood of occurrence of in a statistically, clinically, or biologically significant manner) a microbial infection, which would become exacerbated or would occur in the absence of administration of the immunogenic compositions described hereinto the subject.

As understood by a person skilled in the medical art, the terms, “treat” and “treatment,” refer to medical management of a disease, disorder, or condition of a subject (i.e., patient, host, who may be a human or non-human animal) (see, e.g., Stedman's Medical Dictionary). In general, an appropriate dose and treatment regimen provide the immunogen and optionally an adjuvant as detailed herein in an amount sufficient to provide therapeutic and/or prophylactic benefit. Therapeutic and/or prophylactic benefit resulting from therapeutic treatment and/or prophylactic or preventative methods include, for example an improved clinical outcome, wherein the object is to prevent or slow or retard (lessen) an undesired physiological change or disorder, or to prevent or slow or retard (lessen) the expansion or severity of such disease or disorder. Beneficial or desired clinical results from treating a subject include, but are not limited to, abatement, lessening, or alleviation of symptoms that result from or are associated the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can also mean prolonging survival when compared to expected survival if a subject were not receiving treatment. Subjects in need of the methods and compositions described herein include those who already have the disease or disorder as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder). The clinical benefit provided by the compositions (and preparations comprising the compositions) and methods described herein can be evaluated by design and execution of in vitro assays, preclinical studies, and clinical studies in subjects to whom administration of the compositions is intended to benefit. The design and execution of the appropriate preclinical studies and clinical studies can be readily performed by persons skilled in the relevant art(s).

The isolated/recombinant immunogens, recombinant expression vectors and/or vector particles may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human subject including a non-human mammalian subject.

With respect to administration of a recombinant expression vector, a therapeutically effective amount provides an amount of the polynucleotide which is capable of producing a medically desirable result (i.e., a sufficient amount of the immunogen is expressed to induce or enhance the immune response specific for the immunogen (humoral and/or cell-mediated response, including a cytotoxic T cell response) in a statistically, biologically, and/or significant manner) in a treated human or non-human animal. As is well known in the medical arts, the dosage for any one patient depends upon many factors, including the patient's size, body surface area, age, the particular compound to be administered, sex, time and route of administration, general health, and other drugs being administered concurrently. Doses will vary, but a preferred dose for administration of a vector particle comprising a recombinant expression vector is sufficient to provide approximately 10⁶ to 10¹² copies of the vector polynucleotide molecule (also referred to as vector genomes).

Pharmaceutical compositions, including the immunogenic and adjuvant compositions described herein, may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provides the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

In general, the amount of an immunogen, including fusion polypeptides as described herein, present in a dose, or produced in situ by an encoding polynucleotide present in a dose, ranges from about 0.01 μg to about 1000 μg per kg of host. The use of the minimum dosage that is sufficient to provide effective therapy is usually preferred. Patients may generally be monitored for therapeutic or prophylactic effectiveness using assays suitable for the condition being treated or prevented, which assays will be familiar to those having ordinary skill in the art and which are described herein. When administered in a liquid form, suitable dose sizes will vary with the size of the patient, but will typically range from about 1 ml to about 500 ml (comprising from about 0.01 μg to about 1000 μg per kg) for a 10-60 kg subject. Optimal doses may generally be determined using experimental models and/or clinical trials. The optimal dose may depend upon the body mass, body area, weight, or blood volume of the subject. As described herein, the appropriate dose may also depend upon the patient's (e.g., human) condition, that is, stage of the disease, general health status, as well as age, gender, and weight, and other factors familiar to a person skilled in the medical art.

Pharmaceutical compositions may be formulated for any appropriate manner of administration, including, for example, topical, oral, enteral, nasal (i.e., intranasal), inhalation, intrathecal, rectal, vaginal, intraocular, subconjunctival, sublingual, intradermal, intranodal, intratumoral, transdermal, or parenteral administration, including subcutaneous, percutaneous, intravenous, intramuscular, intrasternal, intracavernous, intrameatal or intraurethral injection or infusion. Methods of administration are described in greater detail herein.

For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

An immunogenic composition comprising a recombinant/isolated immunogen and an immunogenic composition comprising the recombinant vector construct or the vector particle may be formulated for delivery by any route that provides an effective dose of the immunogen. Such administrations methods include oral administration or delivery by injection and may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

For pharmaceutical compositions comprising a nucleic acid molecule such as the recombinant expression vectors described herein, the nucleic acid molecule may be present within any of a variety of delivery systems known to those of ordinary skill in the art, including nucleic acid, and bacterial, viral and mammalian expression systems such as, for example, vector particles and recombinant expression constructs as provided herein. Techniques for incorporating a polynucleotide (e.g., DNA) into such expression systems are well known to those of ordinary skill in the art. In other certain embodiments, the recombinant expression vector, which is typically DNA, may also be “naked,” as described, for example, in Ulmer et al., Science 259:1745-49 (1993) and reviewed by Cohen, Science 259:1691-92 (1993). The uptake of naked DNA may be increased by coating the DNA onto biodegradable beads, which are efficiently transported into the cells.

Nucleic acid molecules may be delivered into a cell according to any one of several methods described in the art (see, e.g., Akhtar et al., Trends Cell Bio. 2:139 (1992); Delivery Strategies for Antisense Oligonucleotide Therapeutics, ed. Akhtar, 1995, Maurer et al., Mol. Membr. Biol. 16:129-40 (1999); Hofland and Huang, Handb. Exp. Pharmacol. 137:165-92 (1999); Lee et al., ACS Symp. Ser. 752:184-92 (2000); U.S. Pat. No. 6,395,713; International Patent Application Publication No. WO 94/02595); Selbo et al., Int. J. Cancer 87:853-59 (2000); Selbo et al., Tumour Biol. 23:103-12 (2002); U.S. Patent Application Publication Nos. 2001/0007666, and 2003/077829). Such delivery methods known to persons having skill in the art, include, but are not restricted to, encapsulation in liposomes, by iontophoresis, or by incorporation into other vehicles, such as biodegradable polymers; hydrogels; cyclodextrins (see, e.g., Gonzalez et al., Bioconjug. Chem. 10:1068-74 (1999); Wang et al., International Application Publication Nos. WO 03/47518 and WO 03/46185); poly(lactic-co-glycolic)acid (PLGA) and PLCA microspheres (also useful for delivery of peptides and polypeptides and other substances) (see, e.g., U.S. Pat. No. 6,447,796; U.S. Patent Application Publication No. 2002/130430); biodegradable nanocapsules; and bioadhesive microspheres, or by proteinaceous vectors (International Application Publication No. WO 00/53722). In another embodiment, the nucleic acid molecules can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives (see also, e.g., U.S. Patent Application Publication No. 2003/0077829).

In particular embodiments of the methods described herein, the subject is a human or non-human animal. A subject in need of the treatments described herein may exhibit symptoms or sequelae of a disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The compositions provided herein can be in various forms, e.g., in solid, liquid, powder, aqueous, or lyophilized form. Examples of suitable pharmaceutical excipients and carriers for administering a vector particle, including a viral vector particle and a bacterial vector particle, immunogenic compositions, and recombinant expression vectors are known in the art. Such excipients, carriers, and/or additives can be formulated by conventional methods and can be administered to the subject at a suitable dose. Stabilizing agents such as lipids, nuclease inhibitors, polymers, and chelating agents that may be included in the compositions described herein can assist preservation of the compositions and components of the compositions from degradation within the body.

The vector particles, including a viral vector particle and a bacterial vector particle, immunogenic compositions, adjuvant compositions, and recombinant expression vectors provided herein can be packaged as kits. Kits can optionally include one or more components such as instructions for use, devices, and additional reagents, and components, such as tubes, containers and syringes for practice of the methods. Exemplary kits can optionally include instructions for use, a device or reagents for detecting a vector particle, the recombination expression vector, or the immunogen in a subject, and a device for administering the composition or compositions to a subject.

Kits comprising polynucleotides encoding an immunogen are also contemplated herein. Such a kit may also include at least one plasmid that encodes virus packaging components and a vector encoding Sindbis virus E2 glycoprotein variant. Some kits will contain at least one plasmid encoding virus packaging components, a vector encoding Sindbis virus E2 glycoprotein variant, and a vector encoding at least one DC maturation factor.

Kits comprising a viral vector encoding a sequence of interest (typically encoding an antigen or immunogen) and optionally, a polynucleotide sequence encoding a DC maturation factor are also contemplated herein. In some kits, the kit includes at least one plasmid encoding virus packaging components and a vector encoding Sindbis virus E2 glycoprotein variant.

A kit may also contain instructions. Instructions typically describe methods for administration, including methods for determining the proper state of the subject, the proper dosage amount, and the proper administration method, for administering the composition. Instructions can also include guidance for monitoring the subject over the duration of the treatment time.

Kits provided herein also can include devices for administration of each of the immunogenic compositions described herein and/or for administration of an adjuvant composition to a subject. Any of a variety of devices known in the art for administering medications, immunogenic compositions, and vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a composition is compatible with the active components of the kit. For example, a needle-less injection device, such as a high pressure injection device can be included in kits with vector particles, polynucleotides, and polypeptides not damaged by high pressure injection, but is typically not included in kits that include vector particles, polynucleotides, and polypeptides that may be damaged by high pressure injection.

Other embodiments and uses will be apparent to one skilled in the art in light of the present disclosures. The following examples are provided merely as illustrative of various embodiments and shall not be construed to limit the invention in any way.

### EXAMPLES

### Example 1

### Neutralizing Antibody Responses Against ID-VP02 can be Detected after Immunization

VP02 (also referred to elsewhere as DC-NILV) is a lentivector (LV)-based vaccine platform, which has been engineered to deliver tumor antigen-encoding genes to DCs in vivo. This vaccine platform has been shown to transduce DCs through pseudo-typing with engineered Sindbis virus (SINV) glycoproteins that bind the C-type lectin receptor DC-SIGN. As a result, the vector induces a high magnitude of functional CD8 T-cell immune responses after a single immunization in mice. The VP02 lentiviral vector platform is devoid of all HIV accessory proteins except for Rev (which facilitates the nuclear export of genomic transcripts during vector production), and the vector is encoded by a split genome with an extended deletion in the U3 region (ΔU3). The ΔU3 deletion is a self-inactivating mutation that: (1) prevents transcription of the full-length vector genome from reversed-transcribed dsDNA vectors in the infected target cell and (2) minimizes the risk of insertional activation that can occur when a 3′LTR can function as a promoter after integration. Specific mutations have been made in the vector and manufacturing process to: (1) enhance the ability of VP02 to transduce human DCs, and (2) provide redundant safety mechanisms to eliminate integrase enzyme-dependent integration events. In particular, the VP02 platform contains two specific modifications to enhance binding and entry into DCs. The first is the use of an alphavirus envelope glycoprotein with binding specificity to the DC-SIGN membrane protein. This envelope (SINVar1) has been genetically modified from the prototype to further increase its DC tropism and prevent binding to ubiquitous heparan sulfate receptors. In addition to these genetic modifications, ID-VP02 is produced in the presence of a small molecule inhibitor of mannosidase I, which results in the generation of ID-VP02 envelope glycoproteins with terminal high mannose residues (see e.g., WO2013/149167).

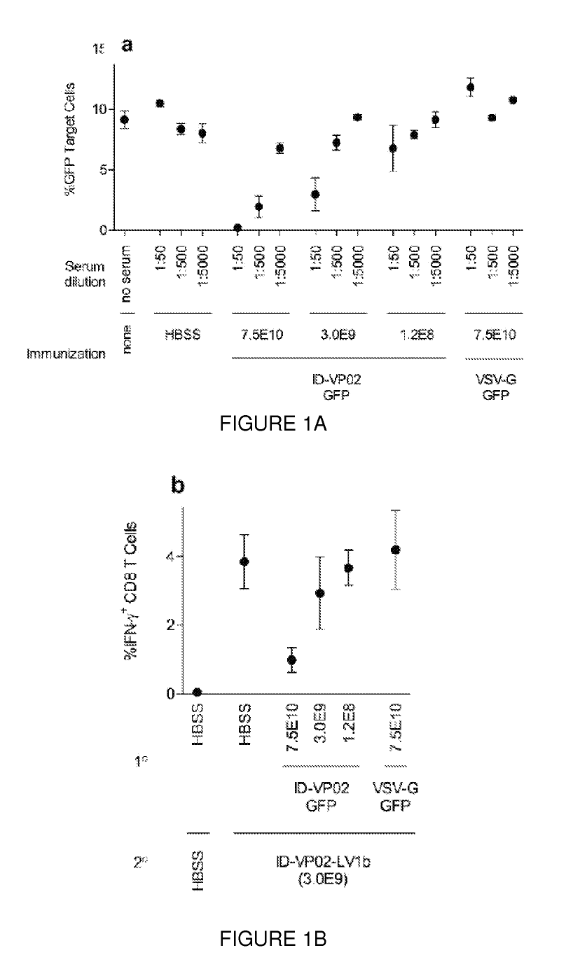

It is expected that a neutralizing antibody response would be generated against ID-VP02 after the first administration that could impact the potency of the vector for delivering antigen for presentation to CD8 T cells. To determine if neutralizing antibodies are generated against ID-VP02 after in vivo administration, serum was isolated from groups of three mice that had been immunized with either 7.5×10¹⁰, 3.0×10⁹, or 1.2×10⁸ vector genomes of ID-VP02 encoding GFP, 7.5×10¹⁰ vector genomes of VSV-G pseudotyped lentivector encoding GFP, or with HBSS. The serum was then evaluated for the presence of neutralizing antibodies against ID-VP02 by in vitro transduction assay. Serum from mice injected with either high- or mid-dose ID-VP02 was able to neutralize vector in this assay (FIG. 1A). The neutralizing antibody response was dose-dependent and specific to the Sindbis virus-derived envelope of ID-VP02, in that no serum neutralization was observed from mice immunized with the VSV-G control vector at the highest vector dose (7.5×10¹⁰ vector genomes).

Because anti-ID-VP02 antibodies were detected in this assay, we wanted to determine if the level of neutralizing antibodies present in ID-VP02 immunized mice would have an impact on the in vivo potency of the vector. As the immunological requirements to effectively prime naïve CD8 T-cells are much more stringent than those for boosting pre-existing memory cells, we sought to determine what level of previous exposure to ID-VP02 (i.e. dose) would be required to significantly impair the ability of a midrange-dose of ID-VP02 to prime naïve CD8 T-cells. Thus, the mice described above that had been immunized with high-, mid-, or low dose ID-VP02 encoding GFP, high-dose VSV-G pseudotyped lentivector encoding GFP, or HBSS were immunized with 3.0×10⁹ vector genomes of ID-VP02 encoding an alternative antigen cassette, termed LV1b, that encodes the minimal OVA₂₅₇ peptide sequence. The OVA₂₅₇-specific CD8 T-cell response in the spleen was measured by ICS on day 12 post-immunization. Relative to the CD8 T-cell responses observed in the control mice, there was a clear reduction in the ability of 3.0×10⁹ vector genomes of ID-VP02 to prime naïve CD8 T-cell responses within the context of previous exposure to a 25-fold higher dose (7.5×10¹⁰ vector genomes) of ID-VP02 encoding GFP (FIG. 1B). However, this reduction was not observed in animals that had been pre-exposed to equal (3.0×10⁹) or 25-fold lower (1.2×10⁸) doses of ID-VP02-GFP. As in the in vitro neutralization assay, the mechanism responsible for the reduction in vector potency appeared to be specific to the envelope of ID-VP02, in that no reduction in priming of naïve CD8 T cell was observed in mice previously immunized with 7.5×10¹⁰ vector genomes of VSV-G pseudotyped control vector.

These data, indicate that although vector-specific immunity can be generated against ID-VP02 at high-doses, the application of ID-VP02 is not limited to a single administration when equivalent mid-range doses are given for both the prime and boost immunizations.

### Example 2

### GLP Safety/Toxicity Study of CMB305 in Mice

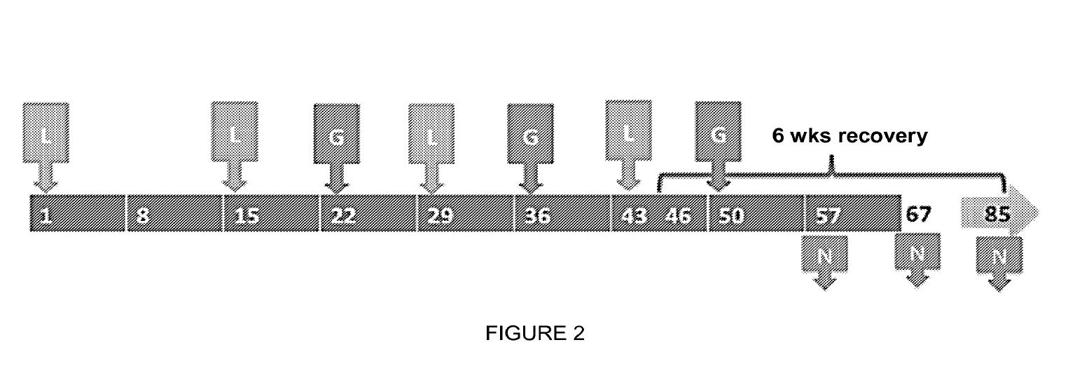

A formal GLP safety/toxicity study of the CMB305 investigational regimen is being conducted in mice. This study, entitled “A 13-week repeat dose toxicity study of CMB305 administered sequentially by intramuscular and subcutaneous injections for 8 weeks to BALB/c mice with a 17-day or 5 week recovery period” is currently in progress, and preliminary results are provided below. CMB305 is an investigational therapeutic vaccine for the treatment of cancer. This investigational treatment regimen is comprised of the two investigational products, ID-LV305 (a lentiviral vector based on the VP02 platform that expresses the NY-ESO-1 cancer-associated antigen) and IDC-G305 (recombinant NY-ESO-1 protein in combination with the synthetic TLR4 agonist GLA-SE), which are administered sequentially but via different routes of administration. In this study, 10 male and ten female BALB/c mice per necropsy time point were administered four doses of ID-LV305 s.c. (base of tail) and three doses of IDC-G305 i.m. (Table 2). A schematic of the study design is shown in FIG. 2. The dosage levels for ID-LV305 and IDC-G305 were 2.5×10⁸ vector genomes and 5 μg GLA-SE, respectively Animals in a control arm of the study received injection vehicles instead. The routes of the administrations were chosen because they are common routes for vaccine immunization and because these routes provide support for the intended administration to humans (i.d. and i.m.).

The objectives of this study are to evaluate the potential local and systemic toxicity of CMB305 when administered to male and female BALB/c mice by i.m. and s.c. injections for an 8-week dosing period, and to evaluate the reversibility, persistence, or delayed occurrence of any effects of CMB305 after a 17 day or 5-week recovery period.

TABLE 2
Pivotal GLP Safety/Toxicity Study Design

| | | | | Number of Animals/Necropsy | | | | | |
|---|---|---|---|---|---|---|---|---|---|
| | | Total | | | | Interim | | | |
| | | Injection | | Terminal | | Recovery | | Recovery | |
| | | Volume | | (Day 57) | | (Day 67) | | (Day 85) | |
| Group | Treatment | (μL) | Dose Level | M | F | M | F | M | F |
| 1 | Vehicle G^{a} | 50 | — | 10 | 10 | 10 | 10 | 10 | 10 |
| | Vehicle LV^{b} | 50 | — | | | | | | |
| 2 | IDC-G305^{a} | 50 | 5 μg NY-ESO-1 + | 10 | 10 | 10 | 10 | 10 | 10 |
| | | | 5 μg GLA-SE | | | | | | |
| | ID-LV305^{b} | 50 | 2.5 × 10⁸ genomes | | | | | | |

M = Male;
F = Female;
Vehicle G = IDC-G305 vehicle;
Vehicle LV = ID-LV305 vehicle
^{a}Dosed by IM injection in the quadriceps muscle of alternating hind limbs (beginning on the left).
^{b}Dosed by SC injection at the tail base

### Preliminary Results

The study is currently ongoing; the immunizations have been completed and the two first necropsies have been carried out.

### Clinical Pathology

Hematology:

There were no test article-related effects among hematology parameters in either sex. All mean and individual values were considered within an acceptable range for biological variation.

Clinical Chemistry:

There were no test article-related effects among clinical chemistry analytes in either sex. All mean and individual values were considered within an acceptable range for biological variation despite occasional mean values which were outside of the historical range at MPI Research. These were not considered meaningful based on their small magnitude, direction of change, and minimal animals affected including control vehicle.

### Mortality/Morbidity

Three animals were euthanized in extremis during the study.
Animals 2009 (Group 2 male) and 2013 (Group 2 male)—penile prolapses (dry and erythemic), body weight loss, mild-moderately dehydrated. Euthanized day 15 due to penile prolapse.
Animal 2020 (Group 2 male)—bodyweight loss, decreased activity, hunched posture, skin cold to the touch. Euthanized day 14.

An independent toxicology consultant concluded that animals were below the body weight range as specified in the protocol during the acclimatization period and one animal was found dead. However, three days prior to dosing, all animals were within the recommended body weight range even though in the low normal range and considered to be normal. The consultant contacted a qualified veterinarian (MSc, DVM, DACVP) to discuss this finding and he agreed that it was most probably due to the poor condition of the animals, even though a test article-related event can never be completely dismissed until the whole data set is made available at the completion of the study.

Upon review of the data available as of study day 15, the consultant made the following recommendations and comments:
1) Monitor animals more frequently by increasing the body weight and food consumption measurements from once weekly to twice weekly.
2) Provide water bottles to the animals in addition to the water system in place. It was noted that the contractor provided diet enrichment in the form of hydrogel to all animals and that the general condition of animals did improve with this addition. The food consumption data will be affected by this addition.
3) If body weights did continue to decrease post-second dose, the Sponsor should consider postponing treating mice with G305. The Sponsor was provided body weight data from Days 16 and 17. The male mice were gaining weight on both days post the 2^{nd} dose of LV-305, thus no postponement of dosing G305 would be necessary.
4) The consultant cautioned the Sponsor regarding interpretation of the results of the clinical pathology data reported on the two moribund mice, as these data can be highly variable in these animals. Historical control data was requested from the contractor and they mentioned that the clinical pathologist would review this data.
5) The finding of the penis prolapse was probably due to the poor condition of these animals and probably not test article-related.

Subsequent clinical findings and weight measurements have been acceptable in all mice in all groups.

### Immunogenicity Results

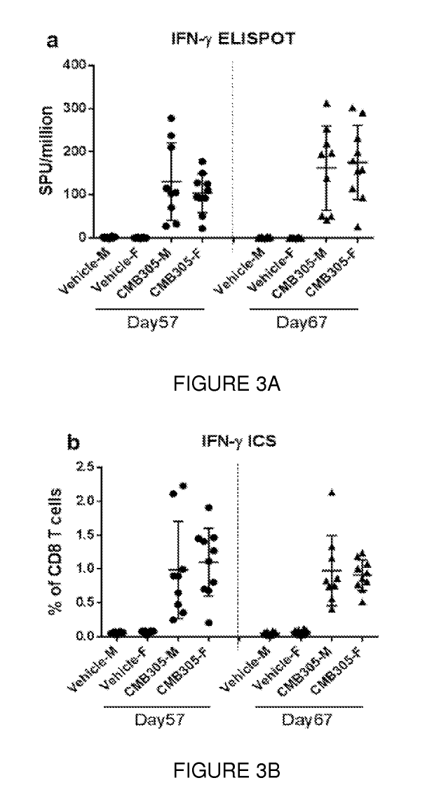

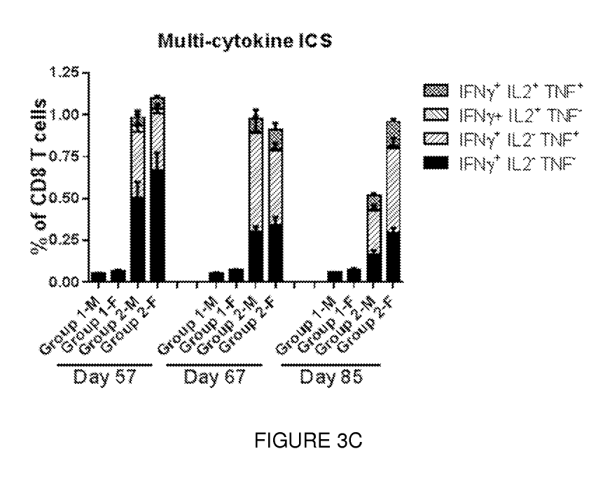

In order to include immune response levels as parameters in the pivotal GLP safety/toxicity study of CMB305 carried out in BALB/c mice see above and Table 2), splenocytes were harvested at the time of study necropsies, then shipped fresh to our facility for processing and assessment of NY-ESO-1 specific CD8 T-cell responses. As seen in FIG. 3, intracellular cytokine staining followed by flow cytometry demonstrate that significant immune responses were induced in mice that received the combined dosing regimen of CMB305, whereas mice immunized with formulation vehicles alone did not display such responses.

### Example 3

### Immunogenicity of ID-LV305 in B6D2F1 Mice Administered Intradermally at 1 or 4 Lymph Node Sizes and Various Dosage Levels

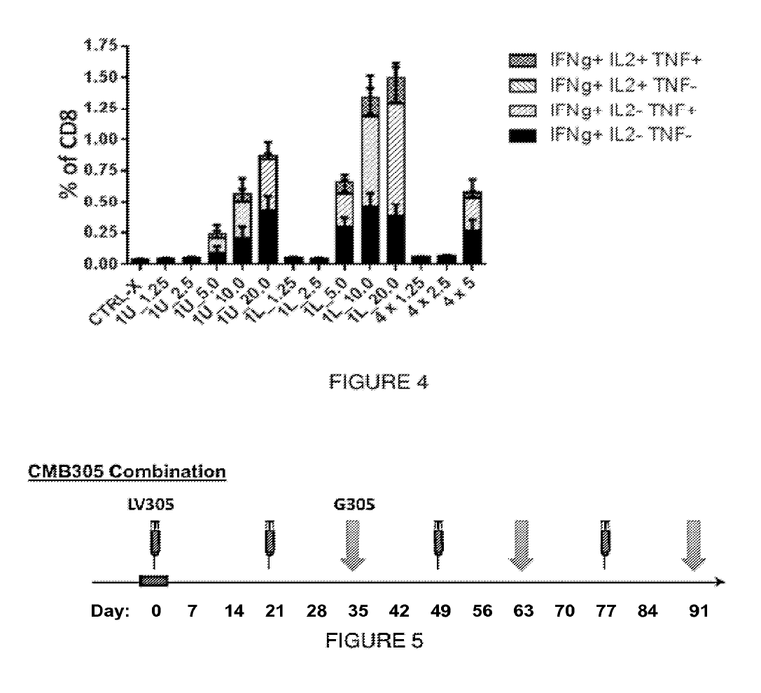

In order to determine the lowest immunogenic dosage level of ID-LV305 capable of inducing T-cell responses against NY-ESO-1 following administration at one versus four lymph node drainage sites intradermally, B6D2F1 mice were immunized with ID-LV305 at doses ranging from 1.25×10⁸ to 2.0×10⁹ vector genomes at 2-fold increments. The sites used were contralaterally laterally positioned at the left and right sides of the upper back and lower back, close to the tail base (FIG. 4) Animals were immunized once and NY-ESO-1-specific T-cell responses were assessed in splenocytes 14 days later. As shown in FIG. 4 (bottom panel), the lowest immunogenic dose when administered as a single bolus injection at either the upper or lower intradermal site was 5.0×10⁸ vector genomes. A trend towards increasing immune response levels was observed as the dosage levels were increased. However, following administration of ID-LV305 split into four separate injections, the lowest total immunogenic dose was 2.0×10⁹ vector genomes (5.0×10⁸ vector genomes administered to each of the four injection sites) Similar results, albeit with lower response levels, were seen for antigen-specific CD4 T-cell responses (not shown). These results were surprising because it was expected that the effect of multiple injections would be additive. However, these results indicate that there appear to be factors that reduce the expected additive effect from the multiple injections. The results of this experiment indicate that administration to multiple injection sites can be used to increase the total injection volume. These results indicate that a lower immunogenic dose can be used when administered as a single injection than when multiple administrations is used. A regimen involving a single injection could improve the convenience and lower the dose required for the best clinical effect. This results in lower cost per treatment and improved patient experience as fewer injections are required.

### Example 4

### Evaluating the Safety, Tolerability and Immunogenicity of Intradermally Administered VP02-NY-ESO-1 in Patients with Locally Advanced or Metastatic Cancer Expressing NY-ESO-1

The overall goal of the proposed first-in-human study is to evaluate the safety and immunogenicity of repeat dose VP02-NY-ESO-1 administration in patients with NY-ESO-1 expressing cancer. Additional objectives include evaluations of clinical tumor responses and tumor histologic response obtained at biopsy.

Patients will be enrolled only if they have locally advanced or metastatic cancer expressing NY-ESO-1 by RT-PCR and/or IHC and/or the presence of serum antibodies to NY-ESO-1 and have not responded favorably to one or more prior systemic therapies (at least two or more systemic therapies for NSCLC and breast cancer). Patients should not have bulky and rapidly progressive disease. Patients who have one of the following cancer histologies can be enrolled: breast cancer, melanoma, NSCLC, ovarian cancer, renal cell carcinoma or sarcoma. These tumor types were selected based on their known frequency of NY-ESO-1 expression (See e.g., CTDatabase website at cta.lncc.br/index.php) and includes tumors known to be responsive to immunotherapy. Although approved treatment options continue to increase for patients with many different types of malignancies, patients with advanced cancer continue to have a poor prognosis and current therapies are largely unsatisfactory. Patients will be fully informed regarding the potential risks and benefits of the proposed investigational therapy. Adequate hematologic and metabolic function will be required criteria for study entry.

As vaccine and vector dosing selection for clinical studies commonly does not adjust for allometric scaling across species, and because this is a first-in-human clinical trial, the proposed human starting dose level (5×10⁸ genomes) is about the same as the highest dose that was used in the BALB/c mouse GLP toxicology study. Adjusting for body weights, the starting human dose is approximately 2800-fold lower than the highest dose from the mouse GLP toxicology study. The proposed two-log dose escalation range reflects consideration of the safety margins from non-GLP animal toxicology studies and conventions from prior vector immunotherapy clinical trials.

VP02-NY-ESO-1 will be administered by i.d. injection in 8 sites once every 3 weeks for 3 doses. Non clinical studies suggest that injections every 2-4 weeks generate the most durable and highest magnitude immune responses.

### Procedure

VP02-NY-ESO-1 is administered at Day 0, 21, and 42 in a 3+3 sequential dose escalation design. All dose levels of VP02-NY-ESO-1 will be administered in 1 mL as 8×125 μL i.d. injections, two over each deltoid and two over each quadriceps at least 10 cm distal to inguinal lymph nodes. Intradermal injections should be located at least 3 cm apart at each site.

Three dose level cohorts will be treated contingent upon absence of DLT and acceptable safety data review for the preceding two cohorts.

TABLE 3
Protocol ID-VP02-NY-ESO-1-2013-001: Cohorts

| Cohort | Dose Level of ID-VP02-NY-ESO-1 |
|---|---|
| 1 | 5 × 10⁸ vector genomes |
| 2 | 5 × 10⁹ vector genomes |
| 3 | 5 × 10¹⁰ vector genomes |

### Example 5

### Summary of Initial Phase 1 Data of VP02-NY-ESO-1 Administration

Below is a high-level summary of the initial data from the Phase 1 clinical trial outlined in Example 4.

Favorable safety profile with only Grade 1 or 2 adverse events observed in the twelve patients treated in this dose-escalation trial evaluating three different dose levels of LV305. CD4 or CD8 specific T-cell responses were observed after therapy in eight of eleven (73%) evaluable patients. Four of the six patients treated with the mid or high dose levels of LV305 developed de novo CD8 T-cell responses against NY-ESO-1. As expected, the therapy had no effect on anti-NY-ESO-1 antibody levels. Of the twelve patients with various types of soft tissue sarcoma expressing NY-ESO-1 who were enrolled and treated: Four of six patients with evidence of tumor growth prior to LV305 treatment stabilized and stopped progression with the longest at 347+ days; tumor regression up to 13.8% was observed in one patient. Eight of twelve (67%) patients achieved a best response of stable disease (SD) with a median duration of 208 days (range: 139-347+) and the progression-free rate (PFR) of all twelve patients at six months was at least 42%¹. Although a small study and differences in the patient population exist, the observed PFR of at least 42% compares favorably to the historical PFR in the analysis of a large group of patients reported by Van Glabbeke, et al.², where active agents for first- and second-line treatment exhibited a PFR of >30-56% (histology dependent) and >14% at six months, respectively (importantly, the LV305 study consisted of patients who had all received at least one prior treatment). ¹Note that the total tumor burden in the LV305 study was restricted to <10 cm and ECOG status was <2;²Van Glabbeke, et al., European Journal of Cancer, “Progression-free rate as the principal end-point for phase II trials in soft-tissue sarcomas”, 2002.

Conclusions:

LV305 demonstrated acceptable safety at all doses up to 10₁₀ vg. At the lowest dose, LV305 generated strong T cell responses and preliminary evidence of anti-tumor effect. Data from the mid and high dose are pending. These encouraging results will be followed by studies of LV305 (10₁₀ vg) alone, in prime/boost with G305 (NY-ESO-1 protein-TLR4 agonist) and with anti-PD-1 therapy.

### Example 6

### CMB305 Combination Clinical Protocol

This clinical trial will be conducted following the dose-escalation phase (Part 1) and will enroll patients with melanoma, ovarian cancer (including fallopian tube carcinoma), sarcoma (specifically: synovial sarcoma or myxoid/round cell liposarcoma types), and NSCLC to receive either ID-LV305 alone or CMB305 (sequential administration/combination of ID-LV305 and IDC-G305). In the CMB305 part of the trial, patients will be assigned to receive CMB305 using the schedule outlined in FIG. 5. The dose of ID-LV305 will consist of the established safe dose determined during Part 1 Dose Escalation and the dose of IDC-G305 will consist of the established safe dose of GLA and 250 ug of NY-ESO-1 protein. In CMB305, 4 doses of ID-LV305 i.d. and 3 doses of IDC-G305 intramuscular are administered.

GLA is a fully synthetic TLR4 agonist, which has been examined in over 900 patients and healthy volunteers as an adjuvant with NY-ESO-1 protein and with infectious agent antigens such as in flu vaccines. GLA has an established safety profile and the 5 ug dose level has been examined in several hundred patients or healthy volunteers. In an ongoing separate Phase 1 trial of IDC-G305, the 5 ug dose of GLA plus 250 ug of NY-ESO-1 protein is the intermediate (mid) dose level and has already been determined to be safe. A higher Mug dose cohort is currently being examined in this Phase 1 trial.

For the CMB305 treatment arm, the dose of GLA will consist of 5 ug GLA and will be formulated in combination with 250 ug of NY-ESO-1 protein. The dose of GLA may be increased to Mug should that dose be determined to be safe in a separate Phase 1 IDC-G305 safety study. As shown in FIG. 5, the ID-LV305 and IDC-G305 used in the CMB305 combination therapy will be administered sequentially at different times. Each component was determined to be well tolerated in Phase 1 studies.

CMB305 will initially enroll 3 patients. To establish the safety of the sequential combination, the first 3 patients receiving CMB305 will be observed for treatment emergent DLTs arising during the first 49 days of therapy. Following the observation period, all SAE and DLT safety events deemed potentially related to the study agents will be reviewed by the Sponsor and the independent DMC.

Peripheral blood will be drawn for immunogenicity assays at baseline and then at several timepoints prior to treatment with ID-LV305 or IDC-G305, and then 3 weeks following completion of all planned treatments on Day 112 for CMB305. Leukapheresis will be performed at baseline and following the final CMB305 administration (within Days 98 to 112) to collect cells for exploratory biomarkers and immunologic assays.

Tumor biopsies will be performed in a subpopulation of consenting patients with accessible tumors about three weeks after the completion of all planned treatments. Tumor tissue from any pre-study tumor biopsy or tumor surgical excision specimen may be used as the baseline biopsy, including specimens evaluated for NY-ESO-1 expression as part of another study.

Tumor imaging will be performed during the screening visit (baseline). Patients will continue with imaging and clinical assessment about every 8 weeks until disease progression as defined by the irRC. Blood samples will be collected after initial injection for an assay to test for persistence of ID-LV305. During CMB305, bloods will be collected at baseline and then at visits on Day 168, Day 224, 12, and 24 months. Samples will be assayed for presence of the viral genome by PCR. Depending on the results through 12 months, annual assessments may continue until 2 consecutive samples show no evidence of ID-LV305 persistence.

### Example 7

### Anti-Tumor Response Following Prophylactic Vaccinations with VP02-NY-ESO-1 in the Mouse CT26-NY-ESO-1 Subcutaneous Tumor Model

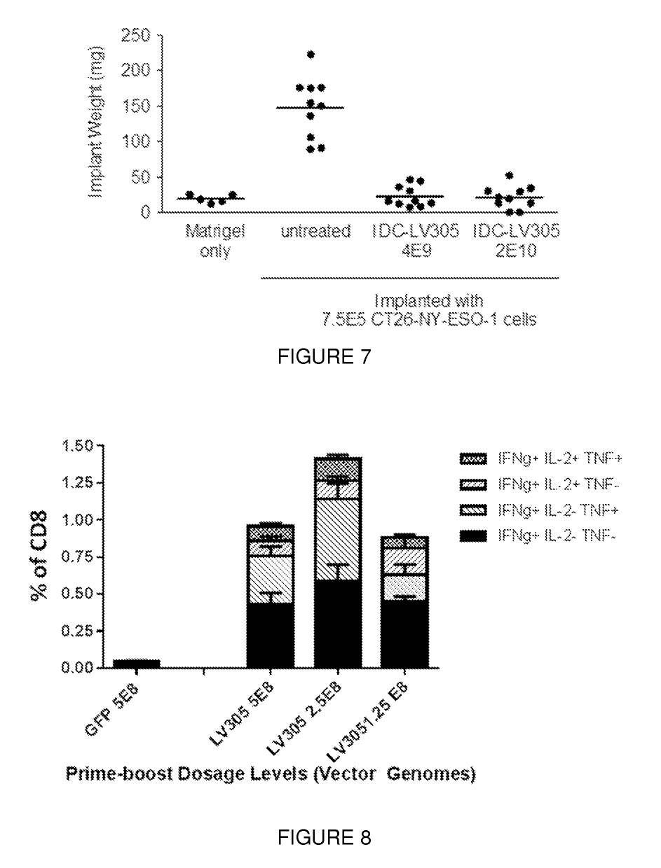

The minimally required dose required for prophylactically relevant anti-tumor responses in the murine tumor challenge model is unknown. BALB/c mice (n=10/group) were immunized with 4×10⁹ or 2×10¹⁰ vector genomes of ID-VP02-NY-ESO-1. At 21 days post immunization, immunized and untreated control mice were injected subcutaneously on the right flank with 7.5×10⁵ CT26-NY-ESO-1 tumor cells in Matrigel® or with Matrigel® alone as a negative control. Nine days after injection, the implants were excised and weighed to assess tumor growth. These results (FIG. 7) demonstrated that the lowest single dose of ID-VP02-NY-ESO-1 tested, 4×10⁹ genomes, was sufficient in eliciting significant rejection of NY-ESO-1 expressing tumor growth.

### Example 8

### Immunogenicity of ID-VP02-NY-ESO-1 in Mice Following a Single Subcutaneous Injection in Balb/C Mice

CD107a is a marker for cytotoxic T lymphocyte (CTL) activity in CD8+ T cells. BALB/c mice were immunized twice two weeks apart with ID-LV305 at the dosage levels 1.25×10⁸, 2.5×10⁸ and 5×10⁸ vector genomes. At 9 days after the second immunization, NY-ESO-1 splenic CD8 T cell responses were measured by surface staining of CD107a and intracellular cytokine staining (ICS) for IFN-γ, TNF-α, and IL-2 after ex vivo restimulation with the H-2D^{d}-restricted NY-ESO-1 epitope RGPESRLL (NY-ESO-1₈₁₋₈₈). Splenocytes from mice immunized with HBSS vehicle control and restimulated with NY-ESO-1₈₁₋₈₈ served as negative controls. As seen in FIG. 8, the lowest dose tested, 1.25×10⁸ vector genomes, was sufficient in eliciting antigen-specific T-cell responses following this prime-boost regimen.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim the following:

1. A method for inducing an immune response in a subject, the method comprising (a) administering to the subject at least two doses of a first immunogenic composition comprising (i) a lentiviral vector comprising a nucleotide sequence that encodes at least one immunogen or an immunogenic fragment thereof, wherein the at least one immunogen is capable of inducing an immune response specific for a first designated antigen; wherein the lentiviral vector is incorporated into a vector particle, and wherein the lentiviral vector particle is pseudotyped with an envelope glycoprotein that preferentially binds dendritic cells; (b) subsequently administering to the subject at least two doses of a second immunogenic composition comprising the at least one immunogen and a TLR4 agonist sequentially and at alternating times with two additional doses of the first immunogenic composition; thereby inducing an immune response specific for the first designated antigen.

2. The method of claim 1 wherein the at least two doses of the first immunogenic composition are each administered as a single injection.

3. The method of claim 1 wherein the at least two doses are administered intradermally or subcutaneously.

4. The method of claim one wherein the at least two doses of the first immunogenic composition are each administered as eight intradermal injections split between two injections over each deltoid and two over each quadricep.

5. The method of claim 1 where the at least two doses of the first immunogenic composition are administered 2 to 3 weeks apart and wherein at least one dose of the second immunogenic composition is administered 2 to 3 weeks following the second dose of the first immunogenic composition.

6. The method of claim 1 wherein the subject has cancer and the first designated antigen is a tumor antigen.

7. The method of claim 6 wherein the tumor antigen is selected from the group consisting of NY-ESO-1, MAGE-A3, MAGE-A1, MART-1/Melan-A, BAGE, RAGE, gp100, gp75, mda-7, tyrosinase, tyrosinase-related protein 2, renal cell carcinoma antigen, 5T4, SM22-alpha, carbonic anhydrase I, carbonic anhydrase IX, HIF-1alpha, HIF-2alpha, VEGF, prostate specific membrane antigen (PSMA), prostate-specific antigen (PSA), prostatic acid phosphates, EGFRvIII, WNT, Wilm's tumor antigen (WT1), six-transmembrane epithelial antigen of the prostate (STEAP), NKX3.1, telomerase enzyme, survivin, mesothelin, mutated ras, bcr/abl rearrangement, Her2/neu, mutated p53, wild-type p53, cytochrome P450 1B1, N-acetylglucosaminyltransferase-V, human papilloma virus protein E6, human papilloma virus protein E7, carcinoembryonic antigen, merkel cell virus T-antigen oncoproteins and alpha-fetoprotein.

8. The method of claim 1 wherein each of the at least two doses of the first immunogenic composition comprise from about 5×10⁸ to about 5×10¹⁰ vector genomes.

9. The method of claim 1 wherein each of the at least two doses of the first immunogenic composition comprise from about 5×10⁹ to about 1×10¹⁰ vector genomes.

10. The method of claim 1 wherein each of the at least two doses of the first immunogenic composition comprises about 1×10¹⁰ vector genomes.

11. The method of claim 1 wherein the lentiviral vector is integration deficient.

12. The method of claim 1 wherein the lentiviral vector is integration competent.

13. The method of claim 1 wherein the lentiviral vector is further characterized by any one or more or all of the following features: (a) pseudotyped with an envelope comprising a Sindbis virus E2 glycoprotein comprising an amino acid sequence having at least one amino acid change compared to SEQ ID NO:1, wherein residue 160 of SEQ ID NO:1 is either absent or an amino acid other than glutamic acid, and wherein the E2 glycoprotein is not a moiety of a fusion protein that comprises Sindbis virus E3 protein, (b) having a highly mannosylated envelope protein, optionally obtainable by culturing the packaging cells in a mannosidase inhibitor, (c) comprising a Vpx protein, optionally an SIVmac Vpx, (d) comprises a D64V integrase mutation within the gag/pol gene, (e) has a cPPT deletion within the vector genome, and (f) is optionally prepared using packaging cells comprising a rev-independent gag/pol system.

14. The method of claim 6 wherein the cancer is selected from the group consisting of renal cell carcinoma, prostate cancer, melanoma, and breast cancer.

15. The method of claim 1 wherein the TLR4 agonist is a compound of the following structure: 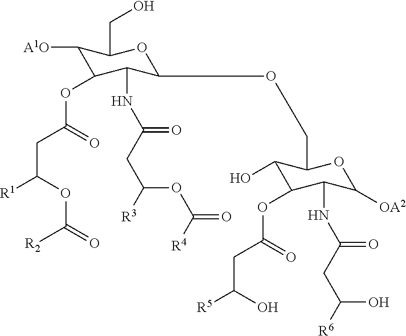
wherein A1 and A2 are independently selected from the group of hydrogen, phosphate, and phosphate salts and R1, R2, R3, R4, R5, and R6 are independently selected from the group of hydrocarbyl having 3 to 23 carbons, represented by C3-C23.

16. The method of claim 15, wherein A1 is phosphate or phosphate salt, A2 is hydrogen, R1, R3, R5 and R6 are undecyl and R2 and R4 are tridecyl.

17. The method of claim 16, wherein the compound is formulated in a stable oil-in-water emulsion.

18. The method of claim 17, wherein the second immunogenic composition comprises from about 5 ug GLA to about 10 ug GLA.

19. The method of claim 18, wherein each of the at least two doses of the first immunogenic composition comprises about 1×10¹⁰ vector genomes.

20. A method of treating a cancer in a mammal comprising administering at least a first dose of a composition comprising a lentiviral vector pseudotyped with an envelope glycoprotein that preferentially binds dendritic cells, wherein the lentiviral vector comprises an exogenous polynucleotide encoding a tumor antigen and wherein the at least a first dose is administered as a single injection; wherein the immune response specific for the tumor antigen elicited after administration of the first dose of the lentiviral vector as a single injection is greater than the immune response specific for the tumor antigen elicited after administration of the first dose split into multiple injections.


SEQUENCE LISTING (45 sequences)

SEQ ID NO: 1 - protein, 423 aa, Sindbis virus
  misc_feature (160)..(160): Xaa can be any naturally occurring amino acid
SVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKXTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 2 - protein, 986 aa, Sindbis virus
MSAAPLVTAMCLLGNVSFPCDRPPTCYTREPSRALDILEENVNHEAYDTLLNAILRCGSSGSVIDDFTLTSPYLGTCSYCHHTVPCFSPVKIEQVWDEADDNTIRIQTSAQFGYDQSGAASANKYRYMSLKQMYPYDVPDYATVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHAGYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 3 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVKEGTMDDIKISTSGPCRRLSYKGYFLLAKCPPGDSVTVSIVSSNSATSCTLARKIKPKFVGREKYDLPPVHGKKIPCTVYDRLEGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 4 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVEEGTMDDIKISTSGPCRRLSYKGYFLLAKCPPGDSVTVSIVSSNSATSCTLARKIKPKFVGREKYDLPPVHGKKIPCTVYDRLEGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 5 - protein, 980 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVKEGTMDDIKISTSGPCRRLSYKGYFLLAKCPPGDSVTVSIVSSNSATSCTLARKIKPKFVGREKYDLPPVHGKKIPCTVYDRLTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 6 - protein, 981 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVEEGTMDDIKISTSGPCRRLSYKGYFLLAKCPPGDSVTVSIVSSNSATSCTLARKIKPKFVGREKYDLPPVHGKKIPCTVYDRLETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 7 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLEGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 8 - protein, 981 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 9 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVEEGTMDDIKISTSGPCRRLSYKGYFLLAKCPPGDSVTVSIVSSNSATSCTLARKIKPKFVGREKYDLPPVHGKKIPCTVYDRLEGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 10 - protein, 981 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVEEGTMDDIKISTSGPCRRLSYKGYFLLAKCPPGDSVTVSIVSSNSATSCTLARKIKPKFVGREKYDLPPVHGKKIPCTVYDRLETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 11 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVEEGTMDDIKISTSGPCRRLSYKGYFLLAKCPPGDSVTVSIVSSNSATSCTLARKIKPKFVGREKYDLPPVHGKKIPCTVYDRLKGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 12 - protein, 981 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVEEGTMDDIKISTSGPCRRLSYKGYFLLAKCPPGDSVTVSIVSSNSATSCTLARKIKPKFVGREKYDLPPVHGKKIPCTVYDRLKTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 13 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVKEGTMDDIKISTSGPCRRLSYKGYFLLAKCPPGDSVTVSIVSSNSATSCTLARKIKPKFVGREKYDLPPVHGKKIPCTVYDRLKGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 14 - protein, 981 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVKEGTMDDIKISTSGPCRRLSYKGYFLLAKCPPGDSVTVSIVSSNSATSCTLARKIKPKFVGREKYDLPPVHGKKIPCTVYDRLKTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 15 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVEEGTMDDIKISTSGPCRRLSYKGYFLLAKCPPGDSVTVSIVSSNSATSCTLARKIKPKFVGREKYDLPPVHGKKIPCTVYDRLKGTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 16 - protein, 981 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLEQDHTVEEGTMDDIKISTSGPCRRLSYKGYFLLAKCPPGDSVTVSIVSSNSATSCTLARKIKPKFVGREKYDLPPVHGKKIPCTVYDRLKTTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 17 - protein, 982 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 18-19 - skipped (2 entries)

SEQ ID NO: 20 - protein, 488 aa, Sindbis virus
MSAAPLVTAMCLLGNVSFPCDRPPTCYTREPSRALDILEENVNHEAYDTLLNAILRCGSSGRSKRSVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 21 - DNA, 683 bp, Human immunodeficiency virus type 1
cctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 22 - DNA, 416 bp, Human immunodeficiency virus type 1
cctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 23 - DNA, 401 bp, Human immunodeficiency virus type 1
cctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttactggaagggctaattcactcccaacgaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 24-25 - skipped (2 entries)

SEQ ID NO: 26 - protein, 5 aa, Sindbis virus
RSKRS

SEQ ID NO: 27 - protein, 4 aa, Sindbis virus
RSKR

SEQ ID NO: 28-34 - skipped (7 entries)

SEQ ID NO: 35 - protein, 8 aa, Artificial Sequence
  a tag peptide sequence
DYKDDDDK

SEQ ID NO: 36 - protein, 1374 aa, Human herpesvirus 2
MAAPARDPPGYRYAAAILPTGSILSTIEVASHRRLFDFFAAVRSDENSLYDVEFDALLGSYCNTLSLVRFLELGLSVACVCTKFPELAYMNEGRVQFEVHQPLIARDGPHPVEQPVHNYMTKVIDRRALNAAFSLATEAIALLTGEALDGTGISLHRQLRAIQQLARNVQAVLGAFERGTADQMLHVLLEKAPPLALLLPMQRYLDNGRLATRVARATLVAELKRSFCDTSFFLGKAGHRREAIEAWLVDLTTATQPSVAVPRLTHADTRGRPVDGVLVTTAAIKQRLLQSFLKVEDTEADVPVTYGEMVLNGANLVTALVMGKAVRSLDDVGRHLLDMQEEQLEANRETLDELESAPQTTRVRADLVAIGDRLVFLEALERRIYAATNVPYPLVGAMDLTFVLPLGLFNPAMERFAAHAGDLVPAPGHPEPRAFPPRQLFFWGKDHQVLRLSMENAVGTVCHPSLMNIDAAVGGVNHDPVEAANPYGAYVAAPAGPGADMQQRFLNAWRQRLAHGRVRWVAECQMTAEQFMQPDNANLALELHPAFDFFAGVADVELPGGEVPPAGPGAIQATWRVVNGNLPLALCPVAFRDARGLELGVGRHAMAPATIAAVRGAFEDRSYPAVFYLLQAAIHGNEHVFCALARLVTQCITSYWNNTRCAAFVNDYSLVSYIVTYLGGDLPEECMAVYRDLVAHVEALAQLVDDFTLPGPELGGQAQAELNHLMRDPALLPPLVWDCDGLMRHAALDRHRDCRIDAGGHEPVYAAACNVATADFNRNDGRLLHNTQARAADAADDRPHRPADWTVHHKIYYYVLVPAFSRGRCCTAGVRFDRVYATLQNMVVPEIAPGEECPSDPVTDPAHPLHPANLVANTVKRMFHNGRVVVDGPAMLTLQVLAHNMAERTTALLCSAAPDAGANTASTANMRIFDGALHAGVLLMAPQHLDHTIQNGEYFYVLPVHALFAGADHVANAPNFPPALRDLARDVPLVPPALGANYFSSIRQPVVQHARESAAGENALTYALMAGYFKMSPVALYHQLKTGLHPGFGFTVVRQDRFVTENVLFSERASEAYFLGQLQVARHETGGGVNFTLTQPRGNVDLGVGYTAVAATGTVRNPVTDMGNLPQNFYLGRGAPPLLDNAAAVYLRNAVVAGNRLGPAQPLPVFGCAQVPRRAGMDHGQDAVCEFIATPVATDINYFRRPCNPRGRAAGGVYAGDKEGDVIALMYDHGQSDPARPFAATANPWASQRFSYGDLLYNGAYHLNGASPVLSPCFKFFTAADITAKHRCLERLIVETGSAVSTATAASDVQFKRPPGCRELVEDPCGLFQEAYPITCASDPALLRSARDGEAHARETHFTQYLIYDASPLKGLSL

SEQ ID NO: 37 - protein, 15 aa, Human herpesvirus 2
NYFSSIRQPVVQHAR

SEQ ID NO: 38 - protein, 15 aa, Human herpesvirus 2
CEFIATPVATDINYF

SEQ ID NO: 39 - protein, 15 aa, Human herpesvirus 2
ENALTYALMAGYFKM

SEQ ID NO: 40 - protein, 15 aa, Human herpesvirus 2
HPGFGFTVVRQDRFV

SEQ ID NO: 41 - protein, 8 aa, Artificial Sequence
  a tag peptide sequence
DLYDDDDK

SEQ ID NO: 42 - protein, 45 aa, Artificial Sequence
  Synthetic polypeptide
GPKEPFQSYVDRFYKSLRAEQTDAAVKNWMTQTLLIQNANPDCKL

SEQ ID NO: 43 - protein, 11 aa, Artificial Sequence
  Synthetic peptide
AAVKNWMTQTL

SEQ ID NO: 44 - protein, 9 aa, Artificial Sequence
  Synthetic peptide
KSLYNTVCV

SEQ ID NO: 45 - protein, 13 aa, Artificial Sequence
  Synthetic peptide
DRFYKSLRAEQTD